(19) United States
(12) Patent Application Publication
(10) Pub. No.: US 2010/0121307 A1
(43) Pub. Date: May 13, 2010

(54) Microneedles, Microneedle Arrays, Methods for Making, and Transdermal and/or Intradermal Applications

(75) Inventors: Michael S. Lockard, Lake Elizabeth, CA (US); Vacit Arat, La Canada Flintridge, CA (US); Adam L. Cohen, Los Angeles, CA (US); Kirk G. Nielsen, Newport Beach, CA (US)

Correspondence Address: MICROFABRICA INC.;ATT: DENNIS R. SMALLEY, 7911 HASKELL AVENUE, VAN NUYS, CA, 91406, US

(73) Assignee: Microfabrica Inc.

(21) Appl. No.: 12/611,108

(22) Filed: Nov. 2, 2009

Related U.S. Application Data
(63) Continuation-in-part of application No. 12/197,969, filed on Aug. 25, 2008.
(60) Provisional application No. 61/110,483, filed on Oct. 31, 2008.
(60) Provisional application No. 61/141,653, filed on Dec. 30, 2008.
(60) Provisional application No. 61/142,017, filed on Dec. 31, 2008.
(60) Provisional application No. 61/078,750, filed on Jul. 7, 2008.
(60) Provisional application No. 61/046,072, filed on Apr. 18, 2008.
(60) Provisional application No. 61/046,000, filed on Apr. 18, 2008.
(60) Provisional application No. 60/968,026, filed on Aug. 24, 2007.

(51) Int. Cl. A61M 5/32

(52) U.S. Cl. 604/506; 604/117; 604/22

(57) ABSTRACT

Embodiments are directed to microneedle array devices for intradermal and/or transdermal interaction with the body of patient to provide therapeutic, diagnostic or preventative treatment wherein portions of the devices may be formed by multi-layer, multi-material electrochemical fabrication methods and wherein individual microneedles may include valve elements or other elements for controlling interaction (e.g. fluid flow). In some embodiments needles are retractable and extendable from a surface of the device. In some embodiments, interaction occurs automatically with movement across the skin of the patient while in other embodiments interaction is controlled by an operator (e.g. doctor, nurse, technician, or patient).

### RELATED APPLICATIONS

This application claims benefit of U.S. Patent Application Nos. 61/110,483 (MF Docket No. P-US234-A-MF) filed Oct. 31, 2008; 61/141,653 (P-US252-A-MF) filed Dec. 30, 2008; and 61/142,017 (P-US241-A-MF) filed Dec. 31, 2008; and this application is a continuation-in-part of U.S. patent application Ser. No. 12/197,969 P-US232-A-MF), filed Aug. 25, 2008 which in turn claims the benefit of U.S. Patent Application Nos. 61/078,750 (P-US192-D-MF) filed Jul. 7, 2008; 61/046,072 (P-US192-C-MF), filed Apr. 18, 2008; 61/046,000 (P-US192-B-MF), filed Apr. 18, 2008; and 60/968,026 (P-US192-A-MF) filed Aug. 24, 2007. Each of these applications is incorporated herein by reference as if set forth in full herein.

### FIELD OF THE INVENTION

Embodiments of the invention relate to improved transdermal and/or intradermal drug delivery methods and systems with some embodiments directed to broad area, shallow depth, multi-needle, transdermal and/or intradermal delivery systems. Some embodiments are more particularly directed to apparatus that use such needles that are fabricated from multi-layer, multi-material deposition methods where subsequent layers are formed on previously formed layers and wherein each layer comprises at least two materials with at least one of the materials being a structural material and at least another one of the materials being a sacrificial material wherein the sacrificial material is removed from a plurality of the multiple layers after formation of the layers and wherein the formation of each layer includes a level setting operation (e.g. a planarization operation) which sets the level of the at least one structural material and the at least one sacrificial material.

### BACKGROUND OF THE INVENTION

### Electrochemical Fabrication

An electrochemical fabrication technique for forming three-dimensional structures from a plurality of adhered layers is being commercially pursued by Microfabrica® Inc. (formerly MEMGen Corporation) of Van Nuys, Calif. under the name EFAB®.

Various electrochemical fabrication techniques were described in U.S. Pat. No. 6,027,630, issued on Feb. 22, 2000 to Adam Cohen. Some embodiments of this electrochemical fabrication technique allow the selective deposition of a material using a mask that includes a patterned conformable material on a support structure that is independent of the substrate onto which plating will occur. When desiring to perform an electrodeposition using the mask, the conformable portion of the mask is brought into contact with a substrate, but not adhered or bonded to the substrate, while in the presence of a plating solution such that the contact of the conformable portion of the mask to the substrate inhibits deposition at selected locations. For convenience, these masks might be generically called conformable contact masks; the masking technique may be generically called a conformable contact mask plating process. More specifically, in the terminology of Microfabrica Inc. such masks have come to be known as INSTANT MASKS™ and the process known as INSTANT MASKING™ or INSTANT MASK™ plating. Selective depositions using conformable contact mask plating may be used to form single selective deposits of material or may be used in a process to form multi-layer structures. The teachings of the '630 patent are hereby incorporated herein by reference as if set forth in full herein. Since the filing of the patent application that led to the above noted patent, various papers about conformable contact mask plating (i.e. INSTANT MASKING) and electrochemical fabrication have been published:
(1) A. Cohen, G. Zhang, F. Tseng, F. Mansfeld, U. Frodis and P. Will, “EFAB: Batch production of functional, fully-dense metal parts with micro-scale features”, Proc. 9th Solid Freeform Fabrication, The University of Texas at Austin, p 161, August 1998.
(2) A. Cohen, G. Zhang, F. Tseng, F. Mansfeld, U. Frodis and P. Will, “EFAB: Rapid, Low-Cost Desktop Micromachining of High Aspect Ratio True 3-D MEMS”, Proc. 12th IEEE Micro Electro Mechanical Systems Workshop, IEEE, p 244, January 1999.
(3) A. Cohen, “3-D Micromachining by Electrochemical Fabrication”, Micromachine Devices, March 1999.
(4) G. Zhang, A. Cohen, U. Frodis, F. Tseng, F. Mansfeld, and P. Will, “EFAB: Rapid Desktop Manufacturing of True 3-D Microstructures”, Proc. 2nd International Conference on Integrated MicroNanotechnology for Space Applications, The Aerospace Co., April 1999.
(5) F. Tseng, U. Frodis, G. Zhang, A. Cohen, F. Mansfeld, and P. Will, “EFAB: High Aspect Ratio, Arbitrary 3-D Metal Microstructures using a Low-Cost Automated Batch Process”, 3rd International Workshop on High Aspect Ratio MicroStructure Technology (HARMST'99), June 1999.
(6) A. Cohen, U. Frodis, F. Tseng, G. Zhang, F. Mansfeld, and P. Will, “EFAB: Low-Cost, Automated Electrochemical Batch Fabrication of Arbitrary 3-D Microstructures”, Micromachining and Microfabrication Process Technology, SPIE 1999 Symposium on Micromachining and Microfabrication, September 1999.
(7) F. Tseng, G. Zhang, U. Frodis, A. Cohen, F. Mansfeld, and P. Will, “EFAB: High Aspect Ratio, Arbitrary 3-D Metal Microstructures using a Low-Cost Automated Batch Process”, MEMS Symposium, ASME 1999 International Mechanical Engineering Congress and Exposition, November, 1999.
(8) A. Cohen, “Electrochemical Fabrication (EFAB™)”, Chapter 19 of The MEMS Handbook, edited by Mohamed Gad-El-Hak, CRC Press, 2002.
(9) Microfabrication—Rapid Prototyping's Killer Application”, pages 1-5 of the Rapid Prototyping Report, CAD/CAM Publishing, Inc., June 1999.

The disclosures of these nine publications are hereby incorporated herein by reference as if set forth in full herein.

An electrochemical deposition for forming multilayer structures may be carried out in a number of different ways as set forth in the above patent and publications. In one form, this process involves the execution of three separate operations during the formation of each layer of the structure that is to be formed:
1. Selectively depositing at least one material by electrodeposition upon one or more desired regions of a substrate. Typically this material is either a structural material or a sacrificial material.
2. Then, blanket depositing at least one additional material by electrodeposition so that the additional deposit covers both the regions that were previously selectively deposited onto, and the regions of the substrate that did not receive any previously applied selective depositions. Typically this material is the other of a structural material or a sacrificial material.
3. Finally, planarizing the materials deposited during the first and second operations to produce a smoothed surface of a first layer of desired thickness having at least one region containing the at least one material and at least one region containing at least the one additional material.

After formation of the first layer, one or more additional layers may be formed adjacent to an immediately preceding layer and adhered to the smoothed surface of that preceding layer. These additional layers are formed by repeating the first through third operations one or more times wherein the formation of each subsequent layer treats the previously formed layers and the initial substrate as a new and thickening substrate.

Once the formation of all layers has been completed, at least a portion of at least one of the materials deposited is generally removed by an etching process to expose or release the three-dimensional structure that was intended to be formed. The removed material is a sacrificial material while the material that forms part of the desired structure is a structural material.

The preferred method of performing the selective electrodeposition involved in the first operation is by conformable contact mask plating. In this type of plating, one or more conformable contact (CC) masks are first formed. The CC masks include a support structure onto which a patterned conformable dielectric material is adhered or formed. The conformable material for each mask is shaped in accordance with a particular cross-section of material to be plated (the pattern of conformable material is complementary to the pattern of material to be deposited). At least one CC mask is used for each unique cross-sectional pattern that is to be plated.

The support for a CC mask is typically a plate-like structure formed of a metal that is to be selectively electroplated and from which material to be plated will be dissolved. In this typical approach, the support will act as an anode in an electroplating process. In an alternative approach, the support may instead be a porous or otherwise perforated material through which deposition material will pass during an electroplating operation on its way from a distal anode to a deposition surface. In either approach, it is possible for multiple CC masks to share a common support, i.e. the patterns of conformable dielectric material for plating multiple layers of material may be located in different areas of a single support structure. When a single support structure contains multiple plating patterns, the entire structure is referred to as the CC mask while the individual plating masks may be referred to as “submasks”. In the present application such a distinction will be made only when relevant to a specific point being made.

In preparation for performing the selective deposition of the first operation, the conformable portion of the CC mask is placed in registration with and pressed against a selected portion of (1) the substrate, (2) a previously formed layer, or (3) a previously deposited portion of a layer on which deposition is to occur. The pressing together of the CC mask and relevant substrate occur in such a way that all openings, in the conformable portions of the CC mask contain plating solution. The conformable material of the CC mask that contacts the substrate acts as a barrier to electrodeposition while the openings in the CC mask that are filled with electroplating solution act as pathways for transferring material from an anode (e.g. the CC mask support) to the non-contacted portions of the substrate (which act as a cathode during the plating operation) when an appropriate potential and/or current are supplied.

An example of a CC mask and CC mask plating are shown in FIGS. 1A-1C. FIG. 1A shows a side view of a CC mask 8 consisting of a conformable or deformable (e.g. elastomeric) insulator 10 patterned on an anode 12. The anode has two functions. One is as a supporting material for the patterned insulator 10 to maintain its integrity and alignment since the pattern may be topologically complex (e.g., involving isolated “islands” of insulator material). The other function is as an anode for the electroplating operation. FIG. 1A also depicts a substrate 6, separated from mask 8, onto which material will be deposited during the process of forming a layer. CC mask plating selectively deposits material 22 onto substrate 6 by simply pressing the insulator against the substrate then electrodepositing material through apertures 26a and 26b in the insulator as shown in FIG. 1B. After deposition, the CC mask is separated, preferably non-destructively, from the substrate 6 as shown in FIG. 10.

The CC mask plating process is distinct from a “through-mask” plating process in that in a through-mask plating process the separation of the masking material from the substrate would occur destructively. Furthermore in a through mask plating process, opening in the masking material are typically formed while the masking material is in contact with and adhered to the substrate. As with through-mask plating, CC mask plating deposits material selectively and simultaneously over the entire layer. The plated region may consist of one or more isolated plating regions where these isolated plating regions may belong to a single structure that is being formed or may belong to multiple structures that are being formed simultaneously. In CC mask plating as individual masks are not intentionally destroyed in the removal process, they may be usable in multiple plating operations.

Another example of a CC mask and CC mask plating is shown in FIGS. 1D-1G. FIG. 1D shows an anode 12′ separated from a mask 8′ that includes a patterned conformable material 10′ and a support structure 20. FIG. 1D also depicts substrate 6 separated from the mask 8′. FIG. 1E illustrates the mask 8′ being brought into contact with the substrate 6. FIG. 1F illustrates the deposit 22′ that results from conducting a current from the anode 12′ to the substrate 6. FIG. 1G illustrates the deposit 22′ on substrate 6 after separation from mask 8′. In this example, an appropriate electrolyte is located between the substrate 6 and the anode 12′ and a current of ions coming from one or both of the solution and the anode are conducted through the opening in the mask to the substrate where material is deposited. This type of mask may be referred to as an anodeless INSTANT MASK™ (AIM) or as an anodeless conformable contact (ACC) mask.

Unlike through-mask plating, CC mask plating allows CC masks to be formed completely separate from the substrate on which plating is to occur (e.g. separate from a three-dimensional (3D) structure that is being formed). CC masks may be formed in a variety of ways, for example, using a photolithographic process. All masks can be generated simultaneously, e.g. prior to structure fabrication rather than during it. This separation makes possible a simple, low-cost, automated, self-contained, and internally-clean “desktop factory” that can be installed almost anywhere to fabricate 3D structures, leaving any required clean room processes, such as photolithography to be performed by service bureaus or the like.

An example of the electrochemical fabrication process discussed above is illustrated in FIGS. 2A-2F. These figures show that the process involves deposition of a first material 2 which is a sacrificial material and a second material 4 which is a structural material. The CC mask 8, in this example, includes a patterned conformable material (e.g. an elastomeric dielectric material) 10 and a support 12 which is made from deposition material 2. The conformal portion of the CC mask is pressed against substrate 6 with a plating solution 14 located within the openings 16 in the conformable material 10. An electric current, from power supply 18, is then passed through the plating solution 14 via (a) support 12 which doubles as an anode and (b) substrate 6 which doubles as a cathode. FIG. 2A illustrates that the passing of current causes material 2 within the plating solution and material 2 from the anode 12 to be selectively transferred to and plated on the substrate 6. After electroplating the first deposition material 2 onto the substrate 6 using CC mask 8, the CC mask 8 is removed as shown in FIG. 2B. FIG. 2C depicts the second deposition material 4 as having been blanket-deposited (i.e. non-selectively deposited) over the previously deposited first deposition material 2 as well as over the other portions of the substrate 6. The blanket deposition occurs by electroplating from an anode (not shown), composed of the second material, through an appropriate plating solution (not shown), and to the cathode/substrate 6. The entire two-material layer is then planarized to achieve precise thickness and flatness as shown in FIG. 2D. After repetition of this process for all layers, the multi-layer structure 20 formed of the second material 4 (i.e. structural material) is embedded in first material 2 (i.e. sacrificial material) as shown in FIG. 2E. The embedded structure is etched to yield the desired device, i.e. structure 20, as shown in FIG. 2F.

Various components of an exemplary manual electrochemical fabrication system 32 are shown in FIGS. 3A-3C. The system 32 consists of several subsystems 34, 36, 38, and 40. The substrate holding subsystem 34 is depicted in the upper portions of each of FIGS. 3A-3C and includes several components: (1) a carrier 48, (2) a metal substrate 6 onto which the layers are deposited, and (3) a linear slide 42 capable of moving the substrate 6 up and down relative to the carrier 48 in response to drive force from actuator 44. Subsystem 34 also includes an indicator 46 for measuring differences in vertical position of the substrate which may be used in setting or determining layer thicknesses and/or deposition thicknesses. The subsystem 34 further includes feet 68 for carrier 48 which can be precisely mounted on subsystem 36.

The CC mask subsystem 36 shown in the lower portion of FIG. 3A includes several components: (1) a CC mask 8 that is actually made up of a number of CC masks (i.e. submasks) that share a common support/anode 12, (2) precision X-stage 54, (3) precision Y-stage 56, (4) frame 72 on which the feet 68 of subsystem 34 can mount, and (5) a tank 58 for containing the electrolyte 16. Subsystems 34 and 36 also include appropriate electrical connections (not shown) for connecting to an appropriate power source (not shown) for driving the CC masking process.

The blanket deposition subsystem 38 is shown in the lower portion of FIG. 3B and includes several components: (1) an anode 62, (2) an electrolyte tank 64 for holding plating solution 66, and (3) frame 74 on which feet 68 of subsystem 34 may sit. Subsystem 38 also includes appropriate electrical connections (not shown) for connecting the anode to an appropriate power supply (not shown) for driving the blanket deposition process.

The planarization subsystem 40 is shown in the lower portion of FIG. 3C and includes a lapping plate 52 and associated motion and control systems (not shown) for planarizing the depositions.

In addition to teaching the use of CC masks for electrodeposition purposes, the '630 patent also teaches that the CC masks may be placed against a substrate with the polarity of the voltage reversed and material may thereby be selectively removed from the substrate. It indicates that such removal processes can be used to selectively etch, engrave, and polish a substrate, e.g., a plaque.

The '630 patent further indicates that the electroplating methods and articles disclosed therein allow fabrication of devices from thin layers of materials such as, e.g., metals, polymers, ceramics, and semiconductor materials. It further indicates that although the electroplating embodiments described therein have been described with respect to the use of two metals, a variety of materials, e.g., polymers, ceramics and semiconductor materials, and any number of metals can be deposited either by the electroplating methods therein, or in separate processes that occur throughout the electroplating method. It indicates that a thin plating base can be deposited, e.g., by sputtering, over a deposit that is insufficiently conductive (e.g., an insulating layer) so as to enable subsequent electroplating. It also indicates that multiple support materials (i.e. sacrificial materials) can be included in the electroplated element allowing selective removal of the support materials.

The '630 patent additionally teaches that the electroplating methods disclosed therein can be used to manufacture elements having complex microstructure and close tolerances between parts. An example is given with the aid of FIGS. 14A-14E of that patent. In the example, elements having parts that fit with close tolerances, e.g., having gaps between about 1-5 um, including electroplating the parts of the device in an unassembled, preferably pre-aligned, state and once fabricated. In such embodiments, the individual parts can be moved into operational relation with each other or they can simply fall together. Once together the separate parts may be retained by clips or the like.

Another method for forming microstructures from electroplated metals (i.e. using electrochemical fabrication techniques) is taught in U.S. Pat. No. 5,190,637 to Henry Guckel, entitled “Formation of Microstructures by Multiple Level Deep X-ray Lithography with Sacrificial Metal layers”. This patent teaches the formation of metal structure utilizing through mask exposures. A first layer of a primary metal is electroplated onto an exposed plating base to fill a void in a photoresist (the photoresist forming a through mask having a desired pattern of openings), the photoresist is then removed and a secondary metal is electroplated over the first layer and over the plating base. The exposed surface of the secondary metal is then machined down to a height which exposes the first metal to produce a flat uniform surface extending across both the primary and secondary metals. Formation of a second layer may then begin by applying a photoresist over the first layer and patterning it (i.e. to form a second through mask) and then repeating the process that was used to produce the first layer to produce a second layer of desired configuration. The process is repeated until the entire structure is formed and the secondary metal is removed by etching. The photoresist is formed over the plating base or previous layer by casting and patterning of the photoresist (i.e. voids formed in the photoresist) are formed by exposure of the photoresist through a patterned mask via X-rays or UV radiation and development of the exposed or unexposed areas.

The '637 patent teaches the locating of a plating base onto a substrate in preparation for electroplating materials onto the substrate. The plating base is indicated as typically involving the use of a sputtered film of an adhesive metal, such as chromium or titanium, and then a sputtered film of the metal that is to be plated. It is also taught that the plating base may be applied over an initial layer of sacrificial material (i.e. a layer or coating of a single material) on the substrate so that the structure and substrate may be detached if desired. In such cases after formation of the structure the sacrificial material forming part of each layer of the structure may be removed along the initial sacrificial layer to free the structure. Substrate materials mentioned in the '637 patent include silicon, glass, metals, and silicon with protected semiconductor devices. A specific example of a plating base includes about 150 angstroms of titanium and about 300 angstroms of nickel, both of which are sputtered at a temperature of 160° C. In another example it is indicated that the plating base may consist of 150 angstroms of titanium and 150 angstroms of nickel where both are applied by sputtering.

Electrochemical Fabrication provides the ability to form prototypes and commercial quantities of miniature objects, parts, structures, devices, and the like at reasonable costs and in reasonable times. In fact, Electrochemical Fabrication is an enabler for the formation of many structures that were hitherto impossible to produce. Electrochemical Fabrication opens the spectrum for new designs and products in many industrial fields. Even though Electrochemical Fabrication offers this new capability and it is understood that Electrochemical Fabrication techniques can be combined with designs and structures known within various fields to produce new structures, certain uses for Electrochemical Fabrication provide designs, structures, capabilities and/or features not known or obvious in view of the state of the art.

A need exists in various fields for miniature devices having improved characteristics, reduced fabrication times, reduced fabrication costs, simplified fabrication processes, greater versatility in device design, improved selection of materials, improved material properties, more cost effective and less risky production of such devices, and/or more independence between geometric configuration and the selected fabrication process.

Micro-Needles:

Micro-needles and micro-needle arrays have been the subject of considerable development over the last decade or more. Micro-needles have been produced in both solid and hollow configurations. The latter offer several advantages such as no need for surface coating and continuous, longer-term delivery of material from an external reservoir. Hollow micro-needle arrays for drug delivery preferably have many desirable characteristics, such as for example:
Formable using a mature, commercial, highly-repeatable fabrication process;
Formable at reasonable cost in high volume production;
Formed of a material that is strong (so as not to shatter, bend, or buckle), non-brittle, biocompatible, and non-interacting with medications;
Possess adequate sharpness for easy tissue penetration;
Provide low hydraulic resistance in a reasonably-sized array;
Possess robustness,
Preferably possess a non-coring geometry (to minimize tissue damage and avoid plugging of outlet holes);
Have appropriate length and penetration control for a given task;
Preferably have reasonably smooth internal surfaces; and
Be capable of sterilization by cost-effective methods.

In recent years, a number of researchers have attempted to develop hollow micro-needles and micro-needle arrays that could meet these requirements, but there have been many challenges. For example, many efforts have centered on producing micro-needles using silicon. However, it has been found that silicon's intrinsically high brittleness presents an absolute barrier to producing micro-needles that are safe (i.e. cannot shatter) inside the skin, leaving behind shards which can create irritation or infection, and making accurate dosing a challenge. Silicon's brittleness alone virtually disqualifies it as a viable material for micro-needles. Moreover, fabricating non-coring, pre-assembled/ready-to-use micro-needle arrays with low flow resistance requires fabricating relatively complex 3-D geometries: a difficult task using silicon. The Nanopass (Nes-Ziona, Israel) “Micropyramid” needle seems likely to core tissue or at least be subject to plugging. Meanwhile, the Debiotech (Lausanne, Switzerland) silicon “Nanoject” needle does use a side port, but its fabrication process involves many costly steps). Finally, silicon is a costly material with costly processing (e.g., deep reactive ion etching): well suited to making high-value computer chips but not so well suited to creating affordable alternatives to commodity products such as hypodermic needles.

Other efforts have focused on making hollow micro-needles from polymers or glass. In the case of polymers, strength, sharpness, geometrical limitations, and difficulties in sterilization have been issues, and in the case of glass, brittleness and geometrical limitations have prevented serious adoption.

Because of the limitations of silicon, polymers, and glass, several more recent efforts to produce hollow micro-needle arrays have centered on using metals. Metals, of course, are well established for fabrication of hypodermic needles and have a long safety record in such use. Metals are generally ductile, eliminating risk of brittle fracture—a major barrier with use of silicon and glass. Metals are also relatively inexpensive, have very high strength, may be highly sharpened, can be fabricated into complex shapes, and are readily capable of being sterilized by known methods.

The most common approach to fabricating hollow metal micro-needles is by electroplating into or onto molds. For example, Georgia Institute of Technology has done considerable work in the area of plating metal into molds made of silicon or polymer. However, the devices produced have been wanting, and the processes remain laboratory-scale and are not commercialized. Most of Georgia Tech's devices have been fabricated by plating thin metal into a mold. The simple geometries available have made the use of side ports for drug release impossible to achieve; thus all such devices release drug through a single port at the needle tip and are subject to tissue coring and plugging. Moreover, the use of molds for these needles introduces some problems. Silicon molds are costly to produce, a problem particularly if they are for a single use, while polymer molds (especially produced using laser machining) typically have rough, non-repeatable geometries and poor surface finishes. Laboratory efforts at the University of Texas, Dallas have produced metallic micro-needle arrays by plating metal onto thick photoresist, with the photoresist ultimately removed by a prolonged plasma etch subsequent to a planarization operation to expose the tips. However, the result is a needle of questionable sharpness (wall thickness is 10-20 μm) and with a single port at the tip which is subject to coring and plugging.

Alternative efforts (at Georgia Tech) to produce a metal needle with side ports have been somewhat successful, but are limited to making individual needles or 1-D (vs. 2-D) arrays. Moreover, the small lumens produce substantial hydraulic resistance. In the commercial realm, Becton, Dickinson and Company (Franklin Lakes, N.J.) appears to have a program to develop hollow metal micro-needles; these seem to be essentially miniaturized hypodermic needles, and an economical process for building arrays of these may be problematic. Also, the needles may produce tissue coring. Metals used for plated metal devices reported to date either have been of unacceptably low biocompatibility (e.g., pure nickel) or are potentially costly (e.g., palladium).

### SUMMARY OF THE INVENTION

It is an object of some embodiments of the invention to provide an improved method for providing shallow intradermal and/or transdermal (i.e. under 2 mm and preferably under 400 microns) injections of desired materials or drugs or extraction of fluids controllably from an array of micro-needles. These improved methods may involve, for example, the use of one or more of” (1) non-coring needles, (2) needles having diameters and tips that are small enough to substantially reduce or even eliminate pain associated with insertion, (3) needles that have a controllable and reliable insertion depth, (4) needle arrays that limit dispensing to that portion of the needles that have properly entered the target surface; (5) needle arrays that minimize drug delivery from needles that have not properly engaged the target surface; or (6) meet one of the other desirable features noted above in the background section of this application or meet another beneficial criteria that will be apparent to one of skill in the art upon review of the teachings herein.

It is an object of some embodiments of the invention to provide an improved devices capable of providing shallow intradermal and/or transdermal (i.e. under 2 mm and preferably under 400 microns) injections of desired materials or drugs or extraction of fluids controllably from an array of micro-needles. These devices, for example, may include one or more of the features noted above with regard to the method objectives of some embodiments as set forth above.

It is an object of some embodiments of the invention to provide improved methods of applying micro-needles or micro-needle arrays to specific locations on the skin of a patient and to use such needles to provide therapeutic or preventive treatment to the patient in, for example, one or more of the applications areas of: (1) delivery of antibiotics, (2) delivery of antipruritic agents, (3) delivery of anti-inflammatory agents, (4) delivery of analgesics, (5) treatment of abscesses, (6) removal of lesions such as moles, (7) relieve edema, (8) delivery of depilatory agents to the roots of unwanted hairs, (9) removal of tattoos via dye delivery, and/or (10) direct delivery of hair re-growth agents. Other applications areas may include diagnostics, such as, for example: (1) allergy testing, (2) electric signal detection, e.g. from muscles; and/or (3) fluid extraction for current or subsequent testing.

Other objects and advantages of various embodiments of the invention will be apparent to those of skill in the art upon review of the teachings herein. The various embodiments of the invention, set forth explicitly herein or otherwise ascertained from the teachings herein, may address one or more of the above objects alone or in combination, or alternatively may address some other object ascertained from the teachings herein. It is not necessarily intended that all objects be addressed by any single aspect of the invention even though that may be the case with regard to some aspects.

A first aspect of the invention provides a device for the intradermal and/or transdermal dispensing of a drug into a body of a patient through a desired delivery area, including: (a) a handle; (b) a cylindrical body having one or more apertures extending from an interior region to an exterior region wherein the cylindrical body and the handle are joined to one another such that relative rotation between the handle and body may occur; (c) a plurality of needles extending outward from the one or more apertures, wherein the needles and cylindrical body are configured to provide a desired penetration depth into a surface of a delivery area when the cylindrical body is rolled over the delivery area as the handle is translated relative to the delivery area; wherein the device further includes at least one element taken from the group of elements consisting of: (1) at least a portion of some of the needles are formed from a multi-layer, multi-material fabrication process where each of the multiple layers are formed from the deposition of at least one structural material and at least one sacrificial material, a trimming (e.g. planarization) of the at least one structural material and the at least one sacrificial material to set a boundary level for the layer, and wherein after formation of a plurality of layers, the sacrificial material is removed from the plurality of layers; (2) the needles include penetration stops located proximally relative to more distal apertures in the needles such that the distance between the apertures and the penetration stops defines a desired delivery depth for the drug below surface of the delivery area; (3) the outer surface of the cylindrical body is covered with a membrane that inhibits flow of the drug from the needles except in those locations where the needles have been made to extend through the membrane by contact with the delivery area; (4) the outer surface of the cylindrical body is covered with a compressible membrane that inhibits flow of the drug from the needles except in those locations where the needles have been made to extend through the membrane by compression of the membrane against the delivery area; (5) the distal ends of the needles are made to extend from an interior position relative to the apertures in the cylindrical body via one or more bulges in one or more tracks along which with the needles move as the cylindrical body rotates with respect to the handle wherein the bulge or bulges correspond to a location along the perimeter of the cylindrical body that is intended to be in contact with a delivery area during a drug delivery; (6) a drug is held within the cylindrical body within pores of a flexible porous medium and is forced from selected needles by squeezing the porous medium adjacent the selected needles; (7) selected needles are extended from selected apertures via fluid pressure exerted by the drug on bases of the selected needles; (8) a non-rotating inter conformable structure is held with the cylindrical structure wherein the conformable structure includes a recess which acts as a reservoir for the drug and which is configured and located so that it is positioned adjacent to only a portion of the needles and in particular that portion of the needles that are located in a desired dispensing position and wherein other portions of the conformable structure act to inhibit other needles from dispensing the drug; (9) the distal ends of the needles are made to extend from an interior position relative to the apertures in the cylindrical body such that they extend beyond the outer surface of the cylindrical body for penetration into the tissue through the delivery area when the cylindrical body is in contact with the delivery area; (10) a plurality of normally closed valves (i.e. when no dispensing is to occur) which can be made to open to allow fluid flow through selected needles when those needles have penetrated the tissue at the delivery area; (11) a plurality of normally closed valves (i.e. when no dispensing is to occur) which can be made to open to allow fluid flow through selected needles when those needles have penetrated the tissue at the delivery area, wherein selected valves are made to open by a plurality of trigger mechanisms being depressed as the cylindrical body is pressed against the delivery area; (12) a plurality of normally closed valves (i.e. when no dispensing is to occur) which can be made to open to allow fluid flow through selected needles when those needles have penetrated the tissue at the delivery area, wherein selected valves include valve seats that seal the exit ports of the needles when trigger mechanisms are not sufficiently depressed; (13) a plurality of normally closed valves (i.e. when no dispensing is to occur) which can be made to open to allow fluid flow through selected needles when those needles have penetrated the tissue at the delivery area, wherein selected valves include valve seats that seal the fluid entry ports of the needles when trigger mechanisms are not sufficiently depressed; and (14) the plurality of needles are held within the cylindrical body and are made to extend from the cylindrical body in a region of the cylindrical body that is in contact with the delivery area prior to dispensing the drug and after dispensing the drug are made to retract back into the cylindrical body.

A second aspect of the invention provides a device for the intradermal and/or transdermal dispensing of a drug over desired delivery area, including: (a) a handle; (b) a smooth body having a plurality of apertures extending from an interior region to an exterior region wherein the smooth body and the handle are joined to one another such that relative rotation between the handle and body may occur; (c) a plurality of needles extending outward from the plurality of apertures; wherein the needles and smooth body are configured to provide a desired penetration depth and into a surface of the delivery area when the smooth body is rolled over the delivery area as the handle is translated relative to the delivery area; wherein the device further includes at least one element taken from the group of elements consisting of: (1) at least a portion of some of the needles are formed from a multi-layer, multi-material fabrication process where each of the multiple layers is formed from the deposition of at least one structural material and at least one sacrificial material, a trimming (e.g. planarization) of the at least one structural material and the at least one sacrificial material sets a boundary level for the layer, and wherein after formation of a plurality of layers the sacrificial material is removed from the plurality of layers; (2) the needles include penetration stops that are located proximally from more distal apertures such that the distance between the apertures and the penetration stops defines a desired delivery depth within the tissue for the drug; (3) the outer surface of the smooth body is covered with a membrane that inhibits flow of the drug from the needles except in those locations where the needles have been made to extend through the membrane by contact with the delivery area; (4) the outer surface of the smooth body is covered with a compressible membrane that inhibits flow of the drug from the needles except in those locations where the needles have been made to extend through the membrane by compression of the membrane against the delivery area; (5) the distal ends of the needles are made to extend from an interior position relative to the apertures in the smooth body via a bulge in one or more tracks along which with the needles move as the smooth body rotates with respect to handle wherein the bulge or bulges correspond to a location along the perimeter of the smooth body that is intended to be in contact with a delivery area during a drug delivery; (6) a drug is held within the smooth body within pores of a flexible porous medium and is forced from selected needles by squeezing the porous medium adjacent the selected needles; (7) selected needles are extended from selected apertures via fluid pressure exerted by the drug on bases of the selected needles; (8) a non-rotating conformable structure is held within the smooth structure wherein the conformable structure includes a recess which acts as a reservoir for the drug and which is configured and located so that it is positioned adjacent to only a portion of the needles and in particular that portion of the needles that are located in a desired dispensing position and wherein other portions of the conformable structure act to inhibit other needles from dispensing the drug; (9) the distal ends of the needles are made to extend from an interior position relative to the apertures in the smooth body such that they extend beyond the outer surface of the smooth body for penetration into the tissue through the delivery area when the smooth body is in contact with the delivery area; (10) a plurality of normally closed valves (i.e. when no dispensing is to occur) which can be made to open to allow fluid flow through selected needles when those needles have penetrated the tissue at the delivery area; (11) a plurality of normally closed valves (i.e. when no dispensing is to occur) which can be made to open to allow fluid flow through selected needles when those needles have penetrated the tissue at the delivery area, wherein selected valves are made to open by a plurality of trigger mechanisms being depressed as the smooth body is pressed against the delivery area; (12) a plurality of normally closed valves (i.e. when no dispensing is to occur) which can be made to open to allow fluid flow through selected needles when those needles have penetrated the tissue at the delivery area, wherein selected valves include valve seats that seal the exit ports of the needles when trigger mechanisms are not sufficiently depressed; (13) a plurality of normally closed valves (i.e. when no dispensing is to occur) which can be made to open to allow fluid flow through selected needles when those needles have penetrated the tissue at the delivery area, wherein selected valves include valve seats that seal the fluid entry ports of the needles when trigger mechanisms are not sufficiently depressed; and (14) the plurality of needles are held within the cylindrical body and are made to extend from the cylindrical body in a region of the smooth body that is in contact with the delivery area prior to dispensing the drug and after dispensing the drug are made to retract back into the smooth body.

A third aspect of the invention provides a device for the intradermal and/or interdermal dispensing of a drug over desired delivery area, including: (a) a handle; (b) a smooth body having one or more apertures extending from an interior region to an exterior region wherein the smooth body and the handle are joined to one another such that the smoothed body extends beyond a surface of a body of the handle, (c) a plurality of needles which are extendible from the smooth body and retractable into the smooth body via the one or more apertures such that upon extension the drug may be dispensed and such that upon retraction the smooth body may be translated along the surface of the delivery area, wherein the needles and smooth body are configured to provide a desired penetration depth of the needles into a surface of the delivery area when the needles are extended; wherein the device further includes an element taken from the group of elements consisting of: (1) at least a portion of some of the needles are formed from a multi-layer, multi-material fabrication process where each of the multiple layers is formed from the deposition of at least one structural material and at least one sacrificial material, a trimming process removes a portion of the at least one structural material and the at least one sacrificial material to set a boundary level for the layer, and wherein after formation of a plurality of layers the sacrificial material is removed from the plurality of layers; (2) the needles include penetration stops located proximally from more distal apertures such that the distance between the apertures and the penetration stops defines a desired delivery depth for the drug; (3) the outer surface of the smooth body is covered with a membrane that inhibits flow of the drug from the needles except in those locations where the needles have been made to extend through the membrane by contact with the delivery area; (4) the outer surface of the smooth body is covered with a compressible membrane that inhibits flow of the drug from the needles except in those locations where the needles have been made to extend through the membrane by compression of the membrane against the delivery area; (5) a drug is held within the smooth body within pores of a flexible porous medium and is forced from selected needles by squeezing the porous medium adjacent the selected needles; (6) selected needles are extended from selected apertures via fluid pressure exerted by the drug on bases of the selected needles; (7) the distal ends of the needles are made to extend from an interior position relative to the apertures in the smooth body such that they extend beyond the outer surface of the smooth body for penetration into the tissue through the delivery area when the smooth body is in contact with the delivery area; (8) a plurality of normally closed valves (i.e. when no dispensing is to occur) which can be made to open to allow fluid flow through selected needles when those needles have penetrated the tissue at the delivery area; (9) a plurality of normally closed valves (i.e. when no dispensing is to occur) which can be made to open to allow fluid flow through selected needles when those needles have penetrated the tissue at the delivery area, wherein selected valves are made to open by a plurality of trigger mechanisms being depressed as the smooth body is pressed against the delivery area; (10) a plurality of normally closed valves (i.e. when no dispensing is to occur) which can be made to open to allow fluid flow through selected needles when those needles have penetrated the tissue at the delivery area, wherein selected valves include valve seats that seal the exit ports of the needles when trigger mechanisms are not sufficiently depressed; (12) a plurality of normally closed valves (i.e. when no dispensing is to occur) which can be made to open to allow fluid flow through selected needles when those needles have penetrated the tissue at the delivery area, wherein selected valves include valve seats that seal the fluid entry ports of the needles when trigger mechanisms are not sufficiently depressed; and (13) the plurality of needles are held within the cylindrical body and are made to extend from the cylindrical body in a region of the smooth body that is in contact with the delivery area prior to dispensing the drug and after dispensing the drug are made to retract back into the smooth body.

A fourth aspect of the invention provides a device for the intradermal and/or interdermal interaction with a body of a patient, including: (a) a handle; (b) a smooth body having one or more apertures extending from an interior region to an exterior region wherein the smooth body and the handle are functionally connected to one another such that the smoothed body extends beyond a surface of a body of the handle; (c) a plurality of needles which are extendible from the smooth body and retractable into the smooth body via the one or more apertures such that upon extension a desired intradermal or interdermal interaction can occur via passages within the needles and such that upon retraction the smooth body may be translated or rotated along the surface of the delivery area, wherein the needles and smooth body are configured to provide a desired penetration depth of the needles into a surface of the delivery area when the needles are extended; wherein the device further includes an element taken from the group of elements consisting of: (1) at least a portion of some of the needles are formed from a multi-layer, multi-material fabrication process where each of the multiple layers is formed from the deposition of at least one structural material and at least one sacrificial material, a trimming (e.g. planarization) of the at least one structural material and the at least one sacrificial material sets a boundary level for the layer, and wherein after formation of a plurality of layers the sacrificial material is removed from the plurality of layers; (2) the needles include penetration stops that are located proximally from more distal apertures such that the distance between the apertures and the penetration stops defines a desired interaction depth within the tissue; (3) the outer surface of the smooth body is covered with a membrane that inhibits interaction between the tissue and the needles except in those locations where the needles have been made to extend through the membrane by contact with the delivery area; (4) the outer surface of the smooth body is covered with a compressible membrane that inhibits interaction between the needles and the tissue except in those locations where the needles have been made to extend through the membrane by compression of the membrane against the delivery area; (5) the distal ends of the needles are made to extend from an interior position relative to the apertures in the smooth body via a bulge in one or more tracks along which with the needles move as the smooth body rotates with respect to handle wherein the bulge or bulges correspond to a location along the perimeter of the smooth body that are intended to be in contact with a delivery area an interaction; (6) a drug is held within the smooth body within pores of a flexible porous medium and is forced from selected needles during an interaction by squeezing the porous medium adjacent the selected needles; (7) selected needles are extended from selected apertures via fluid pressure exerted by a drug on bases of the selected needles wherein the drug is to be dispensed during an interaction; (8) a non-rotating conformable structure is held within the smooth structure wherein the conformable structure includes a recess which acts as a reservoir for a drug to be dispensed during an interaction and which is configured and located so that the conformable structure is positioned adjacent to only a portion of the needles and in particular that portion of the needles that are located in a desired dispensing position and wherein other portions of the conformable structure act to inhibit other needles from dispensing the drug; (9) the distal ends of the needles are made to extend from an interior position relative to the apertures in the smooth body such that they extend beyond the outer surface of the smooth body for penetration into the tissue through the delivery area when the smooth body is in contact with the delivery area; (10) a plurality of normally closed valves which can be made to open to allow fluid flow through selected needles when those needles have penetrated the tissue at the delivery area, wherein the direction of fluid flow is selected from the group consisting of outward flow and an inward flow; (11) a plurality of normally closed valves which can be made to open to allow fluid flow through selected needles when those needles have penetrated the tissue at the delivery area, wherein selected valves are made to open by a plurality of trigger mechanisms being depressed as the smooth body is pressed against the delivery area, wherein the direction of fluid flow is selected from the group consisting of outward flow and an inward flow; (12) a plurality of normally closed valves which can be made to open to allow fluid flow through selected needles when those needles have penetrated the tissue at the delivery area, wherein selected valves include valve seats that seal the exit ports of the needles when trigger mechanisms are not sufficiently depressed, wherein the direction of fluid flow is selected from the group consisting of outward flow and an inward flow; (13) a plurality of normally closed valves which can be made to open to allow fluid flow through selected needles when those needles have penetrated the tissue at the delivery area, wherein selected valves include valve seats that seal the fluid entry ports of the needles when trigger mechanisms are not sufficiently depressed, wherein the direction of fluid flow is selected from the group consisting of outward flow and an inward flow; (14) the plurality of needles are held within the cylindrical body and are made to extend from the cylindrical body in a region of the smooth body that is in contact with the delivery area prior to interaction between the needles and the tissue and after interaction are made to retract back into the smooth body; (15) the interaction includes directing radiation down one or more the plurality of needles; (16) the interaction includes reading an electrical signal from one or more of the needles; (17) the interaction includes applying a voltage to one or more of the needles; (18) the interaction includes a diagnostic procedure; (19) the interaction includes a preventative treatment; and (20) the interaction includes a therapeutic treatment.

A variation of the 1^{st} through 4^{th} aspects includes a device wherein the device includes at least two of the elements.

A variation of the 1^{st} through 4^{th} aspects includes a device wherein the device includes at least three of the elements.

A fifth aspect of the invention provides a method for supplying therapeutic or preventative treatment to a body of a patient, comprising: (a) applying the surface of a device to the skin of a patient in a desired working area; (b) extending a plurality of microneedles from the surface of the device through the skin to a desired interaction area; (c) providing a therapeutic treatment, a preventative treatment to the patent via an interaction wherein the interaction is selected from the group including: (1) delivering antibiotics; (2) delivering of antipruritic agent; (3) delivering of anti-inflammatory agent; (4) delivering of an analgesic; (5) treating an abscess; (6) removal of a lesion; (7) providing a treatment to relieve edema; (8) delivering depilatory agents to the roots of unwanted hairs; (9) removing tattoos via dye delivery; (10) delivering hair re-growth agents; (11) providing a treatment for hyperhydrosis; (12) delivery of insulin; (13) delivery of a vaccine; (14) delivery of a growth hormone; (15) delivery of an anti-osteoporosis drug; (16) delivery a chemotherapy agent; (17) delivery of a thrombolytic agent; (18) delivery of a blood pressure regulation drug; (19) delivery of an addiction therapy drug; (20) delivery of nanoparticles; (21) delivery of nanoparticles for use in a cancer treatment; (22) performing allergy testing; (23) delivery of radioactive drugs; (24) delivery of a drug with a narrow therapeutic range; and (12) providing an in vivo sensing of a body fluid for detection of the presence of a substance of interest.

A variation of the 5^{th} aspect includes a method wherein the device includes the device of any of the 1^{st}-4^{th} aspects and their variations.

The disclosure of the present invention provides a number of primary embodiments wherein needles and/or valve elements and other elements may be formable from a plurality of adhered layers where each successive layer comprising at least two materials, one of which is a structural material and the other of which is a sacrificial material, and wherein each successive layer defines a successive cross-section of the three-dimensional structure, and wherein the forming of each of the plurality of successive layers includes: (i) depositing a first of the at least two materials; (ii) depositing a second of the at least two materials; and (B) after the forming of the plurality of successive layers, separating at least a portion of the sacrificial material from the structural material to reveal the three-dimensional structure. In these embodiments, each layer may also include one or more trimming operations that are used to set the boundary of a layer by setting the height of at least one sacrificial material and at least one structural material to a common level.

Other aspects of the invention will be understood by those of skill in the art upon review of the teachings herein. Other aspects of the invention may involve combinations of the above noted aspects of the invention. These other aspects of the invention may provide various combinations of the aspects presented above as well as provide other configurations, structures, functional relationships, and processes that have not been specifically set forth above.

### BRIEF DESCRIPTION OF THE DRAWINGS

FIGS. 1A-1C schematically depict side views of various stages of a CC mask plating process, while FIGS. 1D-G schematically depict a side views of various stages of a CC mask plating process using a different type of CC mask.

FIGS. 2A-2F schematically depict side views of various stages of an electrochemical fabrication process as applied to the formation of a particular structure where a sacrificial material is selectively deposited while a structural material is blanket deposited.

FIGS. 3A-3C schematically depict side views of various example subassemblies that may be used in manually implementing the electrochemical fabrication method depicted in FIGS. 2A-2F.

FIGS. 4A-4F schematically depict the formation of a first layer of a structure using adhered mask plating where the blanket deposition of a second material overlays both the openings between deposition locations of a first material and the first material itself

FIG. 4G depicts the completion of formation of the first layer resulting from planarizing the deposited materials to a desired level.

FIGS. 4H and 4I respectively depict the state of the process after formation of the multiple layers of the structure and after release of the structure from the sacrificial material.

FIGS. 5A and 5B provide perspective views of a device according to some embodiments of the invention. The device includes a handle, a housing, a trigger button for extending needles and dispensing material, and a slide or cylindrical body on which one or more needle arrays are mounted or from which they are extendable and which are capable of dispensing or extracting a desired drug or fluid material.

FIG. 5C provides a perspective view of a single linear needle array extending from the slide. During use, the needles are retracted into the slide or cylindrical body during movement across the delivery site and are extended from the slide or cylindrical body during dispensing times.

FIGS. 6A and 6B provide perspective views of a device according to some embodiments of the invention (e.g. the third group or seventh group of embodiments). The device includes a handle, a housing, and a plurality of independently supported slides or rings (as shown) or cylindrical body on which one or more needle arrays are mounted and are capable of dispensing or extracting a desired drug or fluid material.

FIG. 6C provides a perspective view of a single linear needle array extending from the slides or rings of the group of embodiments of FIGS. 6A and 6B.

FIG. 6D depicts additional elements found in the embodiments of FIGS. 6A-6C which include independent leaf spring for each of the slides or rings to allow more intimate contact with a target surface along with the delivery site along the width of the needle array.

FIGS. 7A-7D provide two examples of flow restriction and needle extension and retraction options that may be used in conjunction with some device embodiments of the invention.

FIGS. 8A-8C provide various schematic views of a needle array according to an embodiment of the invention wherein individual needles include valve elements that inhibit flow of fluid from the needles if the needles are not pressed into or against a target surface.

FIGS. 9A-9C provide schematic illustrations of various states of a needle array configuration according to another embodiment of the invention where the needles include alternative valve elements which inhibit the flow of fluid from the needles when the needles are not pressed into or against a delivery surface.

FIGS. 10A-10B provide two additional illustrations of retractable needle embodiments.

FIGS. 11A-11C depict, respectively, a perspective view of a needle 1100, a cut view of the needle of showing internal passages, and an array formed from such needles for use in some embodiments of the invention.

FIGS. 12A-12D depict a needle 1200 and an array of needles such needles according to a second example of needles that may be used in some embodiments of the invention.

FIGS. 13A-13B provide schematic view of other needle tip designs according to first and second implementation of a third example sample of needle configurations that may help promote retention of the needle within tissue.

FIG. 13C provides a view of a needle according to a third implementation of the third set of examples of needles configurations that may be used in embodiments of the invention where some of the outlet passages have back facing openings (i.e. openings that are directed, at least in part, toward the proximal end of the needle).

FIG. 14A provides a perspective of a smooth walled CAD design of a sample needle design of an fourth example implementation of a needle configuration that may be used in some embodiments of the invention wherein the configuration provides some features in common with the example of FIGS. 11A-11C but where the configuration provides secondary edges that are also sharp.

FIG. 14B provides a perspective view of the needle device of FIG. 14A after cross-sectioning the needle into 17 layers.

FIGS. 15A-15B provide schematic illustrations of a sample array of needles which are formed in a laterally compressed state (FIG. 15A) which may be connected by flexures and separated to a desired array spacing after formation (FIG. 15B) as an example of a probe array configuration that may be used in some embodiments of the invention wherein higher lateral needle density is achieved during fabrication with associated cost, time, and/or material consumption savings.

FIGS. 16A-16B provides schematic views of two alternative techniques for holding a needle array base spaced from a surface of the tissue in which the needle arrays are inserted according to a first illustration of a sixth set of example implementations of needle configurations that may be used in some embodiments of the invention.

FIG. 17A provides a perspective view of a linear needle array according another illustration of the sixth set of example implementations of needle configurations that may be used in some embodiments of the invention.

FIG. 17B provides a perspective view of a circular needle array according another illustration of the sixth set of example implementations of needle configurations that may be used in some embodiments of the invention.

FIGS. 18A-18B provide a perspective views of needles according two illustrations of the seventh set of example implementations of needle configurations that may be used in some embodiments of the invention.

FIGS. 19A-19B provide schematic illustrations of needles according an illustration of the eighth set of example implementations of needle configurations that may be used in some embodiments of the invention.

### DETAILED DESCRIPTION OF PREFERRED EMBODIMENTS

### Electrochemical Fabrication in General

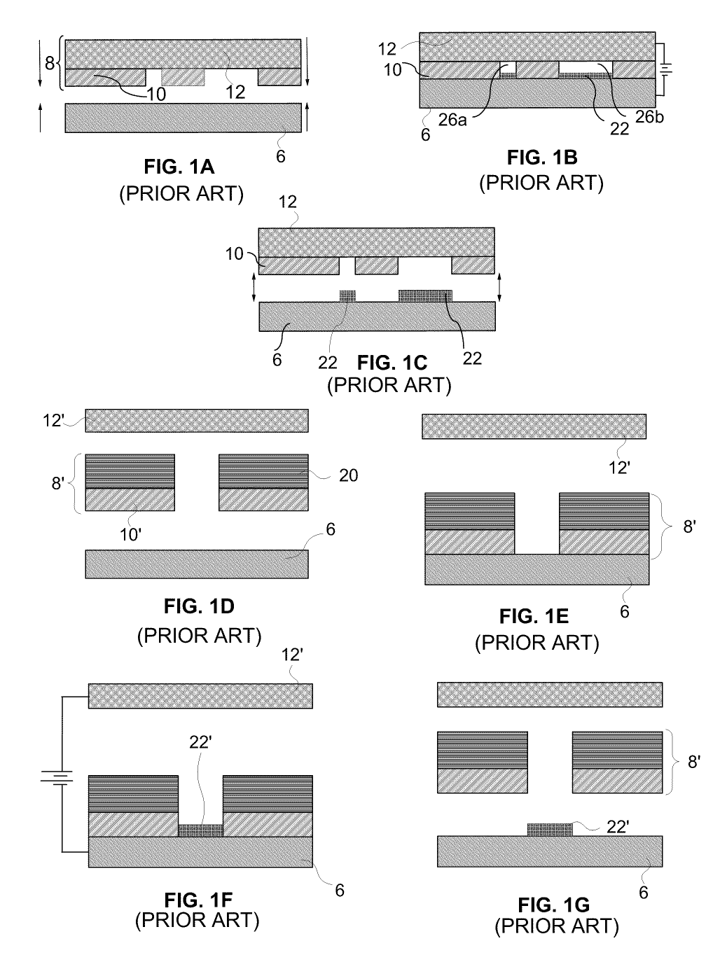

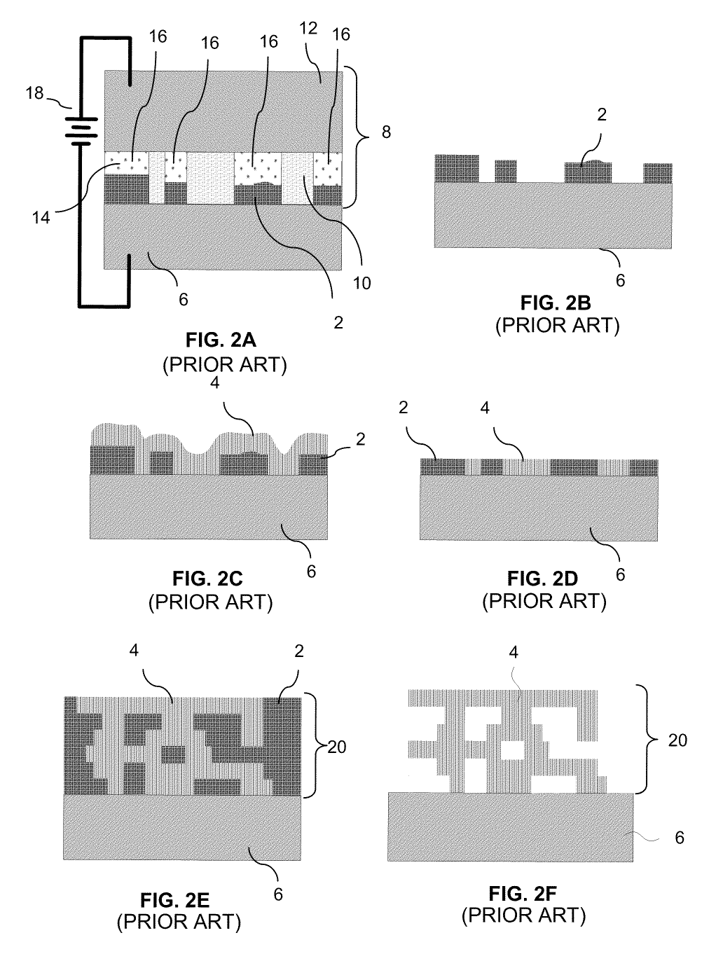

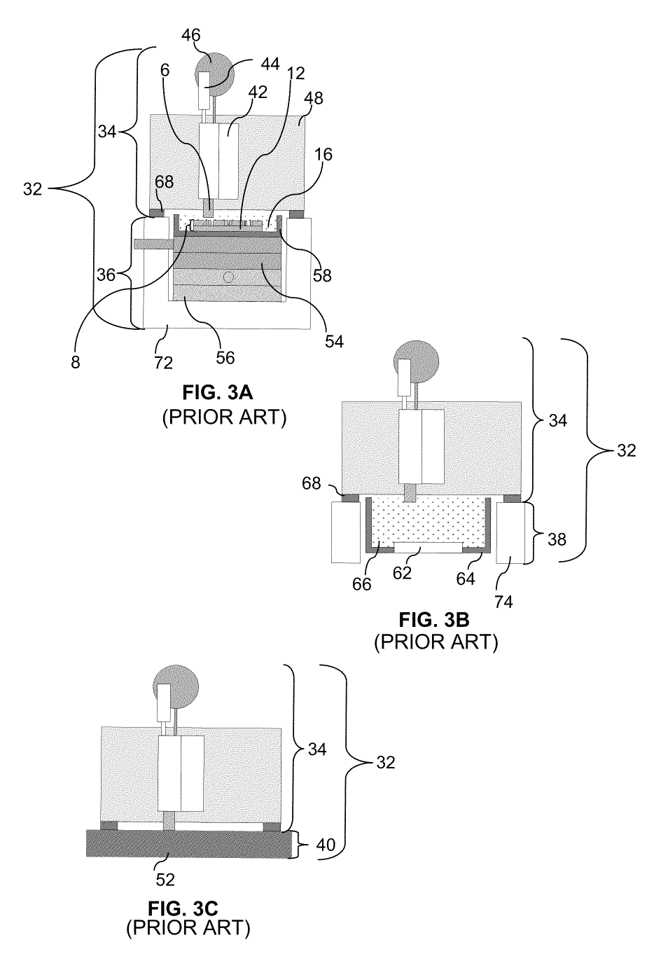

FIGS. 1A-1G, 2A-2F, and 3A-3C illustrate various features of one form of electrochemical fabrication. Other electrochemical fabrication techniques are set forth in the '630 patent referenced above, in the various previously incorporated publications, in various other patents and patent applications incorporated herein by reference. Still others may be derived from combinations of various approaches described in these publications, patents, and applications, or are otherwise known or ascertainable by those of skill in the art from the teachings set forth herein. All of these techniques may be combined with those of the various embodiments of various aspects of the invention to yield enhanced embodiments. Still other embodiments may be derived from combinations of the various embodiments explicitly set forth herein.

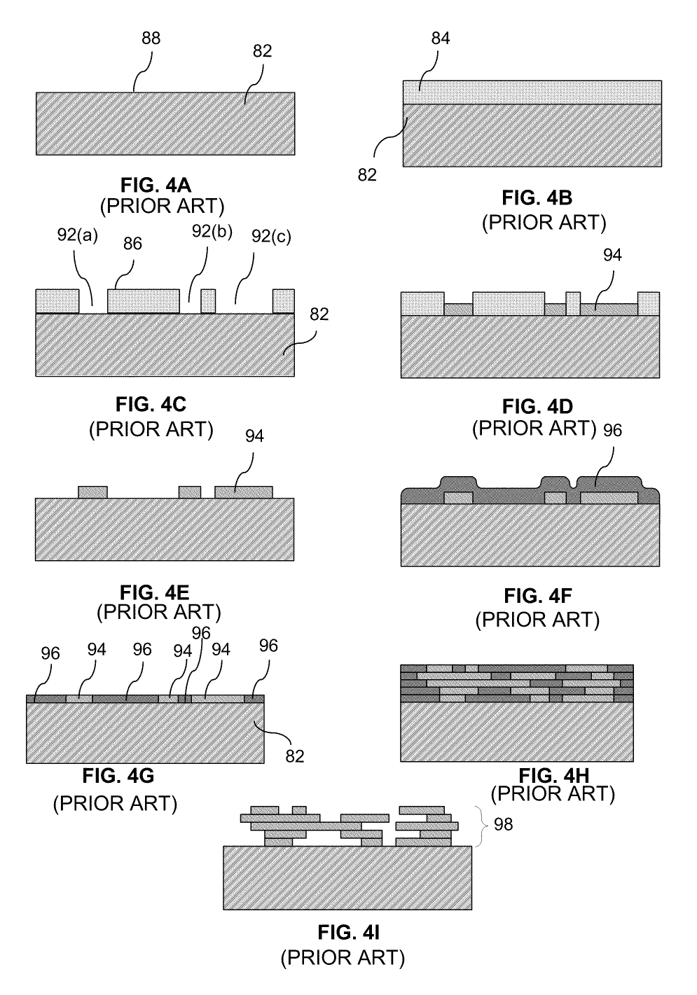

FIGS. 4A-4I illustrate various stages in the formation of a single layer of a multi-layer fabrication process where a second metal is deposited on a first metal as well as in openings in the first metal so that the first and second metal form part of the layer. In FIG. 4A a side view of a substrate 82 is shown, onto which patternable photoresist 84 is cast as shown in FIG. 4B. In FIG. 4C, a pattern of resist is shown that results from the curing, exposing, and developing of the resist. The patterning of the photoresist 84 results in openings or apertures 92(a)-92(c) extending from a surface 86 of the photoresist through the thickness of the photoresist to surface 88 of the substrate 82. In FIG. 4D a metal 94 (e.g. nickel) is shown as having been electroplated into the openings 92(a)-92(c). In FIG. 4E the photoresist has been removed (i.e. chemically stripped) from the substrate to expose regions of the substrate 82 which are not covered with the first metal 94. In FIG. 4F a second metal 96 (e.g. silver) is shown as having been blanket electroplated over the entire exposed portions of the substrate 82 (which is conductive) and over the first metal 94 (which is also conductive). FIG. 4G depicts the completed first layer of the structure which has resulted from the planarization of the first and second metals down to a height that exposes the first metal and sets a thickness for the first layer. In FIG. 4H the result of repeating the process steps shown in FIGS. 4B-4 G several times to form a multi-layer structure are shown where each layer consists of two materials. For most applications, one of these materials is removed as shown in FIG. 4I to yield a desired 3-D structure 98 (e.g. component or device).

Various embodiments of various aspects of the invention are directed to formation of three-dimensional structures from materials some of which may be electrodeposited or electroless deposited. Some of these structures may be formed form a single build level formed from one or more deposited materials while others are formed from a plurality of build layers each including at least two materials (e.g. two or more layers, more preferably five or more layers, and most preferably ten or more layers). In some embodiments, layer thicknesses may be as small as one micron or as large as fifty microns. In other embodiments, thinner layers may be used while in other embodiments, thicker layers may be used. In some embodiments structures having features positioned with micron level precision and minimum features size on the order of tens of microns are to be formed. In other embodiments structures with less precise feature placement and/or larger minimum features may be formed. In still other embodiments, higher precision and smaller minimum feature sizes may be desirable. In the present application meso-scale and millimeter scale have the same meaning and refer to devices that may have one or more dimensions extending into the 0.5-20 millimeter range, or somewhat larger and with features positioned with precision in the 10-100 micron range and with minimum features sizes on the order of 100 microns.

The various embodiments, alternatives, and techniques disclosed herein may form multi-layer structures using a single patterning technique on all layers or using different patterning techniques on different layers. For example, Various embodiments of the invention may perform selective patterning operations using conformable contact masks and masking operations (i.e. operations that use masks which are contacted to but not adhered to a substrate), proximity masks and masking operations (i.e. operations that use masks that at least partially selectively shield a substrate by their proximity to the substrate even if contact is not made), non-conformable masks and masking operations (i.e. masks and operations based on masks whose contact surfaces are not significantly conformable), and/or adhered masks and masking operations (masks and operations that use masks that are adhered to a substrate onto which selective deposition or etching is to occur as opposed to only being contacted to it). Conformable contact masks, proximity masks, and non-conformable contact masks share the property that they are preformed and brought to, or in proximity to, a surface which is to be treated (i.e. the exposed portions of the surface are to be treated). These masks can generally be removed without damaging the mask or the surface that received treatment to which they were contacted, or located in proximity to. Adhered masks are generally formed on the surface to be treated (i.e. the portion of that surface that is to be masked) and bonded to that surface such that they cannot be separated from that surface without being completely destroyed damaged beyond any point of reuse. Adhered masks may be formed in a number of ways including (1) by application of a photoresist, selective exposure of the photoresist, and then development of the photoresist, (2) selective transfer of pre-patterned masking material, and/or (3) direct formation of masks from computer controlled depositions of material.

Patterning operations may be used in selectively depositing material and/or may be used in the selective etching of material. Selectively etched regions may be selectively filled in or filled in via blanket deposition, or the like, with a different desired material. In some embodiments, the layer-by-layer build up may involve the simultaneous formation of portions of multiple layers. In some embodiments, depositions made in association with some layer levels may result in depositions to regions associated with other layer levels (i.e. regions that lie within the top and bottom boundary levels that define a different layer's geometric configuration). Such use of selective etching and interlaced material deposition in association with multiple layers is described in U.S. patent application Ser. No. 10/434,519, by Smalley, now U.S. Pat. No. 7,252,861, and entitled “Methods of and Apparatus for Electrochemically Fabricating Structures Via Interlaced Layers or Via Selective Etching and Filling of Voids layer elements” which is hereby incorporated herein by reference as if set forth in full.

Temporary substrates on which structures may be formed may be of the sacrificial-type (i.e. destroyed or damaged during separation of deposited materials to the extent they can not be reused), non-sacrificial-type (i.e. not destroyed or excessively damaged, i.e. not damaged to the extent they may not be reused, e.g. with a sacrificial or release layer located between the substrate and the initial layers of a structure that is formed). Non-sacrificial substrates may be considered reusable, with little or no rework (e.g. replanarizing one or more selected surfaces or applying a release layer, and the like) though they may or may not be reused for a variety of reasons.

### DEFINITIONS

This section of the specification is intended to set forth definitions for a number of specific terms that may be useful in describing the subject matter of the various embodiments of the invention. It is believed that the meanings of most if not all of these terms is clear from their general use in the specification but they are set forth hereinafter to remove any ambiguity that may exist. It is intended that these definitions be used in understanding the scope and limits of any claims that use these specific terms. As far as interpretation of the claims of this patent disclosure are concerned, it is intended that these definitions take presence over any contradictory definitions or allusions found in any materials which are incorporated herein by reference.

“Build” as used herein refers, as a verb, to the process of building a desired structure or plurality of structures from a plurality of applied or deposited materials which are stacked and adhered upon application or deposition or, as a noun, to the physical structure or structures formed from such a process. Depending on the context in which the term is used, such physical structures may include a desired structure embedded within a sacrificial material or may include only desired physical structures which may be separated from one another or may require dicing and/or slicing to cause separation.

“Build axis” or “build orientation” is the axis or orientation that is substantially perpendicular to substantially planar levels of deposited or applied materials that are used in building up a structure. The planar levels of deposited or applied materials may be or may not be completely planar but are substantially so in that the overall extent of their cross-sectional dimensions are significantly greater than the height of any individual deposit or application of material (e.g. 100, 500, 1000, 5000, or more times greater). The planar nature of the deposited or applied materials may come about from use of a process that leads to planar deposits or it may result from a planarization process (e.g. a process that includes mechanical abrasion, e.g. lapping, fly cutting, grinding, or the like) that is used to remove material regions of excess height. Unless explicitly noted otherwise, “vertical” as used herein refers to the build axis or nominal build axis (if the layers are not stacking with perfect registration) while “horizontal” refers to a direction within the plane of the layers (i.e. the plane that is substantially perpendicular to the build axis).

“Build layer” or “layer of structure” as used herein does not refer to a deposit of a specific material but instead refers to a region of a build located between a lower boundary level and an upper boundary level which generally defines a single cross-section of a structure being formed or structures which are being formed in parallel. Depending on the details of the actual process used to form the structure, build layers are generally formed on and adhered to previously formed build layers. In some processes the boundaries between build layers are defined by planarization operations which result in successive build layers being formed on substantially planar upper surfaces of previously formed build layers. In some embodiments, the substantially planar upper surface of the preceding build layer may be textured to improve adhesion between the layers. In other build processes, openings may exist in or be formed in the upper surface of a previous but only partially formed build layers such that the openings in the previous build layers are filled with materials deposited in association with current build layers which will cause interlacing of build layers and material deposits. Such interlacing is described in U.S. patent application Ser. No. 10/434,519, now U.S. Pat. No. 7,252,861. This referenced application is incorporated herein by reference as if set forth in full. In most embodiments, a build layer includes at least one primary structural material and at least one primary sacrificial material. However, in some embodiments, two or more primary structural materials may used without a primary sacrificial material (e.g. when one primary structural material is a dielectric and the other is a conductive material). In some embodiments, build layers are distinguishable from each other by the source of the data that is used to yield patterns of the deposits, applications, and/or etchings of material that form the respective build layers. For example, data descriptive of a structure to be formed which is derived from data extracted from different vertical levels of a data representation of the structure define different build layers of the structure. The vertical separation of successive pairs of such descriptive data may define the thickness of build layers associated with the data. As used herein, at times, “build layer” may be loosely referred simply as “layer”. In many embodiments, deposition thickness of primary structural or sacrificial materials (i.e. the thickness of any particular material after it is deposited) is generally greater than the layer thickness and a net deposit thickness is set via one or more planarization processes which may include, for example, mechanical abrasion (e.g. lapping, fly cutting, polishing, and the like) and/or chemical etching (e.g. using selective or non-selective etchants). The lower boundary and upper boundary for a build layer may be set and defined in different ways. From a design point of view they may be set based on a desired vertical resolution of the structure (which may vary with height). From a data manipulation point of view, the vertical layer boundaries may be defined as the vertical levels at which data descriptive of the structure is processed or the layer thickness may be defined as the height separating successive levels of cross-sectional data that dictate how the structure will be formed. From a fabrication point of view, depending on the exact fabrication process used, the upper and lower layer boundaries may be defined in a variety of different ways. For example by planarization levels or effective planarization levels (e.g. lapping levels, fly cutting levels, chemical mechanical polishing levels, mechanical polishing levels, vertical positions of structural and/or sacrificial materials after relatively uniform etch back following a mechanical or chemical mechanical planarization process). For example, by levels at which process steps or operations are repeated. At levels at which, at least theoretically, lateral extends of structural material can be changed to define new cross-sectional features of a structure.

“Layer thickness” is the height along the build axis between a lower boundary of a build layer and an upper boundary of that build layer.

“Planarization” is a process that tends to remove materials, above a desired plane, in a substantially non-selective manner such that all deposited materials are brought to a substantially common height or desired level (e.g. within 20%, 10%, 5%, or even 1% of a desired layer boundary level). For example, lapping removes material in a substantially non-selective manner though some amount of recession one material or another may occur (e.g. copper may recess relative to nickel). Planarization may occur primarily via mechanical means, e.g. lapping, grinding, fly cutting, milling, sanding, abrasive polishing, frictionally induced melting, other machining operations, or the like (i.e. mechanical planarization). Mechanical planarization maybe followed or proceeded by thermally induced planarization (e.g. melting) or chemically induced planarization (e.g. etching). Planarization may occur primarily via a chemical and/or electrical means (e.g. chemical etching, electrochemical etching, or the like). Planarization may occur via a simultaneous combination of mechanical and chemical etching (e.g. chemical mechanical polishing (CMP)).

“Structural material” as used herein refers to a material that remains part of the structure when put into use.

“Supplemental structural material” as used herein refers to a material that forms part of the structure when the structure is put to use but is not added as part of the build layers but instead is added to a plurality of layers simultaneously (e.g. via one or more coating operations that applies the material, selectively or in a blanket fashion, to a one or more surfaces of a desired build structure that has been released from a sacrificial material.

“Primary structural material” as used herein is a structural material that forms part of a given build layer and which is typically deposited or applied during the formation of that build layer and which makes up more than 20% of the structural material volume of the given build layer. In some embodiments, the primary structural material may be the same on each of a plurality of build layers or it may be different on different build layers. In some embodiments, a given primary structural material may be formed from two or more materials by the alloying or diffusion of two or more materials to form a single material.

“Secondary structural material” as used herein is a structural material that forms part of a given build layer and is typically deposited or applied during the formation of the given build layer but is not a primary structural material as it individually accounts for only a small volume of the structural material associated with the given layer. A secondary structural material will account for less than 20% of the volume of the structural material associated with the given layer. In some preferred embodiments, each secondary structural material may account for less than 10%, 5%, or even 2% of the volume of the structural material associated with the given layer. Examples of secondary structural materials may include seed layer materials, adhesion layer materials, barrier layer materials (e.g. diffusion barrier material), and the like. These secondary structural materials are typically applied to form coatings having thicknesses less than 2 microns, 1 micron, 0.5 microns, or even 0.2 microns). The coatings may be applied in a conformal or directional manner (e.g. via CVD, PVD, electroless deposition, or the like). Such coatings may be applied in a blanket manner or in a selective manner. Such coatings may be applied in a planar manner (e.g. over previously planarized layers of material) as taught in U.S. patent application Ser. No. 10/607,931, now U.S. Pat. No. 7,239,219. In other embodiments, such coatings may be applied in a non-planar manner, for example, in openings in and over a patterned masking material that has been applied to previously planarized layers of material as taught in U.S. patent application Ser. No. 10/841,383, now U.S. Pat. No. 7,195,989. These referenced applications are incorporated herein by reference as if set forth in full herein.

“Functional structural material” as used herein is a structural material that would have been removed as a sacrificial material but for its actual or effective encapsulation by other structural materials. Effective encapsulation refers, for example, to the inability of an etchant to attack the functional structural material due to inaccessibility that results from a very small area of exposure and/or due to an elongated or tortuous exposure path. For example, large (10,000 μm²) but thin (e.g. less than 0.5 microns) regions of sacrificial copper sandwiched between deposits of nickel may define regions of functional structural material depending on ability of a release etchant to remove the sandwiched copper.

“Sacrificial material” is material that forms part of a build layer but is not a structural material. Sacrificial material on a given build layer is separated from structural material on that build layer after formation of that build layer is completed and more generally is removed from a plurality of layers after completion of the formation of the plurality of layers during a “release” process that removes the bulk of the sacrificial material or materials. In general sacrificial material is located on a build layer during the formation of one, two, or more subsequent build layers and is thereafter removed in a manner that does not lead to a planarized surface. Materials that are applied primarily for masking purposes, i.e. to allow subsequent selective deposition or etching of a material, e.g. photoresist that is used in forming a build layer but does not form part of the build layer) or that exist as part of a build for less than one or two complete build layer formation cycles are not considered sacrificial materials as the term is used herein but instead shall be referred as masking materials or as temporary materials. These separation processes are sometimes referred to as a release process and may or may not involve the separation of structural material from a build substrate. In many embodiments, sacrificial material within a given build layer is not removed until all build layers making up the three-dimensional structure have been formed. Of course sacrificial material may be, and typically is, removed from above the upper level of a current build layer during planarization operations during the formation of the current build layer. Sacrificial material is typically removed via a chemical etching operation but in some embodiments may be removed via a melting operation or electrochemical etching operation. In typical structures, the removal of the sacrificial material (i.e. release of the structural material from the sacrificial material) does not result in planarized surfaces but instead results in surfaces that are dictated by the boundaries of structural materials located on each build layer. Sacrificial materials are typically distinct from structural materials by having different properties therefrom (e.g. chemical etchability, hardness, melting point, etc.) but in some cases, as noted previously, what would have been a sacrificial material may become a structural material by its actual or effective encapsulation by other structural materials. Similarly, structural materials may be used to form sacrificial structures that are separated from a desired structure during a release process via the sacrificial structures being only attached to sacrificial material or potentially by dissolution of the sacrificial structures themselves using a process that is insufficient to reach structural material that is intended to form part of a desired structure. It should be understood that in some embodiments, small amounts of structural material may be removed, after or during release of sacrificial material. Such small amounts of structural material may have been inadvertently formed due to imperfections in the fabrication process or may result from the proper application of the process but may result in features that are less than optimal (e.g. layers with stairs steps in regions where smooth sloped surfaces are desired. In such cases the volume of structural material removed is typically minuscule compared to the amount that is retained and thus such removal is ignored when labeling materials as sacrificial or structural. Sacrificial materials are typically removed by a dissolution process, or the like, that destroys the geometric configuration of the sacrificial material as it existed on the build layers. In many embodiments, the sacrificial material is a conductive material such as a metal. As will be discussed hereafter, masking materials though typically sacrificial in nature are not termed sacrificial materials herein unless they meet the required definition of sacrificial material.

“Supplemental sacrificial material” as used herein refers to a material that does not form part of the structure when the structure is put to use and is not added as part of the build layers but instead is added to a plurality of layers simultaneously (e.g. via one or more coating operations that applies the material, selectively or in a blanket fashion, to a one or more surfaces of a desired build structure that has been released from an initial sacrificial material. This supplemental sacrificial material will remain in place for a period of time and/or during the performance of certain post layer formation operations, e.g. to protect the structure that was released from a primary sacrificial material, but will be removed prior to putting the structure to use.

“Primary sacrificial material” as used herein is a sacrificial material that is located on a given build layer and which is typically deposited or applied during the formation of that build layer and which makes up more than 20% of the sacrificial material volume of the given build layer. In some embodiments, the primary sacrificial material may be the same on each of a plurality of build layers or may be different on different build layers. In some embodiments, a given primary sacrificial material may be formed from two or more materials by the alloying or diffusion of two or more materials to form a single material.

“Secondary sacrificial material” as used herein is a sacrificial material that is located on a given build layer and is typically deposited or applied during the formation of the build layer but is not a primary sacrificial materials as it individually accounts for only a small volume of the sacrificial material associated with the given layer. A secondary sacrificial material will account for less than 20% of the volume of the sacrificial material associated with the given layer. In some preferred embodiments, each secondary sacrificial material may account for less than 10%, 5%, or even 2% of the volume of the sacrificial material associated with the given layer. Examples of secondary structural materials may include seed layer materials, adhesion layer materials, barrier layer materials (e.g. diffusion barrier material), and the like. These secondary sacrificial materials are typically applied to form coatings having thicknesses less than 2 microns, 1 micron, 0.5 microns, or even 0.2 microns). The coatings may be applied in a conformal or directional manner (e.g. via CVD, PVD, electroless deposition, or the like). Such coatings may be applied in a blanket manner or in a selective manner. Such coatings may be applied in a planar manner (e.g. over previously planarized layers of material) as taught in U.S. patent application Ser. No. 10/607,931, now U.S. Pat. No. 7,239,219. In other embodiments, such coatings may be applied in a non-planar manner, for example, in openings in and over a patterned masking material that has been applied to previously planarized layers of material as taught in U.S. patent application Ser. No. 10/841,383, now U.S. Pat. No. 7,195,989. These referenced applications are incorporated herein by reference as if set forth in full herein.

“Adhesion layer”, “seed layer”, “barrier layer”, and the like refer to coatings of material that are thin in comparison to the layer thickness and thus generally form secondary structural material portions or sacrificial material portions of some layers. Such coatings may be applied uniformly over a previously formed build layer, they may be applied over a portion of a previously formed build layer and over patterned structural or sacrificial material existing on a current (i.e. partially formed) build layer so that a non-planar seed layer results, or they may be selectively applied to only certain locations on a previously formed build layer. In the event such coatings are non-selectively applied, selected portions may be removed (1) prior to depositing either a sacrificial material or structural material as part of a current layer or (2) prior to beginning formation of the next layer or they may remain in place through the layer build up process and then etched away after formation of a plurality of build layers.

“Masking material” is a material that may be used as a tool in the process of forming a build layer but does not form part of that build layer. Masking material is typically a photopolymer or photoresist material or other material that may be readily patterned. Masking material is typically a dielectric. Masking material, though typically sacrificial in nature, is not a sacrificial material as the term is used herein. Masking material is typically applied to a surface during the formation of a build layer for the purpose of allowing selective deposition, etching, or other treatment and is removed either during the process of forming that build layer or immediately after the formation of that build layer.

“Multilayer structures” are structures formed from multiple build layers of deposited or applied materials.

“Multilayer three-dimensional (or 3D or 3-D) structures” are Multilayer Structures that meet at least one of two criteria: (1) the structural material portion of at least two layers of which one has structural material portions that do not overlap structural material portions of the other.

“Complex multilayer three-dimensional (or 3D or 3-D) structures” are multilayer three-dimensional structures formed from at least three layers where a line may be defined that hypothetically extends vertically through at least some portion of the build layers of the structure will extend from structural material through sacrificial material and back through structural material or will extend from sacrificial material through structural material and back through sacrificial material (these might be termed vertically complex multilayer three-dimensional structures). Alternatively, complex multilayer three-dimensional structures may be defined as multilayer three-dimensional structures formed from at least two layers where a line may be defined that hypothetically extends horizontally through at least some portion of a build layer of the structure that will extend from structural material through sacrificial material and back through structural material or will extend from sacrificial material through structural material and back through sacrificial material (these might be termed horizontally complex multilayer three-dimensional structures). Worded another way, in complex multilayer three-dimensional structures, a vertically or horizontally extending hypothetical line will extend from one or structural material or void (when the sacrificial material is removed) to the other of void or structural material and then back to structural material or void as the line is traversed along at least a portion of the line.

“Moderately complex multilayer three-dimensional (or 3D or 3-D) structures are complex multilayer 3D structures for which the alternating of void and structure or structure and void not only exists along one of a vertically or horizontally extending line but along lines extending both vertically and horizontally.

“Highly complex multilayer (or 3D or 3-D) structures are complex multilayer 3D structures for which the structure-to-void-to-structure or void-to-structure-to-void alternating occurs once along the line but occurs a plurality of times along a definable horizontally or vertically extending line.

“Up-facing feature” is an element dictated by the cross-sectional data for a given build layer “n” and a next build layer “n+1” that is to be formed from a given material that exists on the build layer “n” but does not exist on the immediately succeeding build layer “n+1”. For convenience the term “up-facing feature” will apply to such features regardless of the build orientation.

“Down-facing feature” is an element dictated by the cross-sectional data for a given build layer “n” and a preceding build layer “n−1” that is to be formed from a given material that exists on build layer “n” but does not exist on the immediately preceding build layer “n−1”. As with up-facing features, the term “down-facing feature” shall apply to such features regardless of the actual build orientation.

“Continuing region” is the portion of a given build layer “n” that is dictated by the cross-sectional data for the given build layer “n”, a next build layer “n+1” and a preceding build layer “n−1” that is neither up-facing nor down-facing for the build layer “n”.

“Minimum feature size” refers to a necessary or desirable spacing between structural material elements on a given layer that are to remain distinct in the final device configuration. If the minimum feature size is not maintained on a given layer, the fabrication process may result in structural material inadvertently bridging the two structural elements due to masking material failure or failure to appropriately fill voids with sacrificial material during formation of the given layer such that during formation of a subsequent layer structural material inadvertently fills the void. More care during fabrication can lead to a reduction in minimum feature size or a willingness to accept greater losses in productivity can result in a decrease in the minimum feature size. However, during fabrication for a given set of process parameters, inspection diligence, and yield (successful level of production) a minimum design feature size is set in one way or another. The above described minimum feature size may more appropriately be termed minimum feature size of sacrificial material regions. Conversely a minimum feature size for structure material regions (minimum width or length of structural material elements) may be specified. Depending on the fabrication method and order of deposition of structural material and sacrificial material, the two types of minimum feature sizes may be different. In practice, for example, using electrochemical fabrication methods and described herein, the minimum features size on a given layer may be roughly set to a value that approximates the layer thickness used to form the layer and it may be considered the same for both structural and sacrificial material widths and lengths. In some more rigorously implemented processes, examination regiments, and rework requirements, it may be set to an amount that is 80%, 50%, or even 30% of the layer thickness. Other values or methods of setting minimum feature sizes may be set.

“Sublayer” as used herein refers to a portion of a build layer that typically includes the full lateral extents of that build layer but only a portion of its height. A sublayer is usually a vertical portion of build layer that undergoes independent processing compared to another sublayer of that build layer.

Intradermal and/or Transdermal Needle Devices

Numerous embodiments of the present invention exist. Some embodiments are directed to methods for applying drugs, others are directed to fabricating devices that are used for applying drugs, while still others are directed to the drug delivery devices themselves. In some embodiments the devices may be used by patients, while in other embodiments the devices may be operated by medical professionals for the benefit of patients. In some embodiments drugs delivered by the devices may provide therapeutic treatment while in others the treatments may be preventive in nature, and instill others they may be for diagnostic purposes. In some embodiments, the use of distributed application of drugs may provide local treatment to broad areas, reduced pain associated with drug delivery, faster uptake of the drug, and/or less local area irritation associated with concentrated drug delivery.

Structural Features:

In a first group of embodiments, a plurality of needles are located on the outer surface of a cylindrical body that may be rolled along a surface to be treated with a drug such that as the cylindrical surface is rotated a portion of the needles are inserted through the surface of the skin to a depth corresponding to a desired delivery or extraction depth while others are removed from the skin. The spacing between adjacent needles along the length of the cylindrical body and the spacing along the circumference of the cylindrical body may be selected based on drug delivery requirements, fluid extraction requirements, or based on other requirements or desires (e.g. manufacturing restrictions, redundancy requirements, and the like). The needles of the first group of embodiments preferable are of the non-coring type and include at least one distal or near-distal aperture connected to a cannula which in turn connects to a drug supply reservoir or to an extraction chamber. However, in some embodiments, the needles may include a slot or other external channel along with a drug or other fluid that may be transported. In some embodiments, though not preferred, the needles may not be of the non-coring type.

In one group of variations of the first group of embodiments, a plurality of needles are located such that they extend fixedly through a surface of a cylindrical body. In another group of variations of the first group of embodiments, a plurality of needles are configured and mounted with respect to the cylindrically body such that they can be made to extend periodically and controllably through the cylindrical body.

In a second group of embodiments, the cylindrical configuration of the first embodiment and its variations is replaced by a non-cylindrical body that has a smooth contact surface that may have a fixed configuration or may have a deformable or variable configuration.

In a third group of embodiments the cylindrical body or the non-cylindrical body of the first and second groups of embodiments, respectively, may be replaced by a plurality of adjacent ring-like bodies or loop-bodies that may be made to extend or retract their respective contact surfaces relative to adjacent rings or loops to ensure mating contact with local portions of a delivery surface.

In a forth group of embodiments, the needles of the first-third groups of embodiments include base elements that provide penetration stops (i.e. mating surfaces) so that penetration depth is limited to a desired amount.

In a fifth group of embodiments, a plurality of needles are located on the outer surface of a cylindrical body or smooth body (e.g. in a single row or in a plurality of closely spaced rows) but instead of the body rolling along a surface to be treated the body slides along the surface to be treated and the needles are periodically extended from the body to pierce the surface to a predefined depth, dispense the drug and then to retract back into the body. In variations of this embodiment, needle extensions and delivery may occur manually by operator interaction (e.g. pulling a trigger or pushing a button) wherein the quantity delivered per needle per actuation is a (1) constant, (2) a variable that is controlled, for example, by the hold time of the actuator, (3) a variable that can be set by other mechanisms or control elements found with the delivery system, or (4) a variable that is set based on automatic recognition of a drug type or drug package type this is loaded into the delivery device. In some embodiments one or more rotating elements or other movement sensing elements may located on the slider so that an amount of translational movement may be detected and automatic needle displacement and actuation made to occur at regular and predetermined intervals along a target surface. In some embodiments, this predetermined interval may be a variable that is set directly or indirectly by a user, e.g. by setting a distance control variable or by setting a medication type, or automatically by the device, e.g. by detection of a type of medication that has been loaded into the device.

Sixth-eight embodiments of the invention are similar to the second-fourth embodiments of the invention, mutatis mutandis, with the primary exception that instead of the cylindrical or smooth body a rolling, its slides as was the case with the fifth embodiment.

In variations of the first-eighth groups of embodiments, the plurality of needles in a give device may all have common configurations and orientations or they may have different configurations and/or orientations. In some variations, some needles may extend further than others (i.e. have different lengths). In some variations the needle extension length may be a fixed amount for a given device, may be a variable amount that is user selectable, e.g. by electronic circuitry, programming, or mechanical control (e.g. selecting a particular needle extension cam or set of cams for use in a particular application).

In some further variations of the first-eighth embodiments, all the needles may be connected to a common drug supply while in other variations, the needles may have their own drug reservoirs, while in still other embodiments, groups of needles may be connected to different reservoirs and in such variations different drugs or different drug parts may be dispensed by different needles or different groups of needles.

In some variations of the first-eighth embodiments, features may be added to the device to limit the dispensing of drugs to a fractional portion of the needles. In a first group of such variations, fluid flow or drug flow from needles may be limited to those needles that have penetrated the skin or delivery surface. In other embodiments, fluid flow from individual non-selected needles may be reduced to less than 20%, more preferable less than 10%, even more preferably less than 5%, and most preferably less than 1% of that from selected needles. Means for limiting such flow may be external to the cylindrical or smooth body, or may be internal to the body. Such means, for example, may include one or more of:
(1) membranes which are only penetrated by the needles when the relevant part of the cylindrical surface is made to contact a delivery surface;
(2) a rotating shield located on the interior surface of the cylindrical body or smooth body that shields access to the needle lumens from a reservoir of the drug with the exception of those needles that are located along a desired flow path (e.g. those needles that are inserted into the skin);
(3) a sponge-like material acting as a reservoir for the drug to be dispensed located against the interior surface of the cylindrical body or the smooth body and a cam or cam like structure which presses and squeezes material from the pores of the sponge at the location or locations where the extended portion of the cam contacts the sponge-like material (e.g. in locations coordinated to be over the needles which have penetrated into the body of the patient) in some alternative embodiments such cam like structure may also be used to drive the needles out of the cylindrical body so that they may extend a desired amount from the body and/or so that they may penetrate a sealing membrane and thereafter the target surface;
(4) individual needles or groups of needles may be normally sealed (e.g. by spring loaded sealing materials and which may made to open to allow flow in response, for example, to (a) mechanical interaction with lifting elements (e.g. inward bulging rails, and the like), (b) magnetic forces, (c) electrostatic forces, and/or (d) vacuum forces.

In some variations of these embodiments, the needles may function as electrodes as well as delivery or uptake ports and/or other electrodes may be provided between some of the needles wherein electrical conductive is used as a delivery trigger that opens separate valve elements or functions directly as a flow enhancer (e.g. for iontophoretic delivery, via voltage or current enhanced fluid flow, or the like). In some embodiments, electrical conductivity associated with different needles relative to other portions of the same needles, or relative to different needles, may be used to ascertain the quality of tissue contact or even depth of tissue penetration. Motivation to extend needles and/or to allow flow may be achieved by, for example, mechanical stimulation associated with rolling the cylindrical or smooth body along a dispensing surface or by electrical motivation in the form of batteries which are located in the device. Triggers for extending needles and/or to cause flow may be supplied by pure mechanical positioning or by sensors or the like (e.g. pressure sensors, conductivity sensors, light sensors, proximity sensors, etc.).

In some variations of each of the above embodiments, the cylindrical or smooth body may include features for guiding relatively long needles so that they do not inadvertently bend during insertion. In still other variations such relatively long needles may include catches or other features, located between distal and proximal ends, which may be engaged by triggering devices to allow movement of the needles without necessarily needing to transmit a force from the proximal end of the needle to the distal end. Such intermediate features could reduce the likelihood of bending during insertion.

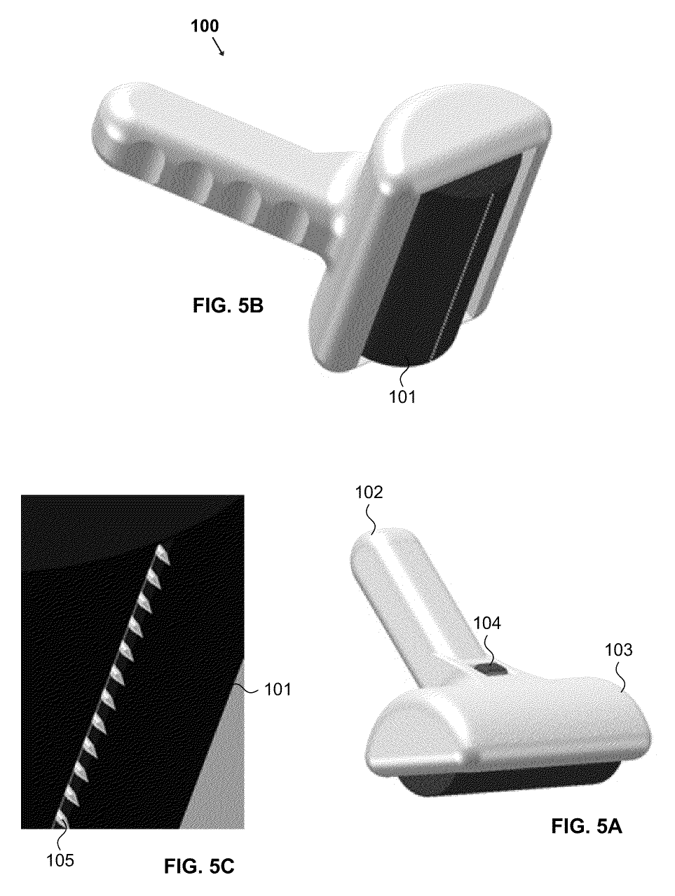

FIGS. 5A and 5B provide perspective views of a device 100 according to some embodiments of the invention. The device including a handle 102, a housing 103, an optional trigger button 104 and associated mechanisms and/or control electronics (not shown), e.g. for extending needles and dispensing material, and a slide (e.g. the fifth group of embodiments) or cylindrical body (e.g. first group of embodiments) 101 on which one or more linear needle arrays 105 are mounted or from which they are extendable and which are capable of dispensing or extracting a desired drug or fluid material. As shown, in FIGS. 5A and 5B, a single linear array of needles is extended from the slide or cylindrical body. In the case of a sliding element this same array of needles may be extended a plurality of times in succession to deliver a material or to allow extraction of a material.

FIG. 5C provides a perspective view of a single linear needle array 105 extending from the slide or cylindrical body. During use the needles are retracted into the slide during movement across the delivery site and are extended from the slide or cylinder during dispensing times. In some uses, extension may be momentarily during constant movement or may occur only when the device becomes stationary. Detection of movement and or rate of movement may occur by an operator or by the device itself via one or more onboard sensors.

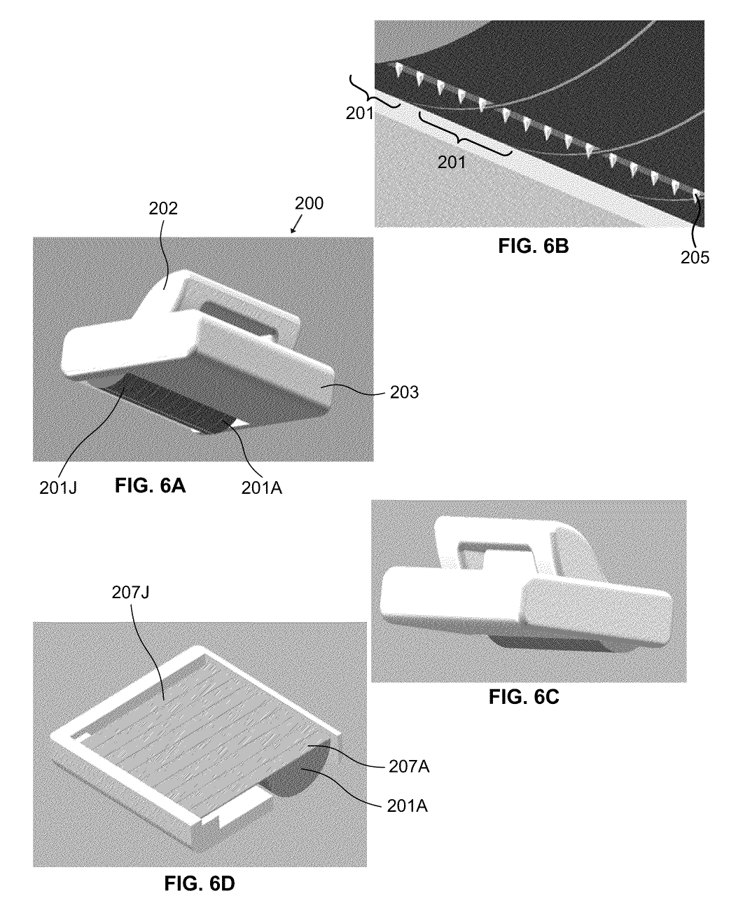

FIGS. 6A and 6B provide perspective views of a device 200 according to some embodiments of the invention (e.g. the third or seventh group of embodiments). The device includes a handle 202, a housing 203, and a plurality of independently supported slides or rings 201A-201J on which one or more needles are mounted or from which they are extendable to form a single linear needle array 205 and which are capable of dispensing or extracting a desired drug or fluid material.

FIG. 6C provides a perspective view of a single linear needle array 205 extending from the slides or rings 207A-207J. During use the needles may be retracted into the slide or rings during movement across the delivery site and may be extended from the slide or rings when during dispensing times. In some uses, extension may be momentarily during constant movement or may occur only when the device becomes stationary. Detection of movement and or rate of movement may occur by an operator or by the device itself via one or more onboard sensors.

FIG. 6D depicts additional elements found in the variation of the embodiments of FIGS. 6A-6C wherein a slider is used as opposed to rotatable rings. In these variations, independent leaf springs 207A-207J are included for biasing each of the slides to allow more intimate contact of the individual slider elements with the delivery site along the width of the needle array. Such leaf springs or other compliant elements may be used to bias the backsides or central axes of rotatable rings in embodiments that use them.

In the embodiments of FIGS. 6A-6C, and the variation of FIG. 6D, each slide includes one or more needles which can be recessed into and extended out of the slide and each rotatable ring preferably, but not necessarily, includes one or more needles which can be recessed into and extended out of the ring. The recession and extension may occur in a number of different ways including (1) manually by depressing a trigger which comprises or expands a spring (e.g. a bi-stable switch) which may regain its normal state by retriggering or time delay, for example, by (2) electrically activated solenoid, or (3) pneumatic actuation. The system may use batteries and include lights or other signaling mechanism that provide an indication as to the state of the needles (retracted or extended) and/or the state of dispensing or fluid extraction. The frequency of reciprocation (extension and recession) may be controlled manually or automatically by detection of changes in position (e.g. mechanically by rotating wheels, optically, electrically, magnetically, or the like).

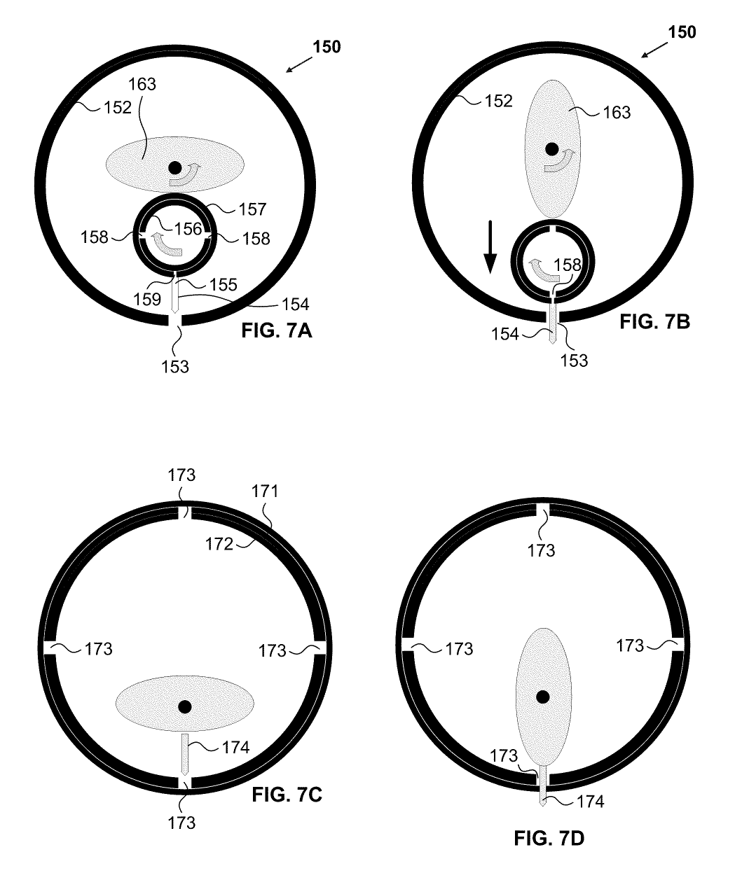

Examples of a two flow restriction and needle extension and retraction options are shown in FIGS. 7A-7D. The Example of FIGS. 7A-7B is most useful in a sliding embodiment thought it may be also be used in a rotating cylinder or ring embodiment. The example of FIGS. 7C-7D may be used in a rotating cylinder embodiment, a rotating ring embodiment, or in a sliding embodiment.

The device implementation 150 of FIGS. 7A-7B includes an outer slide 152 including a slot, or plurality of openings, 153 extending axially along the length of the outer slide. The opening or openings 153 allow needles 154 to be retracted into and extended from the outer slide. Needles 154 are functionally coupled to a pair of concentric cylinders 156 and 157 wherein the outer most of these cylinders 157 includes a plurality of openings 159 (e.g. a linear array) which functionally connects to the flow paths within needles 154. The inner most of these cylinders 156 rotates relative to the outer most cylinder 157 such that a slot or set of openings 158 in the inner cylinder periodically align with the openings 159 so that a material within the inner volume of cylinder 156 can flow out of needles 154 (FIG. 7B) via paths 155. On the other hand when the openings 158 and 159 are not aligned little (e.g. less than 10%, 1% or even 0.1% of flow rate when aligned) or no flow will occur. In the implementation of this example, while cylinder 157 rotates, an eccentric element 163 also rotates about a fixed axis 164 relative to slide 152 so that when alignment of openings 158 and 159 occurs, the needles 154 and cylinders 156 and 157 are made to move toward opening 153 such that needles 154 extend from opening 153 by a desired distance into a target surface against which slide 152 is pressed. Upon further relative rotation of eccentric 163 the needles are extracted from the target surface back inside of slider 152 while further rotation of inner cylinder 157 results in misalignment of openings 158 and 159 and associated blockage of flow paths 158-159-155. The devices of FIGS. 7A-7B also include various biasing elements (e.g. springs) for returning needles and associated cylindrical elements to their retracted positions

The device implementation 170 of FIGS. 7C-7D includes an outer slide or rotating cylinder 172 having one or more openings or arrays of openings 173. On the outside of the slide or cylinder 172 is a self sealing membrane (e.g. polymer or other material) through which needles 174 can periodically extend. In some alternative embodiments, the self sealing polymer may be located on the inside of cylinder 172 or be sandwiched between two cylindrical elements that together form the slide of cylinder 172. The slot, or plurality of openings, 173 extend axially along the working length of the outer slide and multiple such slots or sets of opening can exist around the periphery of the cylinder 172. The opening or openings 173 allow needles 174 to be retracted into and extended from the outer slide or cylinder. As such extension or retraction occurs, the self sealing barrier is penetrated or closed, respectively. Needles 174 are functionally coupled to move in and out of cylinder or slide 172 via rotation of an eccentric element 163 which rotates about a fixed axis 174 relative to slide or cylinder 172. In this embodiment, fluid pumping, or pressure to cause fluid flow may occur continuously or at discrete intervals. Fluid flow (e.g. drug flow) will only be effective to leave slider or cylinder 172 when the needles have penetrated the self sealing membrane. In some implementations, fluid flow may not be initiated until the needles have extended a particular distance from the membrane and may be stopped prior to extracting the needles back into the inside of the cylinder. The devices of FIGS. 7C-7D also include various biasing elements (e.g. springs) for returning needles and associated cylindrical elements to their retracted positions

In some implementations, the outer cylinder may include a plurality of openings opening arrays 173 so that has it rolls across a surface to be treated, the needles may be extended from different openings thus allowing two potential benefits: (1) decoupling of the cylinder diameter from locations of delivery as movement along the target surface occurs and (2) allowing different portions of the self sealing membrane to be penetrated thus potentially extending the functional life of the membrane. In some variations of this implementation multiple linear arrays of needles may be rotated into position as for dispensing by locating the needles on a cylinder similar to that of the implementation of FIGS. 7A-7B wherein successive arrays of needles may have different longitudinal positions different numbers, different lengths, different diameters, connections to different materials to be dispensed, or the like.

In variations of the embodiments illustrated in FIGS. 5A-5C, 6A-6D, and 7A-7D, different numbers of needles may be present. The needles may be located in the device along a single linear array, be located in a plurality of linear arrays that extend one at a time from the cylindrical body, rings, slider, or sliders, or in groups of linear arrays. In some embodiments the needles may be located in two dimensional arrays of various configurations.

Functional Features:

Needles are configured to have a penetration depth preferably in the range of 100 microns to 2 millimeters, more preferably in the range of 100-400 microns, in even more particularly in a range of 100-350 microns. Of course in other embodiments, the range may extend to shallower depths or even deeper depths. In some preferred embodiments the needles have a penetration depth that is limited to less than that which would reach nerve ends and/or blood vessels but great enough to allow the drug to readily enter the capillaries. The penetration depth may be varied depending on the type of skin being penetrated, the type of drug being delivered, the delivery rate being used, and/or the intended goal of treatment.

Needles may be configured to have a spacing from neighboring needles which is 1 mm, or less, to 10 mm, or more, more preferably between 1 mm and 5 mm and even more preferably between 1 mm and 3 mm.

Needles are configured to provide dispensing to a width in the range of 0.5 inches to 5.0 inches, more preferably 0.5-2.5 inches and over a length in the range of 0.5 inches to 10 inches or more with a ratio of uniformity of dispensing from edge regions to a central region within a range of 0.5 to 2.0 or more preferably within a range of 0.67 to 1.33 or even more preferably within a range of 0.9 to 1.1.

In some embodiments, the needles are configured to extend perpendicular to a local surface of the a carrier body (e.g. radial from a cylindrical body) though in some embodiments the needles may extend with from the body so that a tangential component also exists (e.g. to make the tips of the needles more perpendicular to a surface of the skin at the instant of initial penetration or at the instant of final extraction.

The cylindrical body may include at least one region around its perimeter where relative orientation of the handle and the cylindrical body are such that no dispensing of a drug occurs and/or such that needles are not extended from the cylindrical body.

Pluralities of needles may formed as part of insertable cartridges (e.g. linear arrays, small sets of linear arrays or the like (e.g. 2-4 rows of needles). The device may include receptacle for retaining the replaceable cartridges, flow paths between a replenishable reservoir and a refill vial or refill needle. In some alternative embodiments, drugs may be provided within the needle cartridges themselves on a initial fill basis or on a refillable basis.

In some embodiments, needles may include one or more distal apertures. For example, in some embodiments, the needles may include two apertures that dispense material in opposite directions, three apertures that dispense material at 120 degree angles, or four apertures that dispense material at 90 degree angles. In some embodiments, neighboring needles may be displaced along X and Y directions so as to yield more uniform distribution of dispensed material. In some embodiments, dispensing or jetting locations of neighboring needles may be differently orientated to yield more uniform dispensing of material. In some embodiments, needle head may jet material radially from the longitudinal axis of the individual needles, with a tangential component along the circumference of the individual needles (e.g. 1-90 degrees), while in other embodiments jetting direction may be include a tangential component (e.g. 1-90 degrees) along the longitudinal axis of the needles, while in still other embodiments, jetting directions may be have components in each of the tangential, radial, and longitudinal directions. In embodiments where jetting direction has a tangential component, it may be desirable to have needle heads (distal ending including orifices) mounted rotatably on needle bodies so that jetting direction can vary based on jetting velocity, and the like. In some embodiments, as opposed to using side ports on the needles to dispense or extract material, some or all of the needles may include end ports which may advantageously or disadvantageously result in coring of the tissue during insertion.

The various device embodiments of the invention provide improved intradermal and/or transdermal microneedles or arrays of such microneedles. In some of these embodiments the needles are generally retracted and are made to extend beyond a tissue contact surface of the device only during operational use (e.g. delivery of material, extraction of material, or for signal reception or signal delivery). In some embodiments needles may be located at a relatively uniform spacing around the periphery of a rotating tissue contact element (e.g. a cylinder) while in other embodiments needle placement may be limited to only certain portions of cylinder or slider (e.g. a linear array or closely spaced set of parallel linear arrays).

In some preferred embodiments, control of fluid flow into or out of the needles selected needles is desirable. In particular, in some embodiments an uncontrolled amount of medication may be delivered if delivery can occur from a plurality of needles regardless of whether those needles are actually inserted into tissue or not, let alone whether their delivery port or ports are at an appropriate depth into the tissue.

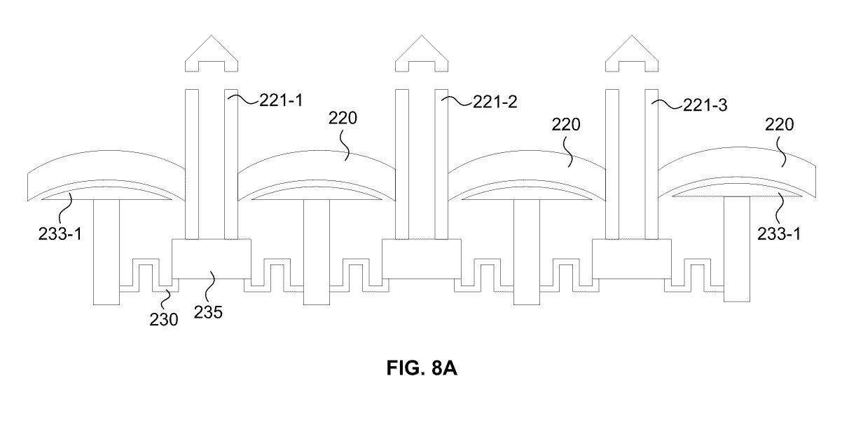

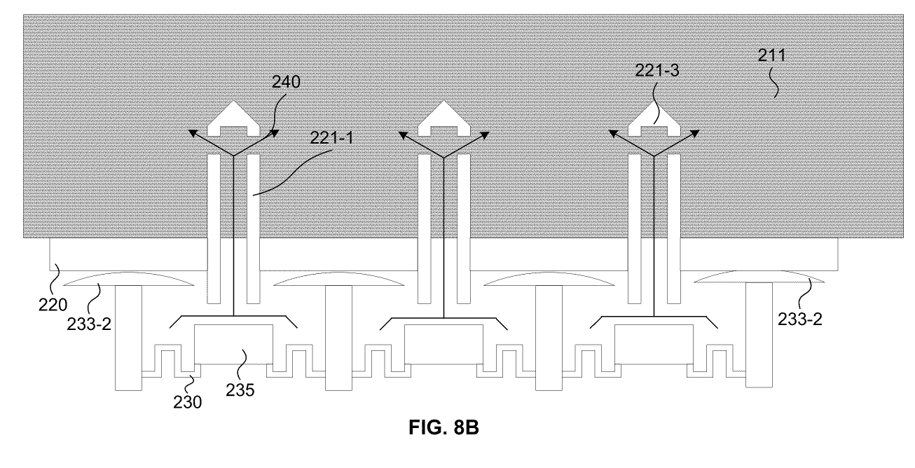

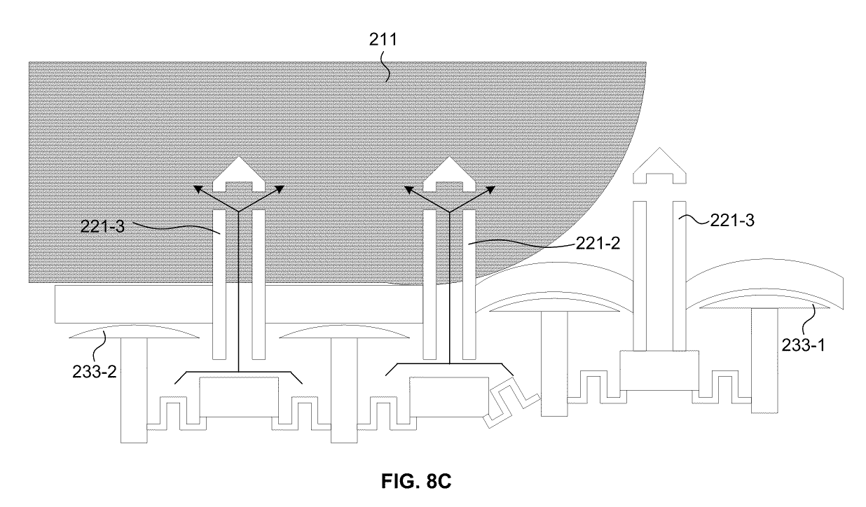

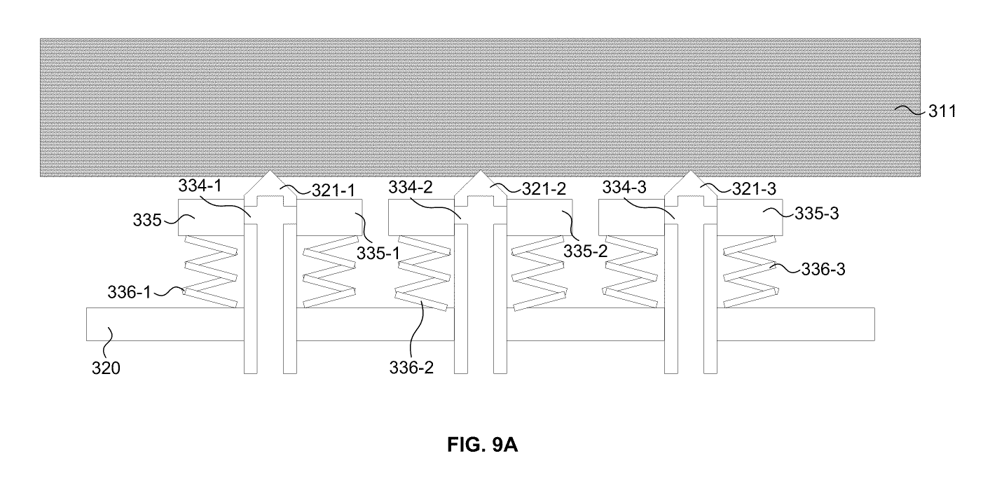

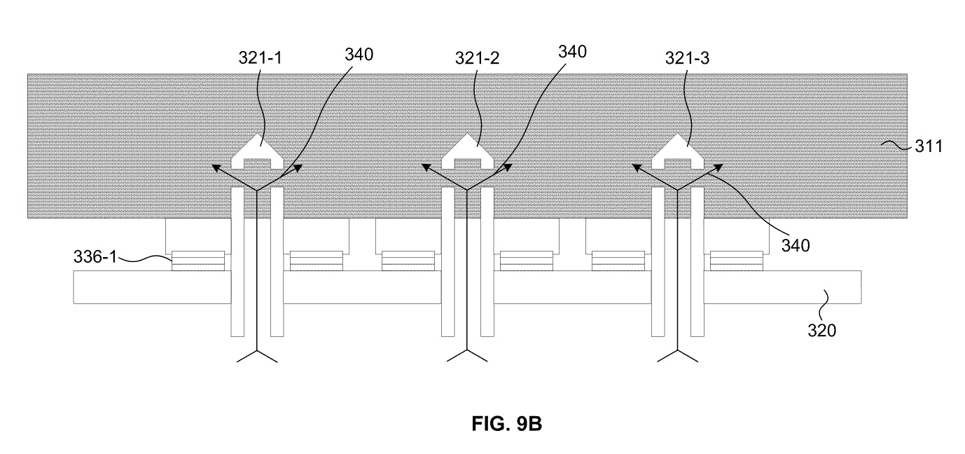

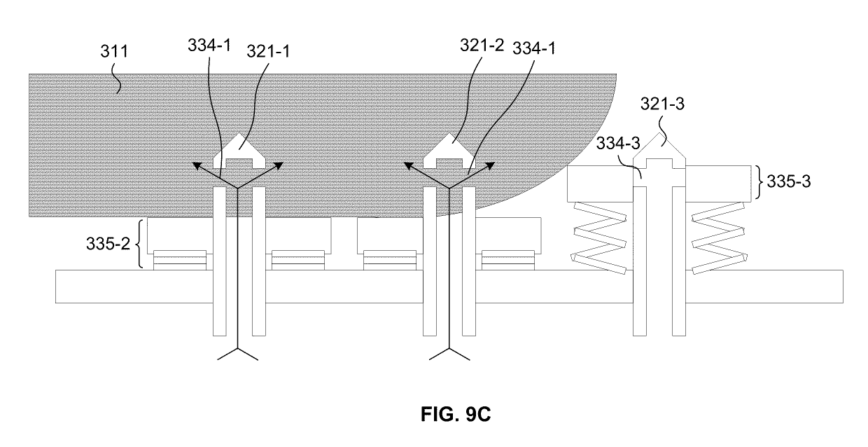

FIGS. 7A-7D provide various methods for limiting unintended dispensing or extraction of material. However, additional methods and device configurations may be used to provide even further control of dispensing or extraction of material as will be discussed below in conjunction with FIGS. 8A-8C and 9A-9C and their variations. In some of these further alternatives, individual needles or groups of needles may be provided with valves or other flow-inhibiting structures which can be triggered on a needle-by-needle basis as the needles penetrate the target surface.

FIGS. 8A-8C provide various schematic views of a needle array according to an embodiment of the invention wherein individual needles include valve elements that inhibit flow of fluid from the needles if the needles are not pressed into or against a surface. Such needles may be useful in the various embodiments discussed herein above as well as those discussed herein after however particular useful may exist in embodiments like those of FIGS. 7A-7B where a barrier membrane does not exist to inhibit inadvertent administration of material.

FIG. 8A provides a schematic cross-sectional view of a plurality of needles 221-1 to 221-3 which are held by a membrane 220 and possibly by other structures (not shown). As can be seen the proximal ends of the needles abut valve seats 235 which are attached to spring elements 230 which are in turn attached to release mechanisms 233-1. Release mechanisms 233-1 may be held in a “closed valve” state by springs and/or other structural elements (not shown). As can also be seen each valve seat 235 is attached to adjacent release mechanisms on either side of each needle. In some alternative embodiments each valve seat 235 may be connected to a single release mechanism. In FIG. 8A as valve seats 235 are closed no flow or minimal flow can occur through needles 221-1 to 221-3. FIG. 8B schematically depicts the state of the process after needle array 221-1 to 221-3 has been pressed into tissue 211. Upon such pressing, membrane or surface 220 is compressed backward toward the proximal end of the needles thus pushing down release mechanisms 233 and driving valve seats 235 away from the proximal ends of the needles thus allowing fluid to flow from the needles into plaque 111′ via valves 240. In some alternative embodiments valves may be opened and closed multiple times and different fluids dispensed. In still other alternative embodiments fluid flow may be reversed such that material is drawn into the needles as opposed to being dispensed from the needles. FIG. 8C depicts an alternative state of the process wherein tissue 211 exists such that only a portion of the needles are inserted into the deposit and thus only a portion of the release mechanisms are compressed and thus only a portion of the needle valves are opened. In such cases the primary flow paths for fluid will be from only those needles which have had their valves opened.

FIGS. 9A-9C provide schematic illustrations of various states of a needle array configuration according to another embodiment of the invention where the needles include alternative valve elements which inhibit the flow of fluid from the needles when the needles are not pressed into or against a delivery or target surface.

FIG. 9A depicts needles 321-1 to 321-3 located in proximity to a target tissue region but not entering the tissue. The devices of this alternative embodiment include flow stops or flow seals 335-1 to 335-3 which are located beside needle outlets 334-1 to 334-3 unless they are forced backward toward membrane or surface 320 via compression of springs 336-1 to 336-3. While seals 335 are located above outlets 334 little or no fluid flows from the needles. In FIG. 9B the state of the process is shown where surface 320 has been expanded to drive needles 321-1 to 321-3 into tissue 311 which caused springs 336 to compress forcing seals 335 away from outlets 334 thereby allowing fluid to flow from needles 321 along paths 340. FIG. 9C like FIG. 8C shows a state of the process where only a portion of the needles encounter tissue 311 and thus only a portion of the seals are displaced to allow outlets 334-1 and 334-2 to open while leaving 334-3 closed. In such situations only the needles 321-1 and 321-2 will participate in fluid exchange.

Numerous alternatives and variations to the needle configurations and valve structures of FIGS. 8A-8C and 9A-9C are possible and they will be apparent to those of skill in the art upon review of the teachings herein. In some alternative embodiments some of the needles may be connected to a different manifold from that used by other needles such that a portion of the needles may provide one fluid or even fluid intake while another portion of the needles may provide a different fluid. In some such embodiments, some individual needles may include multiple ports and multiple lumens.

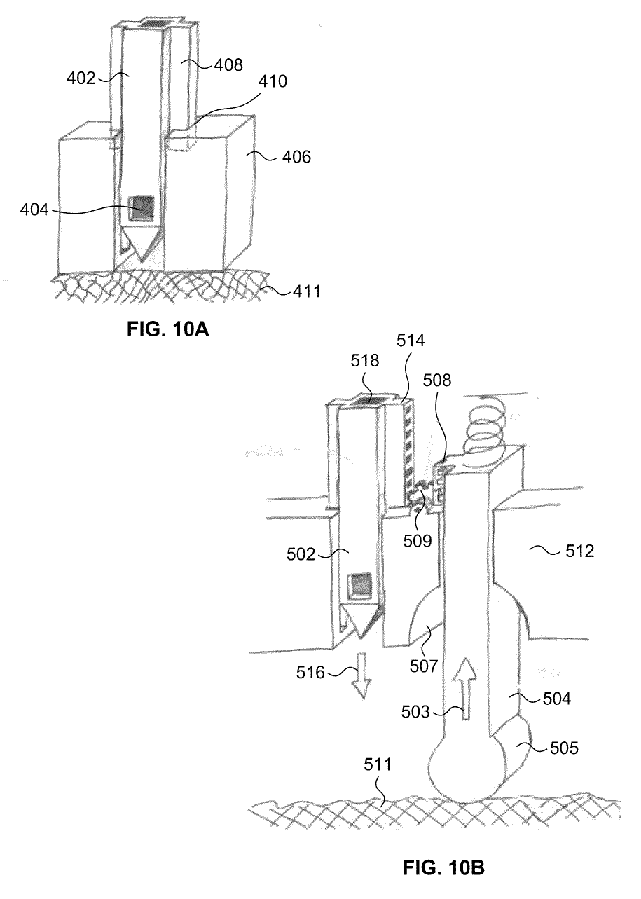

FIGS. 10A-10B provide two additional illustrations of retractable needle embodiments. FIG. 10A illustrate a retractable needle 402 wherein the needle includes a side port opening 404 and can move up and down along a slide bracket 406 which forms part of a needle housing via tracks 408 and guides 410. Once the needle is driven below the base of the needle housing it can begin penetrating into tissue 411. FIG. 10B provides another an alternative mechanism for driving a needle 502 into a tissue surface 511 by depression of a spring loaded arm 504 having an base of expanded cross-sectional shape (e.g. so that it does not damage the tissue) that contacts the tissue 511 and can be driven upward 503 into a notch 507 within a body of the needle housing 512 (e.g. a needle array housing for which one element is shown). As the push arm 504 is depressed toward the housing body a linear rack 508 on the push arm 504 engages a gear 510 which in turn engages a linear rack 514 on needle 502 driving it downward 516 from the body of the needle housing 512 and into the tissue.

Various alternatives to the embodiments of FIGS. 10A-10B are possible and include embodiments where (1) the arm and needle are connected via a gear configuration that gives enhanced needle displacement relative to arm displacement; (2) embodiments where the side port of the needle is seated against a side wall of the housing such that limited or no dispensing from the needle can occur prior to the needle port being driven below the bottom of housing, (3) embodiments where the push arm surrounds the needle or at least abuts the exit port of the needle such that dispensing from the need cannot occur until the push arm is moved past the outlet port; (4) embodiments where the inlet port 518, for the needle is blocked by an element (e.g. the inlet port could be on the side of the needle and it could be blocked by a sidewall of the housing, until adequate extension of the needle has occurred so as to cause the inlet port to align with a fluid feed path. Numerous additional variations are possible and will be apparent to those of skill in the art.

Fabrication Techniques:

In preferred embodiments the needles are formed at least in part from a multi-material, multi-layer fabrication process such as one of those described herein. Individually formed needles, partial needle arrays formed together, or even complete needle arrays formed together may be assembled with other device components after release of sacrificial material and before or after release from a fabrication substrate. In some preferred embodiments the needles along with associated valve and biasing structures may be formed in whole or apart using multi-material multilayer electrochemical fabrication methods. As part of the electrochemical fabrication process or separate from that process appropriate conformable elements (e.g. for use as valve seats or membranes may be formed or bonded to the needles).

Furthermore, the needles or needle arrays can be attached to or otherwise combined with other device elements to build up overall devices similar to those of FIGS. 5A-5C and 6A-6D or there variations and alternatives.

Treatment and Diagnostic Applications:

As noted above the devices of the various device embodiments of the invention may be used in a variety of methods to treat a variety of conditions. Some methods may involve the repeated steps of (1) sliding, (2) stopping, (3) needle deployment, (4) injecting or extracting material, and (5) needle extraction. Other methods may involve the repeated steps of (1) sliding, (2) needle deployment, (3) relative quick fluid injection or extraction without stopping, and (4) needle withdrawal. In still other embodiments, continuous rolling of a cylindrical element may be used and the process include repeated (1) needle deployment, (2) injecting or extracting material, and (3) needle extraction while continuing a relatively uniform rate of motion. Various other embodiments are possible and may be based on any combination of the above noted steps. Alternative embodiments may also combine steps from one embodiment with those of another embodiment to obtained enhanced treatment or diagnosis.

In various embodiments, the timing of needle deployment and extraction may be user controlled, controlled via automatic movement detection, or controlled via operator response to signals generated by the device (e.g. lights or sounds) in response to automatic movement detection. Use may involve an initial shaving, cleaning or other preparation of target area as well as post treatment operations (e.g. bandaging, application of antiseptic, or the like). In the case of reusable devices, device sterilization may occur between uses and or needle and/or drug replacement or replenishment may occur between uses (e.g. via hypodermic refill or replacement of drug or needle packets).

In some embodiments, drugs may be dispensed while in other embodiments, high pressure water or saline may be dispensed, while in other embodiments, the needles may be used to extract materials (e.g. intercellular material or even blood) from the body of the patient. For example, water or saline jetting may occur at or slightly above the collagen level to effect a separation of the skin from the collagen (e.g. to destroy the collagen network).

In some embodiments, instead of using needles to supply fluids or extract fluids, the needles may be used as light guides for laser or other radiation wherein the distal apertures may be configured (with mirror-like surfaces) to direct the radiation out of one or more apertures to provide laser radiation treatment at numerous locations simultaneously or in any desired pattern. The mirror-like surfaces may be used to re-direct the radiation along any desired path (e.g. to provide bidirectional transmission of radiation). A sensor within the body of the patient or external to the body of the patient may be used to read the radiation to provide desired diagnostic information. In still other embodiments, all or a portion of the needles may be used as electrodes and even individual needles, or groups of needles, may be formed with dielectrically isolated portions so that different signals may be sent to received by these different portions.

In some embodiments, devices may be sold with drugs or with the capability of being filled with drugs by end users or the like.

In addition to treatment applications (preventative and therapeutic) and diagnostic applications mentioned elsewhere herein, microneedles and microneedle arrays of various embodiments of the present invention may be used in a variety of applications including (1) antibiotic delivery, (2) antipruritic agent delivery, (3) anti-inflammatory agent delivery, (4) analgesic delivery; (5) treatment of localized infections such as abscesses where more targeted and smaller dosages can lead to improved outcomes with less systemic effects; (6) delivery of drugs to treat and preferably destroy lesions such as moles; (6) treatment of other skin conditions; (7) delivery of drugs to treat other conditions where oral delivery through intestinal track is not possible or relatively ineffective; (8) testing and/or treatment of allergies, (8) delivery of large proteins, (9) delivery of nucleic acids, (10) delivery of vaccines, (11) delivery of fragile antibodies, (12) delivery of hormones, (13) delivery of small molecules; (14) delivery of drugs with low oral bioavailability, (15) delivery of drugs with narrow therapeutic range, or severe side-effects, (16) delivery of parenteral drugs with short half-lives, (17) delivery of injectable drugs having a high incidence of site reaction. In addition to material delivery, the needles may also be used in general for sampling purposes such as (1) biopsy/tissue acquisition; (2) aspiration, and/or (3) in-vivo sensing of fluid (e.g., interstitial fluid, which can be analyzed for such constituents as glucose, hormones, blood gases, and therapeutic drugs). Also, the needles may be used to restrict tissue motion temporarily or permanently (e.g., grasping, securing, or stabilizing tissue) and for affixing other devices. Furthermore, needles may also be used for monitoring electrical conditions at the surface of the skin (e.g. monitoring electrical signals associated with muscle activity) and/or applying desired electric signals to the body for therapeutic, diagnostic, or preventative care with or without delivery of drugs or other materials or extraction of materials from the body.

In some embodiments the devices and methods may be used for hyperhydrosis applications (e.g. involving the injection of Botox or other materials) to reduce perspiration in selected body areas (e.g. hands, arm pits, etc.).

In some embodiments, microneedles—and particularly, dense arrays of microneedles—may be used to relieve localized edema by aspirating excess interstitial fluid, in lieu of, or as an alternative to drugs such as diuretics and vasodilators or the use of ultrafiltration.

In some embodiments, microneedles may be used to deliver agents such as depilatory chemicals (or agents toxic to the hair follicles, to the roots of unwanted hairs for more effective and permanent removal hair removal than is normally possible using chemical depilatories applied to the skin surface.

In some embodiments, microneedle arrays may be used to remove tattoos by delivering suitable agents such as those which can bleach the dyes injected to create a tattoo. The depth of delivery of the agents should be close to the depth of the dye within the skin (e.g., 50-1000 μm). Since the radius of bleaching for each microneedle is determined by diffusion of the agent (though in some embodiments the agent can be delivered under pressure), preferably the density of needles in the array is high. If the density is not sufficiently high, the array may be withdrawn from the skin, indexed by a distance less than the inter-needle pitch, and re-inserted to deliver additional agent.

In some embodiments, hair regrowth may be stimulated by directly delivering growth agents such as minoxidol to hair follicles or the region surrounding them, using arrays of microneedles. Again, if the density of microneedles is not sufficiently high, the microneedle array may be withdrawn from the skin, indexed by a distance less than the inter-needle pitch, and re-inserted to deliver additional growth agent.

In still other embodiments the needles or needle arrays may be used for the controlled delivery of insulin, vaccines, growth hormones, anti-osteoporosis drugs, antibiotics, analgesics, chemotherapy agents, anesthetics (e.g., for numbing of oral tissues to allow dental procedures), thrombolytic agents, drugs for blood pressure regulation, drugs for addiction therapy, microorganisms, gas bubbles, cosmetics, perfumes, depilatory and hair growth-enhancing agents, moisturizers, sunscreens, and therapeutic or diagnostic nanoparticles and microparticles. The micro-needle devices may also be used to perform allergy testing, including parallel delivery of multiple test agents using a single multi-needle array.

Electronic implants such as RFID devices or neuro or muscular stimulation devices, both tethered and self-contained, may be implanted by the micro-needle devices. Electrodes, both completely internal and communicating with an external device, may be implanted (e.g., in parallel) using the micro-needle devices. Lesions such as tumors, as well as skin defects and blemishes, may be treated by direct delivery of therapeutic agents into the lesion; these agents may include metal particles which are heated (e.g., by magnetic fields) in order to ablate unwanted tissue or nanoparticles such as folate coated buckyballs, carbon nanotubes, or other surface treated particles which may be selectively absorbed by selected tissues and thereafter heated by application and selective absorption of near infrared radiation, other electromagnetic radiation, or the like, to cause death of selected tissues (e.g. cancerous tumors). Skin markings such as tattoos and animal tracking numbers may be formed using the micro-needle devices to deliver colorants such as dyes into the dermis or other skin layers; if delivered relatively superficially, these colorants may be more readily removed or may have a shorter lifetime than conventionally-delivered skin colorants.

The micro-needle devices may be used to improve the health and youthfulness of skin through stimulation of a healing response. Improved coupling or the elimination of an external coupling fluid when delivering ultrasonic energy to the body for diagnostic, preventive, or therapeutic (e.g., high-intensity focused ultrasound) purposes may be achieved by coupling the energy directly to underlying layers of the skin and/or interstitial fluid through the micro-needle devices. Optical fibers for diagnostic or therapeutic purposes may also be inserted into the skin using the micro-needle devices as a conduit. Also, the micro-needle devices, may serve as conduits for body fluids, e.g., a small-scale bypass graft.

The micro-needle devices may interface to the human body through a rigid patch (e.g. planar or convex) or flexible patch (e.g. bendable and/or stretchable along the surface of the patch) applied to the skin, which in some embodiments is self-contained and in some embodiments contains a reservoir of the agent or drug to be delivered. The micro-needle devices may communicate with a pump, hypodermic syringe or equivalent, pressurized reservoir, or other device able to apply pressure to a fluid to be delivered. In some embodiments, capillary or electrocapillary forces may be used to retain and transport fluid within a needle; needles may be provided with internal sub-lumens or surface textures to enhance capillary effects, or internal electrodes and insulated regions to create electrocapillary effects.

### Microneedle Configuration Examples

The various device embodiments of the invention may make use of microneedles having a variety of configurations. Particular configurations may help needles penetrate target tissue, may help needles penetrate target tissue to controlled depths, may provide desired direction flow of dispensed material, may help provide more uniform distribution of dispensed material, or may help needle retention once inserted. Delivery through the needles may be into the dermal layer of the skin, or into other skin layers, or be subcutaneous or in some variations even intramuscular. In some embodiments the micro-needle devices are hollow (coring or non-coring), while in other embodiments some or all of the needles may solid (i.e., lacking an internal lumen).

### First Example

### Tapering Needle

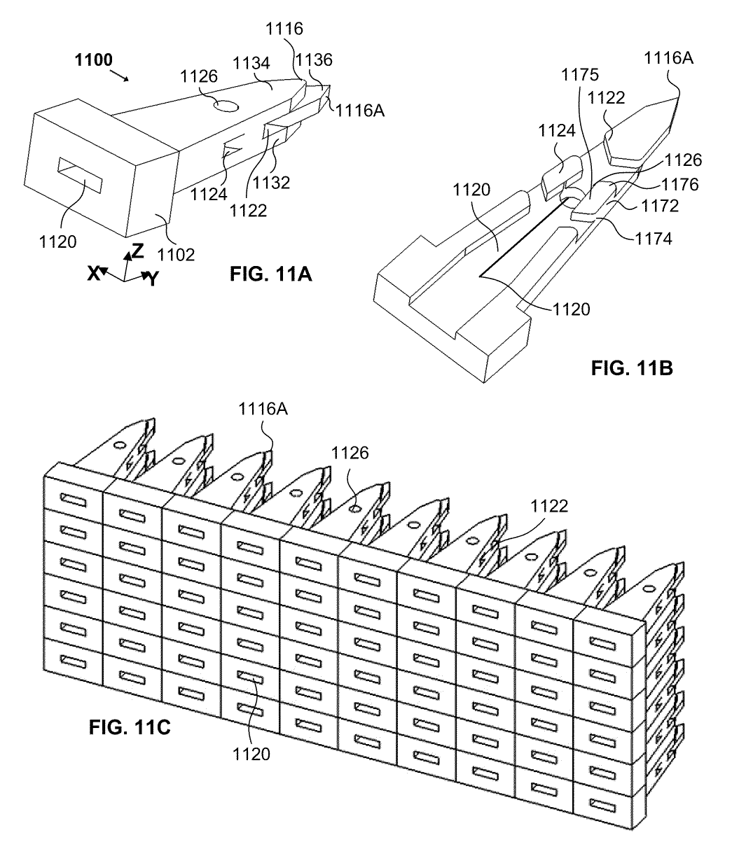

FIGS. 11A-11C depict, respectively, a perspective view of a needle 1100, a cut view of the needle of showing internal passages, and an array formed from such needles for use in some examples. In some implementations of the example and its variations, the needles are formed from a multilayer, multi-material electrochemical fabrication process. To minimize the number of layers that must be formed and/or to minimize the impact that discrete layer steps will have on the structure as formed, the needles may be fabricated using a specific orientation. In the present example, the needles are formed with the stacking axis of the layers being the Z-axis while the planes of the layers are X-Y planes. To reduce stacking height, the longitudinal axis (i.e. the longest axis of a single needle, i.e. the Y-axis in the present example) may be oriented in the plane of the layers and to further minimize the stacking height of layers, the thinnest transverse direction (i.e. the Z-axis in the present example) may be oriented along the stacking axis or alternatively, the structural elements intended to have the most smooth, curved, or sloped features (i.e. those defined by the X-Y plane in the present example) may be formed within individual layers. The needle configuration of the first example includes an inlet hole 1120 (assuming, in the present example that fluid flow is outward through apertures in the tip of the needle), and six outlet holes 1122, 1124, and 1126 of which three are visible in FIG. 11A. The two vertical holes 1126 (of which only one is visible in FIG. 5) have openings that extend along the stacking direction, and the four horizontal outlet holes (of which two are visible 1122 and 1124) have openings that extend in the plane of the layers. The lower halves of the four horizontal holes and the lower of the two vertical holes are visible in the cut or sectioned view of FIG. 11B. At the distal end of the needle is a tip line, or primary edge, 1116A which is preferably sharp to aid in penetrating the skin or other tissue to be crossed. Fluids pumped into the inlet hole are transported toward the outlet holes, from which they issue and enter the tissue. In some examples, fluids may be transported by capillary forces and/or diffusion from a reservoir through the outlet holes of the needles, avoiding need for pumping. In other examples, pumping may be provided by a mechanical pump, by a pressurized reservoir or bladder, electro-capillary forces, and other means. In the present example the needles include a base portion 1102 near the inlet having substantially parallel outer walls (which may be used to join or contact other needle elements when formed or located in an array) from which the needle extends in a progressively narrowing configuration (e.g. triangular configuration) from the base 1102 along the body 1104 to the tip region 1106. The needles of the first example include an internal passage. In some examples, to provide improved control of fluid, the internal passage may have a configuration, size, or features that cannot be readily fabricated by more traditional machining, molding, or casting approaches. A plurality of needles may be formed as a group or assembled so that the proximal ends of individual needles define a base or plate with the inlet holes forming perforations in that plate.

In the present example, the needle 1100, excluding the base 1102, is formed of three distinct cross-sectional regions which may be formed from three layers or from more than three layers (i.e. 2 or more layers for at least one distinction cross-sectional configuration. The three regions form a lower short prong 1132, a longer central prong 1136, and an upper short prong 1134 with the inlet hole 1120, and the horizontal openings 1122 and 1124 formed in the layer or layers defining the central prong while the vertical outlet holes 1126 are located in the layer or layers defining the lower and upper prongs. The distal end of the longer central prong ends in the primary penetration point, blade, or edge 1116A of the needle while the upper and lower short prongs end in secondary edges 11168 which may or may not have relatively sharp configurations.

As seen in FIG. 11A, horizontal openings 1122 and 1124 are located on the distally narrowing sidewalls of the central prong. As the proximal end 1172 of each aperture is displaced beyond (i.e. more radially distant) the more distal end 1174 of the respective aperture, relative to a central axis of the needle 1170, risk of tissue entry (i.e. coring) into these apertures could be exacerbated by the proximal ends having sharp edges with inward and distally facing slopes. On the other hand, as shown, tissue entry can be reduced by having the proximal end formed with rounded features and/or by having the more radially displaced portions including a proximally extending taper (beginning at edge peak) such that tissue contacting them while moving from the distal end toward the proximal end of the needle will tend to be forced outward and away from the interior of the openings. Such tapering may be configured to begin at positions closer to the central axis of the needle than outward edges of the upper portions 1174 of the respective opening(s). In some alternative configurations the entire surface forming the proximal opening may have an outward and distally facing slope.

In the present example the horizontal holes nearer the tip of the needle may be larger than the vertical and rear most horizontal holes to provide more balanced fluid delivery (e.g. by compensating for the increased fluid flow resistance that may exist in the progressively narrowing needles. In some examples, the holes sizes may be dictated by the desired fluid delivery depths. In still other examples, the holes on the opposite sides (either vertical or horizontal may be located at different positions along the length of the needle to provide a more optimal fluid delivery when delivery occurs via arrays of needles. In some examples, spacing between adjacent needles may be varied to provide more optimal delivery and/or needles may be provided with different lengths or aperture positions (e.g. needles in the central portion of the array may be longer than needles near perimeter of the array or alternatively apertures in the central needles may be located closer to the tips of the needles.

In some alternatives to this example, the needles may have square bases instead of rectangular bases or they may have bases of other desirable configurations. In other alternatives the needle may have bases with beveled edges or other edge features that allow only mating when orientations are properly aligned or that provide locking together of adjacent needles. Instead of the needles (excluding the base elements) being formed from three distinct cross-sectional configurations, the needles may be formed from more than three distinct configurations. For example, instead of having a lower short and narrower prong 1132 adjacent a longer central prong 1134 which is in turn adjacent an upper short and narrow prong 1132 (as in the first example) other configurations are possible. In some examples, different needles in the array may take on different configurations or different orientations relative to other needles. For example the X and Y configurations of some needles may be reversed so as to provide the array as a whole with a more uniform or isotropic set of mechanical properties). In some examples, the passages through different needles of the array may be connected (e.g. via manifolds) to different fluid sources so that a mixture of fluids may be applied to the body of a patient without premixing of the fluids or so that some needles may be used to deliver fluids while other are used to withdrawal fluids. In still other examples different numbers and locations of inlet and outlet holes may be provided. For example, as illustrated holes may be located along different surfaces, in different radial directions, in different longitudinal directions, and or with different angles relative to the longitudinal or radial directions.

In some example, the needles may carry a drug or other material on their outer surface and the outer surface may be textured with features designed into the layers of the device or via texturing that is thin relative to the layer thickness so as to provided improved storage or to an extended release profile for the drug or other material. Similar interior lumens or cavities with the needle may like-wise include texturing or designed passages for improving a release profile of the drug or other material. Such texturing may define porous matrices and the texturing may be made of a material that is biodegradable or

In some example portions of the base may include through passages not connected to a fluid path that extend through the base to the needles so that a vacuum or material may be pushed or pulled from one side of the base to the other side (e.g. a vacuum to draw tissue up to the front side of the base to help tissues in central region of the needle array to be brought into contact and penetrated by the needles. After penetration of the needles through the skin is achieved, the vacuum or suction may be released or removed and even replaced by a fluid. In some examples the secondary edges 11168 may be made sharp so as to avoid them acting as tissue mating impediments. Such sharp secondary tips may be formed by designing the needles as cross-bladed structures which upon 3-D to 2-D data conversion the up-facing and down-facing oriented blades form sharp tipped structures.

In some alternative examples, the apertures and possibly the needle surface may be coated over with a dissolvable material or with a porous material to provide a delay in drug delivery or an extended release profile. In other examples, only certain needles may receive such a coating such that one set of needles delivers the drug or other material during an initial period while the coating material is dissolving and thereafter the needles that had the coating may start to deliver the drug, or a different drug or material to provide even a further extension in the extended release profile. This staggering of delivery may be accomplished using two set of needles or more than two sets of needles (e.g. more than one coating material or different thicknesses of coating material.

In some examples, the needle tip or retention barbs, or the like may be made from a dissolvable material (e.g. the drug or other biodegradable material) such that when the needles have reached, or are nearing a removal time, the barbs or other retention features are eliminated or reduced so that only a fraction of their original retention force remains (e.g. less than 50%, less than 25% or more preferably less than 10% of their original retention or gripping strength).

### Second Example

### Needle with Hips

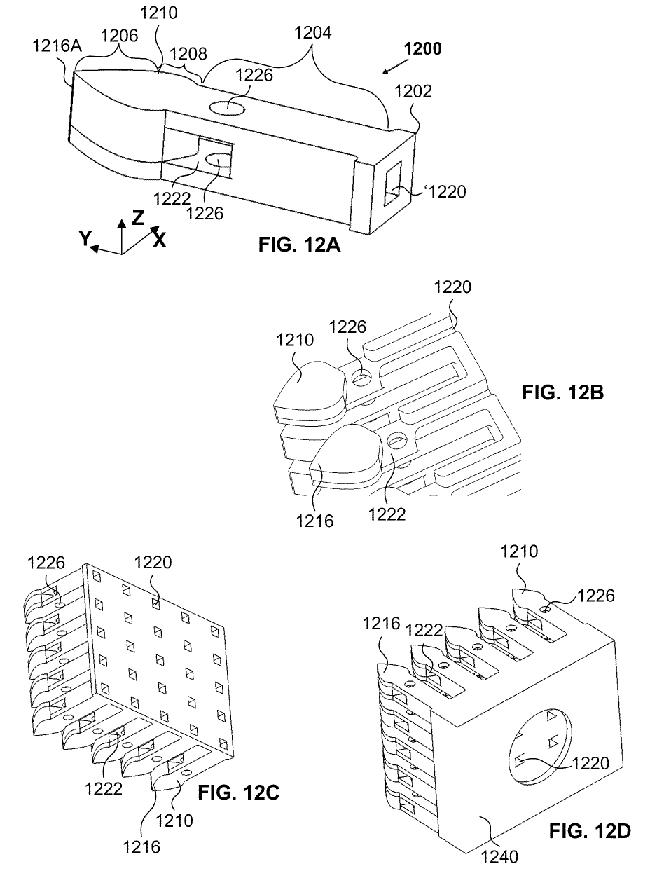

FIGS. 12A-12D depict a needle 1200 and an array of needles such needles according to a second example of needles that may be used in some embodiments of the invention. The needle 1200 of the example of FIGS. 12A-12D includes an inlet hole 1220 (assuming material is to flow from the proximal end of the needle to the more distal outlets), two vertical outlet holes 1226, and two horizontal outlet holes 1222, and is also sharp at its distal tip or edge 1216. The needle features a bulged hip 1210 that tapers down in region 1206 to edge 1216. The hip 1210 is also defined by a maximum lateral extension that extends from the radial extent of the shaft or body portion 1204 of the needle via region 1208. The regions 1208 and 1206 may be considered to define an “arrowhead” (i.e. a portion of the needle that s positioned between the proximal and distal ends of the needle and that has a width that is greater than the width of an immediately proximal portion of the needle) which is intended to help minimize entrance of tissue into the apertures 1226 of which may help anchor the needle within the tissue to prevent inadvertent withdrawal, lessening the burden of externally holding the needle/needle array in place on the skin surface during a prolonged session of drug delivery. In some alternative examples, the thickness of the arrowhead portion, along the stacking axis (i.e. Z-axis as illustrated) may be extended by inclusion of a lower layer and inclusion of an upper layer which could also provide additional retention force as well as shielding for apertures 1226. In still other alternatives the arrowhead at its distal end may take be shorter in the Z-axis by making the upper and lower most portions not extend all the way to edge 1216 but instead making them end more proximally (e.g. like the illustrated devices of the first example such that secondary edges exist.

FIGS. 12B-12C show the needle arranged into an array with proximal ends joined into a continuous plate. FIG. 12B shows a row of needles, in the array, in a sectional view so that internal passage of the needles may be seen.

FIG. 12D shows the needle array of FIGS. 12B-12C with the addition of a box-like manifold 1240 that delivers fluid to the array of needles In some variations of the second example, the “hipped” needle tips may take on different distal facing sloped configurations and proximal facing sloped configurations.

In the present example as well as in some other examples, the micro-needle devices include one or more elements to enhance retention in tissue. In FIGS. 12A-12D the distal end (i.e. arrowhead) of the needle includes a gradual arrowhead-like widening. Although more force may be required to push such a needle into tissue, it is more likely to be retained, particularly as the tissue flexes.

### Third Set of Examples

### Enhanced Tissue Gripping Tins

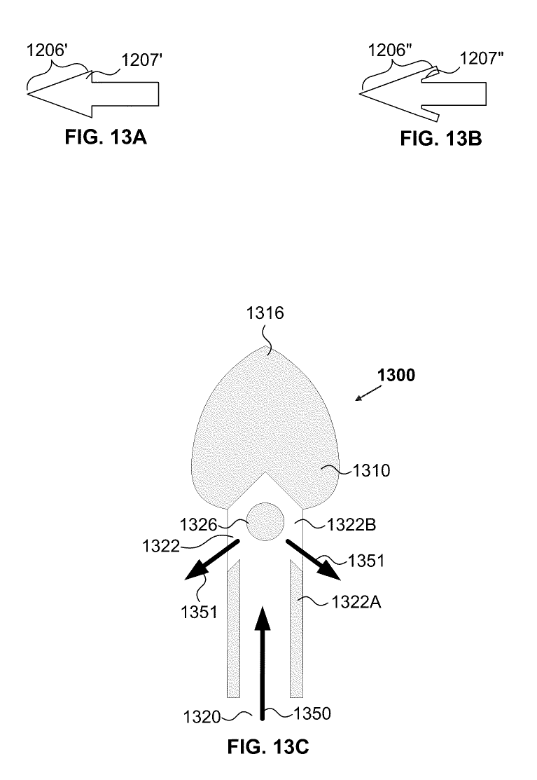

In lieu of arrowhead-like shapes, more barb-like structures may be provided such as those of FIGS. 13A and 13B. As seen in FIG. 13A the needle ends with a distal facing taper in region 206′ which ends proximally in a diameter reduction 1207′ that is drops radially from the maximum radial extent of the head to the radial dimensions of the shaft. In FIG. 13B, the distal end of the needle includes in a distal facing taper in region 206″ whose proximal end includes a reentrant feature 1207″ that can be used to hook tissue into which it is inserted. Needle heads, or distal ends, may provide even more secure retention, although some tissue damage may result when the micro-needles with such features are removed. In some variations of the examples the more radial portions of the retention elements (e.g. of FIGS. 13A and 13B) may be formed of a dissolvable material that is configured to at least partially dissolve by the time the needle or needle array is to be removed. In some examples, the needles are themselves not equipped with retention elements, but rather, the micro-needle device as a whole is so equipped. For example, a micro-needle array may include barbed elements attached to the base or manifold which serve only to secure the array to the tissue, and not for delivery or sampling. Such barbed elements may be in the form of solid (vs. hollow) needles with appropriate geometries, whose shapes, dimensions and locations may be similar or quite different from the micro-needles that are part of the same micro-needle device. In some alternative examples the added retention elements may be provided by dissolvable elements or other elements that tend to disappear as the needle array approaches its anticipated end of life so as to make removal easier.

In some examples, retention elements may be moving, vs. static. For example, once a micro-needle is inserted into tissue, anchoring elements may be extended from it to increase its retention by the tissue. These elements can then be retracted prior to withdrawal of the micro-needle from the tissue, if desired. In some examples, extension and/or retraction of the elements are achieved automatically by insertion and removal of the needle through an appropriate mechanism (e.g., linkages actuated through protruding elements on the micro-needle base which are depressed by skin contact). In other examples, a separate mechanism may be used to extend and retract the elements when desired. In lieu of separate anchoring elements, in some examples, the needle itself may change its shape (e.g., a split cylinder which opens like a cotter pin, or a needle which curves upon or after insertion) to provide enhanced retention in tissue or for other reasons. For example, needles may be made to curve, either upon or after insertion, according to several methods. In one such method, the needles are pre-curved, but held in a straight configuration by a relatively rigid wire that is inserted within their lumen(s) and then withdrawn before needle use, or by an outer sleeve that is retracted. In another method, the needles are provided with flexural joints which can deform in one direction but not as readily in other directions; tissue forces acting upon the needle as it is inserted can then cause it to adopt a new, curved shape. In general, by incorporating retention elements in the micro-needle devices, the need for external means of retention (e.g., adhesive in an applied micro-needle-based patch) is reduced or eliminated. In some alternative examples, an array of needles may be made to have improved engagement strength by distorting the orientation of one or more needles relative to one or more other needles by for example, flexing a connecting base element (e.g. to make it transition from an insertable convex, concave, or planar shape to a different shape) to cause inward or outward relative rotation of the needles and when it is time to remove the needles the base element may be returned to or toward its original configuration.

In some variations of this example, the micro-needle device base or manifold is perforated or porous so as to allow air to be drawn out of the space between base/manifold and tissue, retaining the device in place by suction or at least to minimum forces that may otherwise would inhibit full engagement of the array with the tissue.

In some other variations of this example, moving elements may be provided on needles which displace tissue or fluid to create a space or pocket with enhanced capacity to improve ability to receive a deliverable or payload. In some examples, these moving elements may also provide enhanced tissue retention.

The variations noted herein above with this example may apply to the other examples set forth herein while the variations noted in association with the other examples may be applied, mutatis mutandis, to this example or to the other examples herein. For example many of the variations discussed above in association with the first example may also be used to derive alternatives to this second example.

FIG. 13C shows a top view of a needle according to a an implementation of a third examples where the needle design is similar to that of FIGS. 12A-12D, which is also preferably fabricated horizontally, but with the horizontal outlet holes angled proximally (e.g. the openings point in outward and in the proximal direction) to further reduce the risk of tissue entering the lumen of the needle.

The various alternatives noted above with regard to the first example may also be combined into variations of the second and third examples. In still further variations, deflectors (e.g. fixed, movable, or deformable may partially or fully cover the outlets during insertion and may be moved to an open state by fluid pressure or mechanical manipulation after insertion. Such deflectors or other elements (e.g. spring loaded or other biased balls, plates, flaps, or the like) may be useful in minimizing coring of tissue or in minimizing the back flow of fluid or tissue back into the needle (e.g. they may function as one way valves). If the purpose of the deflectors is to act as one way values, the deflectors may be moved into the passages. In other alternative examples, passages within the electrochemical fabricated needles may be coated with various desired materials to improve biocompatibility, fluid compatibility, compatibility of mixtures of biological materials and fluids being administered.

To achieve sharper tips, it may be preferable to fabricate the devices by pattern-depositing sacrificial material, blanket depositing structural material, and then planarizing, as opposed to a process in which the structural material is pattern-deposited and the sacrificial material blanket-deposited.

Micro-needles, including alternative designs, may also be fabricated vertically. In this case, methods may be used to provide sharp tips such as methods used to provide sharp tips on vertically-fabricated semiconductor testing probes as described in various patent filings by the same assignee some of which are referenced herein elsewhere (see, e.g. U.S. patent application Ser. No. 11/178,145, filed Jul. 7, 2005, which is incorporated herein by reference). Other methods may be used to achieve sharper tips such as mechanical abrasion and electrochemical polishing.

As exemplified in the first through third examples (e.g. as depicted in FIGS. 11A-13C), in some examples, hollow micro-needle devices are provided with apertures to allow exchange of material from the interior to exterior. Such apertures may be substantially orthogonal to the outer surface of the micro-needle devices, or at an angle to these outer surfaces. For example, apertures which are angled so that their outer portions are closer than their inner portions to the tips of the micro-needle devices (“forward-pointing apertures”) may be useful to help project material introduced through the micro-needle devices to a point more distal than the tip (e.g. the horizontal apertures as seen in FIG. 12B), while apertures angled opposite to these (“rearward-pointing apertures”) may be useful to minimize the introduction, or ‘coring’ of tissue when the micro-needle device or devices are inserted into tissue (e.g. the horizontal apertures of FIG. 13C).

In further variations of the first through third examples, pumps may be formed along with the needle arrays or may be combined with them after formation. Such pumps may, for example, take the form of bellows, gear pumps, check valve devices. In still other alternative examples, a hose connector may be used in conjunction with the other examples and in further variations it may be made to extend from the side of the manifold as opposed to its back.

### Fourth Example

### Needles with Primary and Secondary Cutting Edges

In the fourth example of a needle implementation that may be used in some embodiments of the invention, each needle is provided with multiple cutting edges, some of which are staggered or recessed with respect to others. Such staggering may be applicable to micro-needles that are fabricated either horizontally or vertically. Cutting edges are obtained by designing individual layers with sharp points or by designing three-dimensional structures with thin blades which upon cross-sectioning produce layers with fine leading edge points (e.g. when the needle is fabricated horizontally, i.e., with the long axis of the needle parallel to the plane of the EFAB layers and with a tapering blade extending vertically through two or more layers). The needle of FIGS. 5-10 has a primary cutting edge that is sharp while secondary cutting edges are relatively rounded and dull.

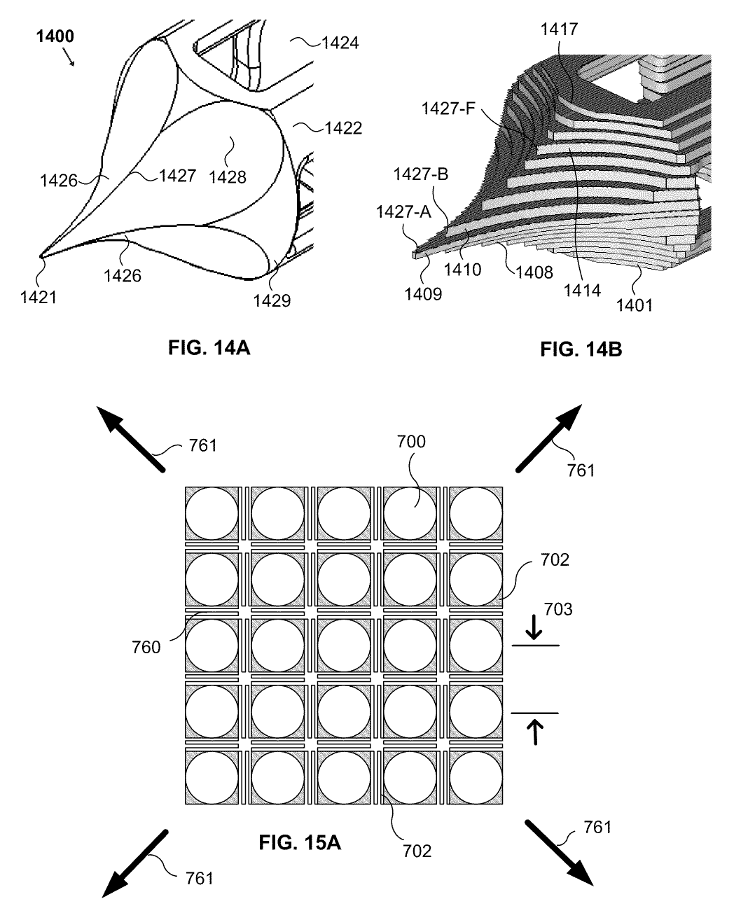

FIG. 14A provides a perspective of a smooth walled CAD design of a sample needle design of an fourth example implementation of a needle configuration that may be used in some examples wherein the configuration provides some features in common with the example of FIGS. 11A-11C but where the configuration provides secondary edges that are also sharp. The needle 1400 includes a tip 1421 formed by the conjuncture of a vertical doubled edged secondary blade 1427 and a horizontal double edged primary blade 1426. The needle 1400 also includes a plurality of outlets 424 that extent radially from a body 1422 of the needle. The edges 1427 and 1426 are joined at the tip and along primary distal tapering surfaces 1428 while adjacent primary surfaces 428 that are away from tip 1421 and blade edges 1427 are joined by secondary distal tapering surfaces 1429.

FIG. 14B provides a perspective view of the needle device of FIG. 14A after cross-sectioning the needle into 17 layers (1401-1417) where layer 1409 provides tip 1422 which is located at the conjunction of primary blade 1426 (defined by layer 1409) and a most distal portion of secondary blade 1427 which is labeled as 1427-A. As can be seen in addition to layer 409 providing a sharp primary cutting surface, elements 1427-B-1427-F associated with each of layers 1410-1414 and 1408-1404 respectively provide secondary (and even tertiary, quaternary, etc.) sharp cutting edges. These secondary or higher order cutting elements 1427B-1427F result from intersecting cross-sectioning planes with the sharp edge of secondary blade 1427 at a plurality of layer levels. It is believed that the needle designs of FIGS. 14A-14B provide needles with enhanced tissue penetrating capability. Of course, in other designs different numbers of sharp secondary features may be provided by varying the number of edges in the CAD design and/or by varying the number of layers that the needle is cross-sectioned into. Designs with sharp secondary edges may be formed directly from two-dimensionally designed needles (i.e. designs based on layer configurations and not on three-dimensional models). Needles have such sharp secondary cutting elements may also be formed from layers stacked along the longitudinal axis of the needle.

Due to the elastic nature of human skin, the tip of the needle has to be sufficient sharp to provide enough stress to cause initial penetration. For a no-bleeding and painless injection, it may be desirable for the effective length of the micro-needle to be only enough to penetrate the epidermis but not into dermis. As the needle length gets shorter however, the tip has to get sharper.

The needle design of FIGS. 11A-11C uses a sharp tip to penetrate the skin at the beginning of contact and then the rest of the needle penetrates the skin. As additional layers encounter the skin, larger resistance may be encountered as successive layers do not provide a sharp penetrating profile as their leading edges are rounded.

The needle shape of FIG. 14A provides sharp secondary edges 1427-A to 1427-F as well as a sharp primary edge 1422 or 1427-A. Instead of pushing into skin with the primary edge and then tearing and spreading with the secondary edges, the secondary edges also provided improved penetration capability (cutting). As the structure of FIG. 14A is cross-sectioned and formed using a multi-layer multi-material fabrication process as proposed herein, sharp secondary edges 1427-A to 1427-F will be provided even though the secondary edge is not continuous.

It is within the level of skill in the art in view of these teachings to experiment with or otherwise determine optimal needle length, layers thicknesses, numbers of layers, and edge angles to provide optimal penetration.

In some alternative examples to the first through fourth examples, micro-needle devices in an array of micro-needles may be varied in diameter, location/pitch, shape (e.g., cross-sectional shape, internal shape), or length within a single array. In some examples, needle geometry may be varied so as to achieve a rate of delivery or a sampling rate through the needle that varies with position in the array. For example, the tissue closer to the center of a needle array may have less capacity to accommodate a delivered fluid than tissue toward the edges of the array. Thus, it may be useful to tailor the flow rates so that outer needles in an array have higher flow than inner needles. In some examples, needles toward the center of an array are made longer and/or spaced at a larger pitch when compared with those closer to the edge of the array to achieve more even penetration of the tissue as the per-needle penetration force is greater on needles at the edge of an array since there are no neighboring needles over which to distribute the force. In some examples, needles which are initially longer for this purpose may retract or curve to a shorter configuration after penetration).

In still further alternatives to the first-fourth examples, the micro-needle devices may in include needles with shapes optimized for insertion and withdrawal from tissue, for delivery of a payload, and/or for sampling. For example, needles may be shaped (e.g., tapered along the distal-proximal axis) to minimize buckling during insertion. The needles may be non-circular in external and/or internal cross section (e.g., rectangular, triangular, finned, star-shaped). The needles may have a tailored, variable stiffness along their distal-proximal axis, and may also have anisotropic stiffness, such that they bend more easily along one axis than another. The needles may also have helical, fluted shapes much like drill bits.

In some alternative examples, the micro-needle devices include needles which bifurcate or branch off into more complex shapes (e.g., tree branch-like, sea urchin-like with multiple spines), for example, to increase the area available for payload delivery or sampling, or to achieve other effects. A similar effect may be produced by incorporating textures or projections on the needle surface. The needle may initially be simple and/or smooth, and then bifurcate, branch, form textures or extend projections after insertion.

In some alternative examples, the micro-needle devices include compliant elements. For example, compliant, bellows-like elements in the more proximal regions of a needle may allow bending to accommodate skin deformation. Also, micro-needles equipped with compliant elements may be used to allow a needle to seek its individual path of least resistance while penetrating tissue, potentially minimizing damage to structures such as blood vessels, nerves, and glands. In some examples, the micro-needle devices include needles which are able to change their lengths, for example, by incorporating flexural (e.g., bellows) elements, by incorporating multiple, telescoping segments, and so on.

The micro-needle devices may in still other alternative examples include needles having tips optimized for insertion and withdrawal from tissue, for delivery of a payload, and/or for sampling. In some examples, needle tips may be optimized for smooth cutting of tissue, while in other examples, they may be optimized for tearing tissue. In various examples, the tip may be in the shape of a cone, a flat blade (similar to a flat-blade screwdriver), a pyramid (e.g., with three or four sides), or a finned/star-like structure such as the tip of a Phillips screwdriver. In the latter case, the tip may have four sharp blade-like edges to ease penetration through tissue instead of relying only on a single distal sharp point as was described above with regard to the examples of FIGS. 14A-14B.

### Fifth Example

### Methods for Reducing Fabrication Costs and Improving Reliability of Fabrication

Various methods for reducing fabrication cost and improving reliability of fabrication are possible. A number of examples of such techniques are set forth below. Fabricating the micro-needle devices can be a challenge, both in terms of economical fabrication using EFAB technology and achieving sharp penetrating needle tips. With respect to the first of these, if the micro-needle devices require needles that are spaced widely apart (i.e., a large base/manifold), monolithic (minimal assembly), leak-free manufacturing using EFAB may be costly, since much of the build volume is empty. With respect to the second of these, it can be difficult to provide very sharp tips as-fabricated (i.e., without post layer formation processing), particularly if the needles are fabricated with their distal-proximal axes (i.e. longitudinal axis) perpendicular to the layer plane (i.e., “vertical fabrication”).

With respect to micro-needle materials, it is normally desirable that the materials should be resistance to fracture. High ductility materials and/or materials that are compressively stressed can minimize the risks of cracks and crack propagation.

In some examples, manufacturing cost can be reduced by building multiple needles with a higher packing density than desired in the final micro-needle array device. In some examples, needles are fabricated singly or in small groups, either vertically, horizontally (distal-proximal axis, or longitudinal axis, parallel to the layers), or perhaps in some other orientation. Needles/needle groups are then attached one at a time to a base/manifold, which may itself be produced by EFAB, but which would typically be made using a smaller number of layers (e.g., 1-5). It is of course preferred that there be no leaks between needles and the base/manifold.

In some examples, features on the needles/needle groups tightly engage features on the base/manifold (e.g., a press fit); adhesive, soldering, brazing, welding, and other methods may be used in conjunction with mechanical attachment of this kind to ensure minimal leakage. In other embodiments, no mechanical attachment per se is provided, and needles/needle groups are bonded using adhesive, soldering, brazing, welding (e.g., laser welding), thermosonic bonding, etc. To facilitate bonding by a biocompatible and payload-compatible solder, for example, one or both mating surfaces may be pre-coated with the solder (e.g., by electrodeposition) as part of the fabrication process for these parts, or coated afterwards. To allow for some misalignment between needles/needle groups and base/manifold without making the effective lumen vary between needles/needle groups, the aperture in the base/manifold can be different in size (e.g., larger) than the needle lumen; if the needle wall thickness is large enough to completely overlap the base/manifold aperture even if the needle is misaligned, then no leakage will occur after bonding. In some examples the base or manifold and/or needles themselves may include mating features (tubes, holes, and/or steps) that ensure proper alignment around which solder or other adhesive may be located. For example, solder may be located at the ledge of a step which includes a larger diameter elements and a smaller diameter element, such that the smaller diameter element (e.g. tube) may be fitted into a hole in another element to which bonding is to occur, and then the step and associated solder may be made to sit against a surface surrounding the hole and thereafter reflow and bonding can occur.

In some examples, bonding of needles/needle groups may be done using a pick and place robot in which each needle/needle group is picked up individually and moved to the correct location on the base/manifold, then bonded. In other examples, bonding may be done by supplying the needles/needle groups directly to the moving bonding head of a machine. For example, needles/needle groups may be fabricated with neighboring needles/needle groups temporarily joined by small tabs which can be broken off or stretched/deformed as each needle/needle group is bonded, or by a material (e.g., sacrificial material) which can later be removed by etching or melting. Needles may also be fed to a bonding head in tandem (end-to-end) through a feed tube, or if joined by tabs or removable material (such as sacrificial material) to a strip, may be fed directly to the bonding head.

In some examples, many needles may be bonded to the base/manifold in parallel, which can save time and cost in assembly by reducing the amount of time to individually align and bond needles.

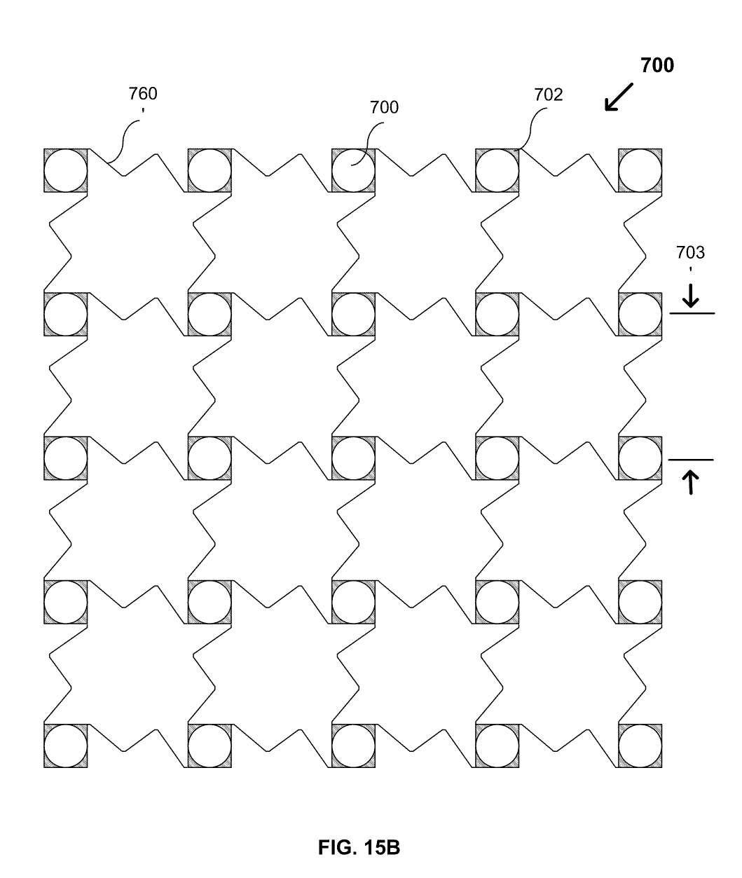

FIGS. 15A-15B provide schematic illustrations of an array of needles which are formed in a laterally compressed state (FIG. 15A) which may be connected by flexures and separated to a desired array spacing after formation (FIG. 15B) as an example of a method according to the fifth example such that higher lateral needle density can be achieved during fabrication with associated cost, time, and/or material consumption savings and such that group bonding may be more readily implemented.

FIG. 15A shows a plan view of an array of needles 700 (here 5×5) as-fabricated, with the inter-needle pitch 703 fairly small, so as to minimize wasted volume and to reduce cost and with adjacent needles joined by flexures 760. Each needle may optionally be provided with a separate base 702 at the proximal end of the needle, to which is attached flexures 760 capable of deforming plastically or elastically. In other variations, the flexures 760 may be directly attached to the needles, without a base. Prior to bonding, the array is expanded substantially uniformly into a new configuration (e.g., by pulling on the corner bases as indicated by the arrows 761 in FIG. 15A) as shown in FIG. 15B. In this configuration, the needles are substantially positioned correctly, and with a much enlarged inter-needle pitch 703′, for bonding to the base/manifold. Assuming the flexure deformation is not entirely plastic, the array may be maintained actively in the expanded configuration. Bonding may be performed in parallel (e.g., by placing the array and base/manifold in an oven to reflow solder) or individually (e.g., by laser welding). As before, by appropriate design of the base/manifold aperture and needle lumen size and wall thickness, some misalignment between needles and base/manifold may be tolerated. In some examples, a fixture or template may be provided to improve the alignment of the needles before or during bonding. In other examples, the expanded needle array can serve as a template to produce appropriately-located apertures in the base/manifold. To allow adjustment during bonding, in some examples bonding will start from a selected region of the array (e.g. a center region, a corner, or an edge) and work toward other regions such that applying or releasing tension from one of the corner (or other external portions of the array can result in movement of the needles to be bonded relative to the desired bonding locations such that proper positioning can occur without directly contacting needles that are being bonded.

In some alternative examples, the needles may be joined to a base manifold by locating their proximal ends on the base/manifold while in other examples, the needles may be slid into apertures in the base/manifold from their distal ends and then bonding made to occur while the apertures of the base our appropriately position along the needle shafts (e.g. near the proximal ends. In some examples the needles may be formed with extensions protruding below a base element where the extensions can enter the apertures in the base/manifold to a desired level (e.g. up to a stop formed on the needles) and then bonding made to occur.

Preferably the flexures are located near the proximal end of the needles only and so do not substantially interfere with the operation of the micro-needle devices. In some examples, the flexures may be removed subsequent to needle bonding (e.g., by laser cutting), while in other examples, they may serve as spacers between the base/manifold and the tissue (e.g., to minimize occlusion). The flexures may be formed from only a single layer of structural material or may be taller (e.g., multi-layer). In some examples, more than one tier of flexures may be provided at different locations along the distal-proximal axis. In some examples, a single multiple needle micro-needle array device may be fabricated using a single expanded array as described above. In other examples, more than one array may be needed to populate (e.g., by tiling) a single micro-needle array device, or a single array may contain a sufficient number of needles to populate multiple micro-needle array devices.

In some examples, in lieu of an array that expands in two directions as is described above, the array may expand only in a single direction. For example, the array may be a linear array (vs. a 2-D array as is described); such arrays are particularly useful when needles are fabricated horizontally. Another example is a 2-D array of needles in which the needles are produced at the final desired inter-needle spacing, or pitch, along one axis, but not along the other axis, and require pre-alignment expansion along that axis before bonding.

In some examples, in lieu of expanded flexures, bellows-like structures which can contain a fluid can be provided between needles in an array, allowing the expanded structure to serve as a manifold once expanded. This approach may be suitable for both single-axis and double axis expansion. In some examples in which 1-D expansion only is required (e.g. X-expansion), needles in each individual row or column of needles (e.g. in the y-direction) may be joined by a primary manifold and thereafter a secondary manifold may join the primary manifolds or the primary manifolds may be joined by a flexible or bellows manifold as noted above.

In some examples, in lieu of flexures, linkages with pivoting joints are provided. These joints may include built-in travel limits to prevent expansion of the array beyond a certain size. In some enhanced variations of this example, an actuation mechanism or mechanisms (such as one or more of the expansion devices disclosed in U.S. Provisional Application No. 61/018,283, filed Dec. 31, 2007 or in U.S. Non-Provisional patent application Ser. Nos. 12/134,188 (P-US210-A-MF) filed Jun. 5, 2008 and 12/179,573 (P-US225-A-MF) filed Jul. 24, 2008, each of which is incorporated herein by reference) may be used to spread or deploy the needles to their proper spacing along one direction or multiple directions.

In some examples, the flexures, links, or bellows that connect needles/needle groups are not the same everywhere, such that when expansion occurs, the needles end up in a non-uniformly spaced pattern that may be preferable for a particular micro-needle device, treatment or procedure.

### Sixth Example

### Needle Penetration Stops

Various examples may provide micro-needle designs and array designs which provide self-limiting of the depth at which the needles penetrate. In many procedures the stop may be set at an optimal depth at which micro-needles will penetrate tissue (e.g. the skin). The penetration depth should be deep enough to allow delivery of the drug, but shallow enough so that pain is held to a minimum.

An issue with some micro-needle configuration is that there is an optimal design for micro-needles which is dependent on tip sharpness, needle spacing and needle length. If a needle is not sharp enough or if too many needles are present, then the length of the needle must be long enough to provide enough pressure to penetrate the skin but once penetration occurs it may continue behind an optimal depth and thus reach a depth at which pain will occur.

In the examples to follow needle designs are presented where the depth of the needle penetration can be decoupled from the total length of the needle, which may make optimizing the needle design easier. Higher pressure can be achieved without the risk of penetrating too deep to cause pain. The needle may not need to be as sharp because the penetration force can be increased without penetrating too deeply. Penetration limiting structures are provided that limit the needles ability to penetration beyond a desired depth. Examples of features that provide such penetration limitations are set forth hereafter but it will be understood by those of skill in the art that many variations on the presented exemplary configurations will be possible. Limiting structures can be wider portions of needles, of groups of needles, independent structures may have larger diameters than the needle bodies, they can be platforms or a grid of bridging elements that extend between a plurality of needles.

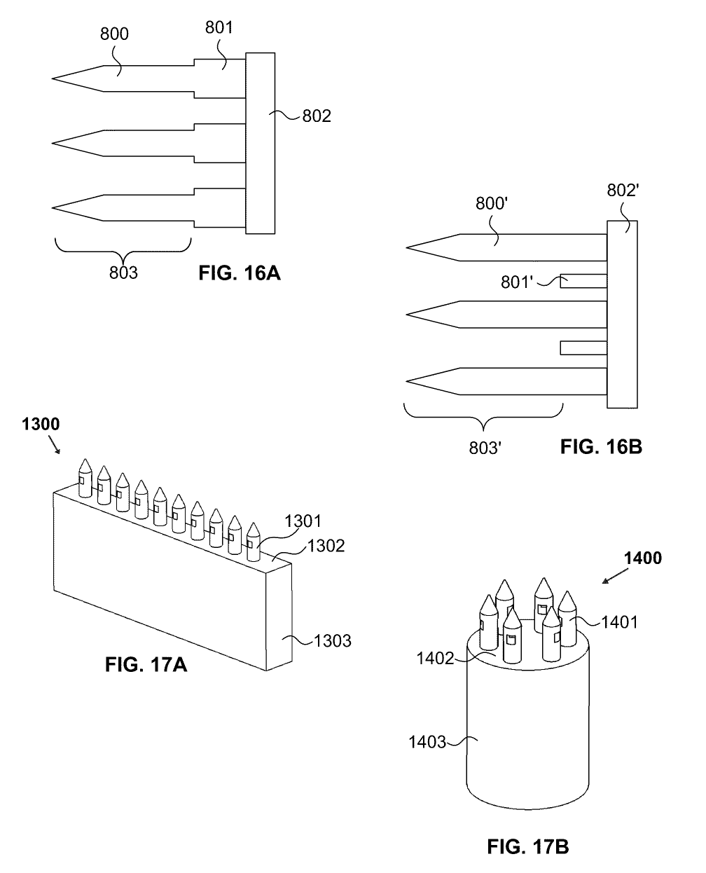

According to an implementation of the sixth example, elements may be added to control the depth of needle penetration into tissue. A first example of such elements are shown in FIG. 16A wherein the one or more needles 800 (all three as illustrated n the example of FIG. 16A) are joined to one another by a base 802 and include widened features 801 at a desired depth 803 below the needle tips. These widened areas resist further penetration of the needle or needle array into the skin or other tissue. FIG. 16B illustrates a second example according to the twelfth embodiment where stops 801′ are included that are independent of the needles but instead are attached to a base 802′ that also supports needles 800′. Such elements may be useful to avoid occluding the surface of a tissue which might otherwise occur by direct contact with a large base or manifold (e.g., for a micro-needle array); occlusion (i.e., reduced air contact) of the skin may result in irritation when it persists for an extended period. If the base does not contact the skin, then the skin is free to ‘breathe’ through the spaces between the needles. In some embodiments, the base or manifold is in direct contact with the skin, but includes channels, pores, or apertures through which the skin can breathe or through which fluid exchange can occur to the skin surface (e.g., to deliver an agent that mitigates skin irritation). Bases/manifolds may be formed of metals, polymers, ceramics, or other materials.

FIG. 17A provides a perspective view of a linear needle array 1300 and a two-dimensional array formed from a plurality of such linear arrays wherein each individual linear array includes a plurality of needles 1301, and stop feature in the form of a distal surface 1302 of a manifold 1303 for limiting penetration depth of the needles according to a second implementation of the sixth needle probe example.

Linear array of needles 1300 as shown on top of a box-like structure (e.g. manifold) 1303 effectively increases the length of the needles, therefore increasing the penetration force. The needle height (from tip to the stop feature 1302 on the manifold) is the optimal height at which the drug needs to be delivered. The needles cannot readily penetrate past the top surface (i.e. stop feature) of the box-like structure.

In alternative examples, the needles 1301 could remain connected in a linear array but the needles may be formed with extended proximal ends or stops that may be inserted between some of the needles as in variations of some earlier examples. In still other alternative the stops may join two or more needles together but less than all of the needles that form the individual array.

FIG. 17B provides a perspective view of a circular needle array 1400 and a multi-element array of such circular arrays wherein each individual circular array includes a plurality of needles 1401, stop feature 1402 defined by a distal end of a manifold or other base element 1403 such that for limiting penetration depth of the needles according to a third implementation of the sixth example. As shown, the individual needle arrays form circular groups of needles attached to a single manifold or base element whose top surfaces define a stop feature. Similar alternatives, as noted above in association with the second implementation are also possible for this third implementation.

### A Seventh Example

### Needle Arrays with Edge Penetration and a Side Port Outlet

As noted above numerous needle configurations are possible and readily formable using the multi-layer multi-material electrochemical fabrication process taught herein. Some additional embodiments of such variations of needle configurations are provided herein next where the tip of the needle elements is shifted from a center line of the needles to an edge or corner of the needles. Such shifting of penetration point can provide various advantages such as large outlets, outlets closer to the initial penetration point, or the like.

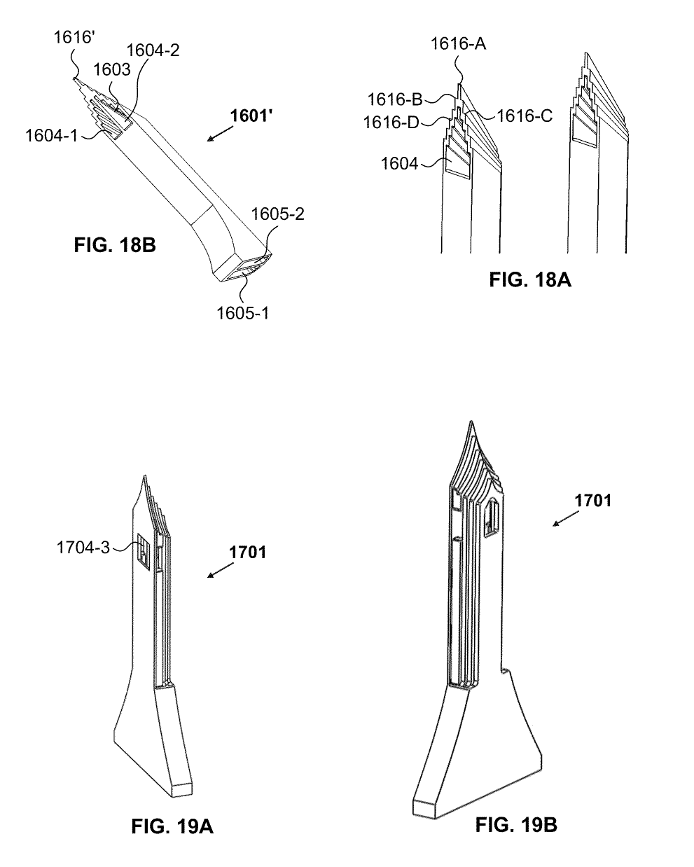

FIG. 18A provides a perspective view of an example linear array 1600 of needles 1601 supported by a base 1602 wherein the needles are designed for initial tissue penetration along a side of the individual needles 1601 and wherein a large side port outlet 1604 is provided according to the seventh example and wherein the tip can be positioned immediately below the distal penetration point of the needle (i.e. the outlet can be positioned closer to the tip while retaining a non-coring configuration when the sloping portion of the tip is non-existent on the side that includes the opening 1604). In the present example the opening 1604 is located within the region of the needle that includes the final taper from shaft width to tip 1616-A wherein at least a portion of the stairsteps 1616-B-1616D associated with the tapering down are located at similar longitudinal heights as portions of the opening and such that the tip position and opening positions configured to place the opening or outlet 1604 under the shielding walls of the tip to provide a non-coring configuration. In some variations of this example, the tip portion of the needle may extend somewhat beyond the outlet or opening to provide shadowing which help further prevent unintentional coring if the entry angle is slightly misaligned.

FIG. 18B provides a perspective view of an alternative configuration for the needles of the seventh example wherein the needle 1601′ includes a central divider 1603 that partitions the outlet 1604-1 and 1604-2 and lumen into two distinct channels having inlets 1605-1 and 1605-2 that extend from the proximal end of the needles to the tip 1616′. In still further variations of the needles of the seventh example, more than two distinct lumens, inlets, and outlets may be provided. In some examples, baffles and mixing elements may be included near the distal end of the needles to provide mixing of multiple drugs just prior to exiting the needle.

Numerous other variations are possible some of which may be derived from features and alternative associated with the other examples of embodiments presented herein before or herein after.

### Eighth Example

### Needle Arrays with Edge Penetration and Multiple Outlets

FIGS. 19A-19B illustrate a further example of a needle designed such that initial tissue contact and penetration occurs at a tip 1716-A that is located at a backside of each individual needle, wherein secondary cutting tips or edges 1716-B, 1716-C and the like exist, and wherein multiple side port outlets 1704-1 and 1704-2 are provided according to an eighth example. A tip side of the needle is visible in FIG. 19A such that an aperture 1704-3 in the tip, flat, or back side may be seen while the multiple stair-stepped edges on the front side can be seen in FIG. 19B.

In variations of this example, the size and position of each of the multiple ports may be selected based on a desired flow distribution. On the back side of the needles an opening (not shown) may exist that is positioned immediately below the needle tip wherein at least a portion of the second tip or edge elements have similar longitudinal heights as does a back window.

As with the other examples and embodiments set forth herein, various alternatives to this example are possible some of which may be based on adding features from one or more other examples or embodiments set forth herein or from their alternatives.

Additional Micro-Needle Device Enhancements

As noted above numerous variations to the presented embodiments are possible, some of which may be based on combinations with one or more features from other embodiments or the variations presented along with such embodiments. Further variations are possible including variations that include features set forth in this portion of the application.

In some alternative embodiments, the micro-needle devices may include multiple apertures with each aperture having a different flow rate, as determined by aperture size, shape, surface texture, and/or the size, shape, and surface texture of the channel leading to the aperture. For example, micro-needle devices with a high flow from distal apertures and a relatively low flow from proximal apertures may be created.

The micro-needle devices in some alternative embodiments may include integrated pressure regulators and/or variable flow restrictors or orifices to regulate flow rate on a local or global basis. Sensing of the backpressure and/or the degree of tissue uptake of a payload may be used to regulate the flow in a closed-loop manner, and delivery profiles (e.g., flow rate as a function of time, flow rate as a function of needle location) may be pre-programmed or dynamically adjusted, with or without closed-loop feedback.

In some embodiments, the micro-needle devices may include built-in filters, including multi-stage filters, which may be monolithically co-fabricated along with the device, and which may be incorporated into the lumen of the micro-needles. Such filters may be used to prevent delivery of particulates within a fluid payload, to prevent clogging of the micro-needle apertures, to prevent aspiration of tissue, etc. Such small diameter pores may also provide wicking or capillary force to help deliver material from the needles to the body of the user.

In some embodiments, the micro-needle devices may include means for vibrating the micro-needles in order to facilitate skin penetration. Such vibration may be along the distal-proximal axis of the needles, or perpendicular to this axis, and may be produced, for example, by piezoelectric elements, fluid pulsation, eccentrically-rotating masses, sound waves, ultrasonic transducers, or the like.

In some embodiments, the micro-needle devices may be externally-actuated or adjusted by the application of a magnetic field, pressure on the skin, sound waves, etc. For example, the devices may include elements such as vanes including ferromagnetic or permanent magnetic materials so that the flow of a fluid through the devices can be varied by application of a suitable magnetic field that may be applied from the outside of the skin or other tissue.

In some embodiments, the apertures in the micro-needles may be variable in size and/or shape, orientation, or configuration such that the apertures are relatively closed during insertion of the micro-needle into the tissue (e.g., to minimize the potential for tissue intrusion/coring, or to prevent premature payload delivery or sampling) and then transition to a relatively open state. The transition may be implemented by a mechanism, by a material that undergoes a phase change (e.g., melting, sublimation) at body temperature or at a brief, artificially-elevated temperature, or by dissolution (e.g., chemical or electrochemical) of a barrier element. In other embodiments, the needles may be self-cleaning. The apertures may become partially obscured or clogged by tissue or other material (e.g., particulates within the payload), but through the motion of an element (e.g., a wiper, plunger, or flap) the material obscuring the apertures is ejected or at least moved to a less-obscuring position. This motion may occur automatically upon insertion or be triggered periodically or as-needed by an external actuator. In some embodiments the tip of the needle may take the form of a plurality of contacting or even overlapping elements that may be pushed apart and even locked open by fluid flow.

In some embodiments, the micro-needle devices may be capable of more than one function, such as delivering a payload and also sampling at the same time or in a time-multiplexed fashion. For example, a device may sample blood glucose levels but also deliver insulin to diabetic patients. The two functions can be combined in a single micro-needle which alternates between a fluid moving proximally-distally for payload delivery, and distally-proximally for sampling. Or, in a two-lumen needle (e.g., with coaxial or side-by-side lumens), two fluid motions can occur simultaneously, each within its dedicated lumen. Alternatively, in an array of needles, some may be dedicated to delivery while others are dedicated to sampling.

In some embodiments, the micro-needle devices may include needles with internal reservoirs, which may obviate the need for an external manifold or reservoir of fluid or other payload material. The reservoirs may take the form of an enlarged portion of the micro-needle lumen, a porous structure, and so forth. Material in fluid form may be retained in the reservoirs by capillary forces, or contained via a cap that can be removed or disrupted. Removal or disruption may be accomplished by a mechanism (e.g., involving piercing by a sharp point), by a material that undergoes a phase change (e.g., melting, sublimation) at body temperature or at a brief, artificially-elevated temperature, by dissolution (e.g., chemical or electrochemical), etc.

The micro-needle devices may in some embodiments include a cap or membrane to prevent a payload in an external reservoir from prematurely entering the needles and potentially leaking out. As before, such a cap may be disrupted by a mechanism, a phase change, dissolution, etc.

In some embodiments, the micro-needle devices comprise mechanisms or sensors to measure the mechanical properties of the surrounding tissue or medium, and use this to signal that penetration of the needle to the desired depth or stratum of tissue has been achieved and no further insertion motion need be applied.

In some embodiments, heating elements may be incorporated into the micro-needle devices so as to induce a healing response, enhance diffusion/uptake or reaction rate of delivered payloads, provide a favorable operating temperature for action of a drug, or to achieve other therapeutic or diagnostic effects. Heating elements may be incorporated into individual needles (e.g., in an array) or in a common base/manifold. In enhanced embodiments and or alternative embodiments, instead of supplying heat, or in addition to supplying heat, the array may be cooled, for example during insertion, to increase the viscosity of the deliverable so that premature delivery does not occur. Such temporary control may be provided by an external heat source or heat absorber or may be supplied by build in heat sources or heat pumps and may be regulated in any desired manner.

In some embodiments, the micro-needle devices may incorporate apertures or lumens through which laser energy can be delivered directly or through inserted optical fibers. Laser energy may be used to create channels in the tissue adjacent to the micro-needle to enhance diffusion and uptake of a payload, facilitate insertion of the needle, etc.

In some embodiments, the micro-needle devices may comprise needles made from materials or incorporating features which enhance visualization (e.g., of needle placement). For example, radiopaque materials may be used for needles overall, or embedded within certain regions (e.g., the tips) of needles. Needles may be textured to enhance ultrasonic imaging and reduce artifacts.

In some embodiments, the micro-needle devices may include an integrated, pre-loaded system for payload delivery (e.g., of a drug) that is substantially fabricated monolithically, e.g., through the EFAB process. Analogous devices may be produced for sampling or other functions.

In some embodiments, micro-needles may be attached to a base or manifold that is flexible so as to allow deformation around curved tissue, or to optimize penetration. Both simple and compound curvature can be achieved through proper design of the base/manifold. Penetration of an array may be optimized by having it shaped to be initially convex (cylindrically or spherically) toward the tissue to be penetrated, thus ensuring that needles toward the center are able to penetrate well, not just those toward the edge of the array. In some embodiments, the base may be substantially rigid, but the needles themselves are able to adjust in length (e.g., through some compliance along the distal-proximal axis, such as a bellows-like sections) and may be provided with individual elements to control the depth of needle penetration into tissue. Thus when the array is pressed against non-planar tissue, each needle penetrates the same amount into the tissue (until stopped by a penetration depth control element such as a widening) by virtue of the compliance.

In some embodiments, micro-needles may be coated internally (and possibly, externally) to provide enhanced compatibility with the payload. Such coatings may be applied on a layer-by-layer basis, or as a post-processing step (e.g., a titanium oxide coating applied using methods commercialized by Chameleon Scientific of Plymouth, Minn.).

In some embodiments, the micro-needle devices may include a ring (e.g., with small needles or spikes to ensure good tissue traction), parallel bars, or other structures which captures, tensions, or compresses tissue outside the region which will be penetrated by the needle array, prior to penetration. Such a ring may be spring-loaded so that the needle array may move toward the skin after the ring has first made contact.

In some embodiments, the micro-needle devices incorporate mixing capability which allows delivery of payloads (e.g., two fluids, a fluid and a suspended solid) which for reasons of limited shelf life or efficacy, befit just form mixing just prior to delivery. For example, static inline mixers may be incorporated into individual needles, with the two materials to be mixed fed to the needles through separate manifolds. The channels of these manifolds may be interleaved with one another. In other embodiments, the mixers may be incorporated into the base or manifold itself. In still other embodiments, the materials may be delivered to the tissue unmixed but mix within the tissue (e.g., through diffusion).

In some embodiments, the micro-needle device may be a single needle or an array of needles that delivers two or more distinct payloads through separate needle lumens and apertures (one or more apertures per lumen). The two materials may be fed to the needle through separate manifolds and the channels of these manifolds may be interleaved with one another. The apertures may be located at different distances along the distal-proximal axis so as to deliver different payloads to different depths. The apertures may be of different sizes and flow rates. The lumens may be coaxial, side-by-side, or otherwise arranged within the needle. The ratio of one material to another may be varied statically or dynamically and controlled independent of environmental factors such as pressure and temperature.

In still further embodiments, the needles that penetrate the tissue may initially be located within other needles and/or hidden behind a membrane. In some embodiments, the needles that penetrate the tissue may be made thinner and thus weaker than would be generally considered allowable. Generally needles are pushed into the a target surface from their proximal ends, with the strength or rigidity of needles selected in combination with the length so that insertion can be completed without undue bending. In some embodiments thin needles are provided with guides that may collapse proximally as the distal end or ends of the needle or needles move into the tissue. Such guides provide added strength needed reliable insertion. In other embodiments needles are feed into using clamps, ratcheting mechanisms (e.g. stepped wings on sides of the needle in combination with fingers that can engage the wings and push the needles distally), or the like that are initially located near the distal ends of the needles and progressively move toward the proximal end as insertion occurs such that the effective length of the needle between insertion point and force application region is reduced.

In some alternative embodiment, skin or other tissue at the insertion point may be distorted or pre-deformed upon insertion and then released to regain its normal configuration.

In some embodiments needles may take on linear or non-linear distally narrowing tapers and needles may have lengths on the millimeter scale or less and be as long as a centimeter or more. The guided needle techniques discussed above may be particularly useful when coupled with such long slender needles.

Various other tip fabrication and sharpening methods are possible and will apparent to those of skill in the art upon review of the teachings herein. As an example of another process, sharp tips are obtained as-fabricated using a plating effect known as mushrooming as with tips used for semiconductor test probes (see U.S. patent application Ser. No. 11/178,145, filed Jul. 7, 2005, which is incorporated herein by reference). In this process, sacrificial metal is electroplated over an island of photoresist to form sloping and/or curved walls, after which the surface is made fully conductive if needed. Then, structural material is plated into the resulting pattern.

The teachings of the various methods of forming tips presented herein and their alternatives may be combined in various combinations to provide new method embodiments for forming tips.

### FURTHER COMMENTS AND CONCLUSIONS

Structural or sacrificial dielectric materials may be incorporated into embodiments of the present invention in a variety of different ways. Such materials may form a third material or higher deposited on selected layers or may form one of the first two materials deposited on some layers. Additional teachings concerning the formation of structures on dielectric substrates and/or the formation of structures that incorporate dielectric materials into the formation process and possibility into the final structures as formed are set forth in a number of patent applications filed Dec. 31, 2003. The first of these filings is U.S. Patent Application No. 60/534,184 which is entitled “Electrochemical Fabrication Methods Incorporating Dielectric Materials and/or Using Dielectric Substrates”. The second of these filings is U.S. Patent Application No. 60/533,932, which is entitled “Electrochemical Fabrication Methods Using Dielectric Substrates”. The third of these filings is U.S. Patent Application No. 60/534,157, which is entitled “Electrochemical Fabrication Methods Incorporating Dielectric Materials”. The fourth of these filings is U.S. Patent Application No. 60/533,891, which is entitled “Methods for Electrochemically Fabricating Structures Incorporating Dielectric Sheets and/or Seed layers That Are Partially Removed Via Planarization”. A fifth such filing is U.S. Patent Application No. 60/533,895, which is entitled “Electrochemical Fabrication Method for Producing Multi-layer Three-Dimensional Structures on a Porous Dielectric”. Additional patent filings that provide teachings concerning incorporation of dielectrics into the EFAB process include U.S. patent application Ser. No. 11/139,262, filed May 26, 2005 by Lockard, et al., and which is entitled “Methods for Electrochemically Fabricating Structures Using Adhered Masks, Incorporating Dielectric Sheets, and/or Seed Layers that are Partially Removed Via Planarization”; and U.S. patent application Ser. No. 11/029,216, filed Jan. 3, 2005 by Cohen, et al., now abandoned, and which is entitled “Electrochemical Fabrication Methods Incorporating Dielectric Materials and/or Using Dielectric Substrates”. These patent filings are each hereby incorporated herein by reference as if set forth in full herein.

Some embodiments may employ diffusion bonding or the like to enhance adhesion between successive layers of material. Various teachings concerning the use of diffusion bonding in electrochemical fabrication processes are set forth in U.S. patent application Ser. No. 10/841,384 which was filed May 7, 2004 by Cohen et al., now abandoned, which is entitled “Method of Electrochemically Fabricating Multilayer Structures Having Improved Interlayer Adhesion” and which is hereby incorporated herein by reference as if set forth in full. This application is hereby incorporated herein by reference as if set forth in full.

Some embodiments may incorporate elements taught in conjunction with other medical devices as set forth in various U.S. patent applications filed by the owner of the present application and/or may benefit from combined use with these other medical devices: Some of these alternative devices have been described in the following previously filed patent applications: (1) U.S. patent application Ser. No. 11/478,934, by Cohen et al., and entitled “Electrochemical Fabrication Processes Incorporating Non-Platable Materials and/or Metals that are Difficult to Plate On”; (2) U.S. patent application Ser. No. 11/582,049, by Cohen, and entitled “Discrete or Continuous Tissue Capture Device and Method for Making”; (3) U.S. patent application Ser. No. 11/625,807, by Cohen, and entitled “Microdevices for Tissue Approximation and Retention, Methods for Using, and Methods for Making”; (4) U.S. patent application Ser. No. 11/696,722, by Cohen, and entitled “Biopsy Devices, Methods for Using, and Methods for Making”; (5) U.S. patent application Ser. No. 11/734,273, by Cohen, and entitled “Thrombectomy Devices and Methods for Making”; (6) U.S. Patent Application No. 60/942,200, by Cohen, and entitled “Micro-Umbrella Devices for Use in Medical Applications and Methods for Making Such Devices”; and (7) U.S. patent application Ser. No. 11/444,999, by Cohen, and entitled “Microtools and Methods for Fabricating Such Tools”. Each of these applications is incorporated herein by reference as if set forth in full herein.

Though the embodiments explicitly set forth herein have considered multi-material layers to be formed one after another. In some embodiments, it is possible to form structures on a layer-by-layer basis but to deviate from a strict planar layer on planar layer build up process in favor of a process that interlaces material between the layers. Such alternative build processes are disclosed in U.S. application Ser. No. 10/434,519, filed on May 7, 2003, now U.S. Pat. No. 7,252,861, entitled Methods of and Apparatus for Electrochemically Fabricating Structures Via Interlaced Layers or Via Selective Etching and Filling of Voids. The techniques disclosed in this referenced application may be combined with the techniques and alternatives set forth explicitly herein to derive additional alternative embodiments. In particular, the structural features are still defined on a planar-layer-by-planar-layer basis but material associated with some layers are formed along with material for other layers such that interlacing of deposited material occurs. Such interlacing may lead to reduced structural distortion during formation or improved interlayer adhesion. This patent application is herein incorporated by reference as if set forth in full.

The patent applications and patents set forth below are hereby incorporated by reference herein as if set forth in full. The teachings in these incorporated applications can be combined with the teachings of the instant application in many ways: For example, enhanced methods of producing structures may be derived from some combinations of teachings, enhanced structures may be obtainable, enhanced apparatus may be derived, and the like.

| US Pat App No., Filing Date | |
|---|---|
| US App Pub No., Pub Date | |
| US Patent No., Pub Date | Inventor, Title |
| 09/493,496 - Jan. 28, 2000 | Cohen, “Method For Electrochemical Fabrication” |
| U.S. Pat. No. 6,790,377 - Sep. 14, 2004 | |
| 10/677,556 - Oct. 1, 2003 | Cohen, “Monolithic Structures Including Alignment and/or |
| 2004-0134772 - Jul. 15, 2004 | Retention Fixtures for Accepting Components” |
| 10/830,262 - Apr. 21, 2004 | Cohen, “Methods of Reducing Interlayer Discontinuities in |
| 2004-0251142A - Dec. 16, 2004 | Electrochemically Fabricated Three-Dimensional Structures” |
| U.S. Pat. No. 7,198,704 - Apr. 3, 2007 | |
| 10/271,574 - Oct. 15, 2002 | Cohen, “Methods of and Apparatus for Making High Aspect |
| 2003-0127336A - Jul. 10, 2003 | Ratio Microelectromechanical Structures” |
| U.S. Pat. No. 7,288,178 - Oct. 30, 2007 | |
| 10/697,597 - Dec. 20, 2002 | Lockard, “EFAB Methods and Apparatus Including Spray |
| 2004-0146650A - Jul. 29, 2004 | Metal or Powder Coating Processes” |
| 10/677,498 - Oct. 1, 2003 | Cohen, “Multi-cell Masks and Methods and Apparatus for |
| 2004-0134788 - Jul. 15, 2004 | Using Such Masks To Form Three-Dimensional Structures” |
| U.S. Pat. No. 7,235,166 - Jun. 26, 2007 | |
| 10/724,513 - Nov. 26, 2003 | Cohen, “Non-Conformable Masks and Methods and |
| 2004-0147124 - Jul. 29, 2004 | Apparatus for Forming Three-Dimensional Structures” |
| U.S. Pat. No. 7,368,044 - May 6, 2008 | |
| 10/607,931 - Jun. 27, 2003 | Brown, “Miniature RF and Microwave Components and |
| 2004-0140862 - Jul. 22, 2004 | Methods for Fabricating Such Components” |
| U.S. Pat. No. 7,239,219 - Jul. 3, 2007 | |
| 10/841,100 - May 7, 2004 | Cohen, “Electrochemical Fabrication Methods Including Use |
| 2005-0032362 - Feb. 10, 2005 | of Surface Treatments to Reduce Overplating and/or |
| U.S. Pat. No. 7,109,118 - Sep. 19, 2006 | Planarization During Formation of Multi-layer Three- |
| | Dimensional Structures” |
| 10/387,958 - Mar. 13, 2003 | Cohen, “Electrochemical Fabrication Method and |
| 2003-022168A - Dec. 4, 2003 | Application for Producing Three-Dimensional Structures |
| | Having Improved Surface Finish“ |
| 10/434,494 - May 7, 2003 | Zhang, “Methods and Apparatus for Monitoring Deposition |
| 2004-0000489A - Jan. 1, 2004 | Quality During Conformable Contact Mask Plating |
| | Operations” |
| 10/434,289 - May 7, 2003 | Zhang, “Conformable Contact Masking Methods and |
| 20040065555A - Apr. 8, 2004 | Apparatus Utilizing In Situ Cathodic Activation of a |
| | Substrate” |
| 10/434,294 - May 7, 2003 | Zhang, “Electrochemical Fabrication Methods With |
| 2004-0065550A - Apr. 8, 2004 | Enhanced Post Deposition Processing” |
| 10/434,295 - May 7, 2003 | Cohen, “Method of and Apparatus for Forming Three- |
| 2004-0004001A - Jan. 8, 2004 | Dimensional Structures Integral With Semiconductor Based |
| | Circuitry” |
| 10/434,315 - May 7, 2003 | Bang, “Methods of and Apparatus for Molding Structures |
| 2003-0234179 A - Dec. 25, 2003 | Using Sacrificial Metal Patterns” |
| U.S. Pat. No. 7,229,542 - Jun. 12, 2007 | |
| 10/434,103 - May 7, 2004 | Cohen, “Electrochemically Fabricated Hermetically Sealed |
| 2004-0020782A - Feb. 5, 2004 | Microstructures and Methods of and Apparatus for |
| U.S. Pat. No. 7,160,429 - Jan. 9, 2007 | Producing Such Structures” |
| 10/841,006 - May 7, 2004 | Thompson, “Electrochemically Fabricated Structures Having |
| 2005-0067292 - May 31, 2005 | Dielectric or Active Bases and Methods of and Apparatus for |
| | Producing Such Structures” |
| 10/434,519 - May 7, 2003 | Smalley, “Methods of and Apparatus for Electrochemically |
| 2004-0007470A - Jan. 15, 2004 | Fabricating Structures Via Interlaced Layers or Via Selective |
| U.S. Pat. No. 7,252,861 - Aug. 7, 2007 | Etching and Filling of Voids” |
| 10/724,515 - Nov. 26, 2003 | Cohen, “Method for Electrochemically Forming Structures |
| 2004-0182716 - Sep. 23, 2004 | Including Non-Parallel Mating of Contact Masks and |
| U.S. Pat. No. 7,291,254 - Nov. 6, 2007 | Substrates” |
| 10/841,347 - May 7, 2004 | Cohen, “Multi-step Release Method for Electrochemically |
| 2005-0072681 - Apr. 7, 2005 | Fabricated Structures” |
| 60/533,947 - Dec. 31, 2003 | Kumar, “Probe Arrays and Method for Making” |
| 60/534,183 - Dec. 31, 2003 | Cohen, “Method and Apparatus for Maintaining Parallelism |
| | of Layers and/or Achieving Desired Thicknesses of Layers |
| | During the Electrochemical Fabrication of Structures” |
| 11/733,195 - Apr. 9, 2007 | Kumar, “Methods of Forming Three-Dimensional Structures |
| 2008-0050524 - Feb. 28, 2008 | Having Reduced Stress and/or Curvature” |
| 11/506,586 - Aug. 8, 2006 | Cohen, “Mesoscale and Microscale Device Fabrication |
| 2007-0039828 - Feb. 22, 2007 | Methods Using Split Structures and Alignment Elements” |
| 10/949,744 - Sep. 24, 2004 | Lockard, “Three-Dimensional Structures Having Feature |
| 2005-0126916 - Jun. 16,2005 | Sizes Smaller Than a Minimum Feature Size and Methods |
| U.S. Pat. No. 7,498,714 - Mar. 3, 2009 | for Fabricating” |

Though various portions of this specification have been provided with headers, it is not intended that the headers be used to limit the application of teachings found in one portion of the specification from applying to other portions of the specification. For example, it should be understood that features of specific embodiments and alternatives acknowledged in association with specific embodiments, are intended to apply to all embodiments to the extent that the features of the different embodiments make such application functional and do not otherwise contradict or remove all benefits of the adopted embodiment. Various other embodiments of the present invention exist. Some of these embodiments may be based on a combination of the teachings herein with various teachings incorporated herein by reference.

In view of the teachings herein, many further embodiments, alternatives in design and uses of the embodiments of the instant invention will be apparent to those of skill in the art. As such, it is not intended that the invention be limited to the particular illustrative embodiments, alternatives, and uses described above but instead that it be solely limited by the claims presented hereafter.

We claim:

1. A device for the intradermal and/or transdermal dispensing of a drug into a body of a patient through a desired delivery area, comprising:
a. a handle
b. a cylindrical body having one or more apertures extending from an interior region to an exterior region wherein the cylindrical body and the handle are joined to one another such that relative rotation between the handle and body may occur,
c. a plurality of needles extending outward from the one or more apertures, wherein the needles and cylindrical body are configured to provide a desired penetration depth into a surface of a delivery area when the cylindrical body is rolled over the delivery area as the handle is translated relative to the delivery area;
wherein the device further includes at least one element taken from the group of elements consisting of:
(1) at least a portion of some of the needles are formed from a multi-layer, multi-material fabrication process where each of the multiple layers are formed from the deposition of at least one structural material and at least one sacrificial material, a trimming (e.g. planarization) of the at least one structural material and the at least one sacrificial material to set a boundary level for the layer, and wherein after formation of a plurality of layers, the sacrificial material is removed from the plurality of layers;
(2) the needles include penetration stops located proximally relative to more distal apertures in the needles such that the distance between the apertures and the penetration stops defines a desired delivery depth for the drug below surface of the delivery area;
(3) the outer surface of the cylindrical body is covered with a membrane that inhibits flow of the drug from the needles except in those locations where the needles have been made to extend through the membrane by contact with the delivery area;
(4) the outer surface of the cylindrical body is covered with a compressible membrane that inhibits flow of the drug from the needles except in those locations where the needles have been made to extend through the membrane by compression of the membrane against the delivery area;
(5) the distal ends of the needles are made to extend from an interior position relative to the apertures in the cylindrical body via one or more bulges in one or more tracks along which with the needles move as the cylindrical body rotates with respect to the handle wherein the bulge or bulges correspond to a location along the perimeter of the cylindrical body that is intended to be in contact with a delivery area during a drug delivery;
(6) a drug is held within the cylindrical body within pores of a flexible porous medium and is forced from selected needles by squeezing the porous medium adjacent the selected needles;
(7) selected needles are extended from selected apertures via fluid pressure exerted by the drug on bases of the selected needles;
(8) a non-rotating inter conformable structure is held with the cylindrical structure wherein the conformable structure includes a recess which acts as a reservoir for the drug and which is configured and located so that it is positioned adjacent to only a portion of the needles and in particular that portion of the needles that are located in a desired dispensing position and wherein other portions of the conformable structure act to inhibit other needles from dispensing the drug;
(9) the distal ends of the needles are made to extend from an interior position relative to the apertures in the cylindrical body such that they extend beyond the outer surface of the cylindrical body for penetration into the tissue through the delivery area when the cylindrical body is in contact with the delivery area;
(10) a plurality of normally closed valves (i.e. when no dispensing is to occur) which can be made to open to allow fluid flow through selected needles when those needles have penetrated the tissue at the delivery area;
(11) a plurality of normally closed valves (i.e. when no dispensing is to occur) which can be made to open to allow fluid flow through selected needles when those needles have penetrated the tissue at the delivery area, wherein selected valves are made to open by a plurality of trigger mechanisms being depressed as the cylindrical body is pressed against the delivery area;
(12) a plurality of normally closed valves (i.e. when no dispensing is to occur) which can be made to open to allow fluid flow through selected needles when those needles have penetrated the tissue at the delivery area, wherein selected valves comprise valve seats that seal the exit ports of the needles when trigger mechanisms are not sufficiently depressed;
(13) a plurality of normally closed valves (i.e. when no dispensing is to occur) which can be made to open to allow fluid flow through selected needles when those needles have penetrated the tissue at the delivery area, wherein selected valves comprise valve seats that seal the fluid entry ports of the needles when trigger mechanisms are not sufficiently depressed;
(14) the plurality of needles are held within the cylindrical body and are made to extend from the cylindrical body in a region of the cylindrical body that is in contact with the delivery area prior to dispensing the drug and after dispensing the drug are made to retract back into the cylindrical body.

2. A device for the intradermal and/or interdermal interaction with a body of a patient, comprising:
a. a handle
b. a smooth body having one or more apertures extending from an interior region to an exterior region wherein the smooth body and the handle are functionally connected to one another such that the smoothed body extends beyond a surface of a body of the handle,
c. a plurality of needles which are extendible from the smooth body and retractable into the smooth body via the one or more apertures such that upon extension a desired intradermal or interdermal interaction can occur via passages within the needles and such that upon retraction the smooth body may be translated or rotated along the surface of the delivery area, wherein the needles and smooth body are configured to provide a desired penetration depth of the needles into a surface of the delivery area when the needles are extended;
wherein the device further includes an element taken from the group of elements consisting of:
(1) at least a portion of some of the needles are formed from a multi-layer, multi-material fabrication process where each of the multiple layers is formed from the deposition of at least one structural material and at least one sacrificial material, a trimming (e.g. planarization) of the at least one structural material and the at least one sacrificial material sets a boundary level for the layer, and wherein after formation of a plurality of layers the sacrificial material is removed from the plurality of layers;
(2) the needles include penetration stops that are located proximally from more distal apertures such that the distance between the apertures and the penetration stops defines a desired interaction depth within the tissue;
(3) the outer surface of the smooth body is covered with a membrane that inhibits interaction between the tissue and the needles except in those locations where the needles have been made to extend through the membrane by contact with the delivery area;
(4) the outer surface of the smooth body is covered with a compressible membrane that inhibits interaction between the needles and the tissue except in those locations where the needles have been made to extend through the membrane by compression of the membrane against the delivery area;
(5) the distal ends of the needles are made to extend from an interior position relative to the apertures in the smooth body via a bulge in one or more tracks along which with the needles move as the smooth body rotates with respect to handle wherein the bulge or bulges correspond to a location along the perimeter of the smooth body that are intended to be in contact with a delivery area an interaction;
(6) a drug is held within the smooth body within pores of a flexible porous medium and is forced from selected needles during an interaction by squeezing the porous medium adjacent the selected needles;
(7) selected needles are extended from selected apertures via fluid pressure exerted by a drug on bases of the selected needles wherein the drug is to be dispensed during an interaction;
(8) a non-rotating conformable structure is held within the smooth structure wherein the conformable structure includes a recess which acts as a reservoir for a drug to be dispensed during an interaction and which is configured and located so that the conformable structure is positioned adjacent to only a portion of the needles and in particular that portion of the needles that are located in a desired dispensing position and wherein other portions of the conformable structure act to inhibit other needles from dispensing the drug;
(9) the distal ends of the needles are made to extend from an interior position relative to the apertures in the smooth body such that they extend beyond the outer surface of the smooth body for penetration into the tissue through the delivery area when the smooth body is in contact with the delivery area;
(10) a plurality of normally closed valves which can be made to open to allow fluid flow through selected needles when those needles have penetrated the tissue at the delivery area, wherein the direction of fluid flow is selected from the group consisting of outward flow and an inward flow;
(11) a plurality of normally closed valves which can be made to open to allow fluid flow through selected needles when those needles have penetrated the tissue at the delivery area, wherein selected valves are made to open by a plurality of trigger mechanisms being depressed as the smooth body is pressed against the delivery area, wherein the direction of fluid flow is selected from the group consisting of outward flow and an inward flow;
(12) a plurality of normally closed valves which can be made to open to allow fluid flow through selected needles when those needles have penetrated the tissue at the delivery area, wherein selected valves comprise valve seats that seal the exit ports of the needles when trigger mechanisms are not sufficiently depressed, wherein the direction of fluid flow is selected from the group consisting of outward flow and an inward flow;
(13) a plurality of normally closed valves which can be made to open to allow fluid flow through selected needles when those needles have penetrated the tissue at the delivery area, wherein selected valves comprise valve seats that seal the fluid entry ports of the needles when trigger mechanisms are not sufficiently depressed, wherein the direction of fluid flow is selected from the group consisting of outward flow and an inward flow;
(14) the plurality of needles are held within the cylindrical body and are made to extend from the cylindrical body in a region of the smooth body that is in contact with the delivery area prior to interaction between the needles and the tissue and after interaction are made to retract back into the smooth body;
(15) the interaction comprises directing radiation down one or more the plurality of needles;
(16) the interaction comprises reading an electrical signal from one or more of the needles;
(17) the interaction comprises applying a voltage to one or more of the needles;
(18) the interaction comprises a diagnostic procedure;
(19) the interaction comprises a preventative treatment; and
(20) the interaction comprises a therapeutic treatment.

3. A method for supplying therapeutic or preventative treatment to a body of a patient, comprising:
(A) applying the surface of a device to the skin of a patient in a desired working area;
(B) extending a plurality of microneedles from the surface of the device through the skin to a desired interaction area;
(C) providing a therapeutic treatment, a preventative treatment to the patent via an interaction wherein the interaction is selected from the group comprising:
(1) delivering antibiotics;
(2) delivering of antipruritic agent;
(3) delivering of anti-inflammatory agent;
(4) delivering of an analgesic;
(5) treating an abscess;
(6) removal of a lesion;
(7) providing a treatment to relieve edema;
(8) delivering depilatory agents to the roots of unwanted hairs;
(9) removing tattoos via dye delivery;
(10) delivering hair re-growth agents;
(11) providing a treatment for hyperhydrosis;
(12) delivery of insulin;
(13) delivery of a vaccine;
(14) delivery of a growth hormone;
(15) delivery of an anti-osteoporosis drug;
(16) delivery a chemotherapy agent;
(17) delivery of a thrombolytic agent;
(18) delivery of a blood pressure regulation drug;
(19) delivery of an addiction therapy drug;
(20) delivery of nanoparticles;
(21) delivery of nanoparticles for use in a cancer treatment;
(22) performing allergy testing;
(23) delivery of radioactive drugs;
(24) delivery of a drug with a narrow therapeutic range; and
(12) providing an in vivo sensing of a body fluid for detection of the presence of a substance of interest.

4. The device of claim 1 wherein the device comprises at least two of the elements.

5. The device of claim 1 wherein the device comprises at least two of the elements.

6. The device of claim 2 wherein the device comprises at least two of the elements.

7. The device of claim 2 wherein the device comprises at least two of the elements.

8. The device of claim 2 wherein the smooth body is configured to roll relative to the handle when contacted to the skin of a patient.

9. The device of claim 8 wherein the interaction occurs via operator control.

10. The device of claim 8 wherein the interaction occurs automatically as rolling occurs.

11. The device of claim 8 wherein the interaction is prompted by signals from the device that can be detected by an operator.

12. The device of claim 2 wherein the smooth body is configured to slide along the skin of the patient.

13. The device of claim 12 wherein the interaction occurs via operator control.

14. The device of claim 12 wherein the interaction occurs automatically as rolling occurs.

15. The device of claim 12 wherein the interaction is prompted by signals from the device that can be detected by an operator.

16. The method of claim 3 wherein the device comprises an element taken from the group of elements consisting of:
(1) at least a portion of some of the needles are formed from a multi-layer, multi-material fabrication process where each of the multiple layers is formed from the deposition of at least one structural material and at least one sacrificial material, a trimming (e.g. planarization) of the at least one structural material and the at least one sacrificial material sets a boundary level for the layer, and wherein after formation of a plurality of layers the sacrificial material is removed from the plurality of layers;
(2) the needles include penetration stops that are located proximally from more distal apertures such that the distance between the apertures and the penetration stops defines a desired interaction depth within the tissue;
(3) the outer surface of the smooth body is covered with a membrane that inhibits interaction between the tissue and the needles except in those locations where the needles have been made to extend through the membrane by contact with the delivery area;
(4) the outer surface of the smooth body is covered with a compressible membrane that inhibits interaction between the needles and the tissue except in those locations where the needles have been made to extend through the membrane by compression of the membrane against the delivery area;
(5) the distal ends of the needles are made to extend from an interior position relative to the apertures in the smooth body via a bulge in one or more tracks along which with the needles move as the smooth body rotates with respect to handle wherein the bulge or bulges correspond to a location along the perimeter of the smooth body that are intended to be in contact with a delivery area an interaction;
(6) a drug is held within the smooth body within pores of a flexible porous medium and is forced from selected needles during an interaction by squeezing the porous medium adjacent the selected needles;
(7) selected needles are extended from selected apertures via fluid pressure exerted by a drug on bases of the selected needles wherein the drug is to be dispensed during an interaction;
(8) a non-rotating conformable structure is held within the smooth structure wherein the conformable structure includes a recess which acts as a reservoir for a drug to be dispensed during an interaction and which is configured and located so that the conformable structure is positioned adjacent to only a portion of the needles and in particular that portion of the needles that are located in a desired dispensing position and wherein other portions of the conformable structure act to inhibit other needles from dispensing the drug;
(9) the distal ends of the needles are made to extend from an interior position relative to the apertures in the smooth body such that they extend beyond the outer surface of the smooth body for penetration into the tissue through the delivery area when the smooth body is in contact with the delivery area;
(10) a plurality of normally closed valves which can be made to open to allow fluid flow through selected needles when those needles have penetrated the tissue at the delivery area, wherein the direction of fluid flow is selected from the group consisting of outward flow and an inward flow;
(11) a plurality of normally closed valves which can be made to open to allow fluid flow through selected needles when those needles have penetrated the tissue at the delivery area, wherein selected valves are made to open by a plurality of trigger mechanisms being depressed as the smooth body is pressed against the delivery area, wherein the direction of fluid flow is selected from the group consisting of outward flow and an inward flow;
(12) a plurality of normally closed valves which can be made to open to allow fluid flow through selected needles when those needles have penetrated the tissue at the delivery area, wherein selected valves comprise valve seats that seal the exit ports of the needles when trigger mechanisms are not sufficiently depressed, wherein the direction of fluid flow is selected from the group consisting of outward flow and an inward flow;
(13) a plurality of normally closed valves which can be made to open to allow fluid flow through selected needles when those needles have penetrated the tissue at the delivery area, wherein selected valves comprise valve seats that seal the fluid entry ports of the needles when trigger mechanisms are not sufficiently depressed, wherein the direction of fluid flow is selected from the group consisting of outward flow and an inward flow;
(14) the plurality of needles are held within the cylindrical body and are made to extend from the cylindrical body in a region of the smooth body that is in contact with the delivery area prior to interaction between the needles and the tissue and after interaction are made to retract back into the smooth body;
(15) the interaction comprises directing radiation down one or more the plurality of needles;
(16) the interaction comprises reading an electrical signal from one or more of the needles;
(17) the interaction comprises applying a voltage to one or more of the needles;
(18) the interaction comprises a diagnostic procedure;
(19) the interaction comprises a preventative treatment; and
(20) the interaction comprises a therapeutic treatment.

17. The method of claim 16 wherein the device comprises at least two of the elements.

18. The method of claim 3 wherein the smooth body is configured to roll relative to the handle when contacted to the skin of a patient.

19. The device of claim 18 wherein the interaction occurs via operator control.

20. The device of claim 18 wherein the interaction occurs automatically as rolling occurs.

21. The device of claim 18 wherein the interaction is prompted by signals from the device that can be detected by an operator.

22. The device of claim 3 wherein the smooth body is configured to slide along the skin of the patient.

23. The device of claim 22 wherein the interaction occurs via operator control.

24. The device of claim 22 wherein the interaction occurs automatically as rolling occurs.

25. The device of claim 22 wherein the interaction is prompted by signals from the device that can be detected by an operator.
